(19) United States
(12) Patent Application Publication
(10) Pub. No.: US 2023/0265081 A1
(43) Pub. Date: Aug. 24, 2023

(54) METHODS OF TREATING CORONAVIRUS

(71) Applicant: VERU INC., Miami, FL (US)

(72) Inventors: Mitchell S. Steiner, Germantown, TN (US); Kester Gary Barnette, Wake Forest, NC (US)

(73) Assignee: VERU INC., Miami, FL (US)

(21) Appl. No.: 18/133,497

(22) Filed: Apr. 11, 2023

Related U.S. Application Data
(63) Continuation-in-part of application No. 17/222,835, filed on Apr. 5, 2021, now Pat. No. 11,648,236.
(60) Provisional application No. 63/145,886, filed on Feb. 4, 2021.
(60) Provisional application No. 63/004,781, filed on Apr. 3, 2020.
(60) Provisional application No. 63/340,122, filed on May 10, 2022.
(60) Provisional application No. 63/329,601, filed on Apr. 11, 2022.
(60) Provisional application No. 63/145,886, filed on Feb. 4, 2021.

(51) Int. Cl. C07D 403/04; A61K 45/06; A61P 31/14

(52) U.S. Cl. CPC C07D 403/04; A61K 45/06; A61P 31/14

(57) ABSTRACT

The present invention relates to methods of treating coronavirus infections using compounds having anti-tubulin or tubulin disruption activity.

### CROSS REFERENCE TO RELATED APPLICATIONS

This application claims the benefit of priority to U.S. Provisional Application Nos. 63/145,886, filed Feb. 4, 2021; 63/329,601, filed on Apr. 11, 2022; and 63/340,122, filed on May 10, 2022 and this application is a continuation-in-part of U.S. application Ser. No. 17/222,835, filed on Apr. 5, 2021 which claims the benefit of priority to U.S. Provisional Application Nos. 63/004,781, filed Apr. 3, 2020; and 63/145,886, filed Feb. 4, 2021, hereby incorporated by reference.

### FIELD OF THE INVENTION

The present invention is directed to methods of treating a coronavirus, using compounds having cytoskeleton disruptor activity, and formulations including the compounds with pharmaceutical acceptable excipients and/or additional cytoskeleton disruptor compounds.

### BACKGROUND OF THE INVENTION

Over the last 20 years, a number of viral epidemics have posed a serious global public health risk including Severe Acute Respiratory Syndrome coronavirus (SARS-CoV) in 2002-2003, the Middle East Respiratory Syndrome coronavirus (MERS-CoV) in 2012, and Ebola in 2014-2016. On Nov. 17, 2019, a new viral acute severe respiratory disease emerged in Wuhan, China. In February 2020, World Health Organization (WHO) announced the disease's name as COVID-19 for COronaVIrus Disease first discovered in the year 2019. The coronavirus causing the disease, as it was similar to SARS-CoV, was eventually named by the International Committee on Taxonomy of Viruses (ICTV) as SARS-CoV-2. COVID-19 (SARS-CoV-2) has been declared a pandemic with over 804,061 cases and 39,074 deaths worldwide and counting as of Mar. 31, 2020. By March 2021, these numbers increased to 128,109,427 cases and 2,800,279 deaths (3%) worldwide with 103,307,591 confirmed recoveries (97%). Vaccines for SARS-CoV-2 began to be approved in the United States in December 2020 with three emergency use authorizations (EUAs) provided by March 2021; however, herd immunity has not yet reached. Despite ongoing worldwide social distancing and immunization efforts, 518,201 active cases remain including about 100,000 critically ill patients currently worldwide.

Coronaviruses are enveloped positive-sense single-stranded RNA viruses. They infect birds and mammals, especially their respiratory and gastrointestinal systems. Due to high mutation and recombination rates in coronaviruses, frequent host-shifting events from animal-to-animal and animal-to-human have occurred. Bats were identified as a natural reservoir during the severe acute respiratory syndrome (SARS) outbreak.

SARS-CoV-2, is an enveloped, nonsegmented, positive-sense, single stranded RNA virus with an unusually large RNA genome, a nucleocapsid, and club-like spikes that project from their surface called spike (S) protein. It belongs to the betacoronavirus category which includes SARS-CoV and MERS-CoV. These viruses have been responsible for epidemics with variable severity with both respiratory and extra-respiratory clinical manifestations, highly contagious, and mortality rates between 10-35%. The Coronavirus superfamily (Coronaviridae) includes several human pathogens with large RNA genomes, e.g., viral encephalitis, and they are classified into alpha, beta, delta, and gamma coronavirus families and then further divided into Lineages A, B, C, and D. SARS-CoV-2 is a Lineage B betacoronavirus.

The clinical spectrum of SARS-CoV-2 varies from asymptomatic to clinical conditions characterized by pneumonia with respiratory failure necessitating mechanical ventilation and support in an intensive care unit (ICU) to sepsis, septic shock, and multiple organ failure. Chinese CDC clinical presentation reported the following disease classifications and rates of mild, severe, and critical disease in the Chinese population infected with SARS-CoV-2 in 2019-2020 which appear to be similar in other infected populations: (1) Mild disease (81%): symptoms of an upper respiratory tract viral infection, including mild fever, cough (dry), sore throat, nasal congestion, headache, muscle pain, or malaise. Signs of a more serious disease, such as dyspnea, are not present; (2) Severe disease (14%): dyspnea, respiratory frequency ≥30 breaths/min, blood oxygen saturation (SpO2)≤93%, PaO2/FiO2 ratio or P/F [the ratio between the blood pressure of the oxygen (partial pressure of oxygen, PaO2) and the percentage of oxygen supplied (fraction of inspired oxygen, FiO2)]<300, and/or lung infiltrates >50% on imaging study within 24 to 48 hours; and (3) Critical disease (5%): respiratory failure, septic shock, and/or multiple organ dysfunction. In some cases, an abnormal immune system over reaction takes place which has been labeled a ‘cytokine storm. The cytokine storm is clinically manifested as an acute systemic inflammatory syndrome characterized by fever and multiple organ dysfunction. Cytokines and chemokines are induced by the viral infection which over-activates an inflammatory response (e.g., NLRP3 inflammasomes activation) which can lead to septic shock and extensive tissue damage.

The spectrum of disease and pharmacotherapy of COVID-19 as of March 2021 (unless otherwise specified) is summarized in the following paragraphs: Potential therapeutic drug classes for COVID-19 include antibody, antiviral, and anti-inflammatory therapies. Early in the time course of infection, the severity of disease is relatively minor and treatment can be focused on prevention of virus entering cells (antibody therapies) or inhibition of virus replication (antiviral therapies). Antiviral drugs, Paxlovid (viral protease inhibitor) and molnupiravir (nucleoside analogue), were developed by pharmaceutical companies Pfizer and Merck to prevent people who are at high risk from becoming severely ill after infection with the SARS-CoV-2 virus. The Food and Drug Administration (FDA) issued emergency use authorizations (EUA's) in late 2021 for both medications. In more severe cases, the patient progresses to include pulmonary infection, in which case, the addition of anti-inflammatory therapy is recommended. For example, at the time of writing, hospitalized patients typically get remdesivir (antiviral) and dexamethasone (anti-inflammatory) as standard of care, whereas mild to moderate non-hospitalized with high risk for progression to critical disease may receive an antiviral therapy alone. When pulmonary infection is present, it can progress to severe acute respiratory syndrome (SARS) in which case it is necessary to supplement oxygen including by mechanical ventilation or extracorporeal membrane oxygenation (ECMO). In this later SARS phase of COVID-19 infection, an overwhelming inflammatory response is the primary cause of damage to the respiratory system leading to acute respiratory distress syndrome (ARDS), necessitating the use of anti-inflammatory therapies which have limited efficacy data, less evidence for the efficacy of antivirals, and no promising efficacy data for antibodies in SARS.

Despite multiple EUA's and an approval, pharmacotherapeutic treatment efficacies of COVID-19 early infection and SARS are modest and drug treatment at all points in the course of disease remains an unmet clinical need. Unfortunately, the principal treatment for SARS remains supportive care and oxygen therapy for patients with severe infection. Mechanical ventilation or ECMO may be necessary in cases of respiratory failure refractory to oxygen therapy, whereas hemodynamic support is essential for managing septic shock. The overall mortality rate for individuals with a SARS-CoV-2 infection appears to be 3% to 4% and as high as 40% for patients with WHO severity scores of >4. Accordingly, current pharmacotherapeutic treatments available as of March 2021 are discussed as potential therapeutic classes. For example, only remdesivir is approved as an antiviral and has very limited efficacy, whereas dexamethasone is recommended as an EUA anti-inflammatory treatment. Further, there is a rapidly evolving series of other novel and repurposed therapies used under emergency use authorization (EUA) which is briefly summarized below. Moreover, many drugs such as hydroxychloroquine gained widespread use based on indirect evidence or case studies that were later refuted by randomized clinical trials. Others in this category include vitamins C and D, zinc, famotidine, ivermectin, ACEI/ARBs, and antibacterials such as azithromycin.

Antibody therapies such as convalescent plasma, IVIG (Intravenous IgG) (not discussed below; see PMID: 33087047 for more information), and neutralizing antibodies (casirivimab plus imdevimab; bamlanivimab; bamlanivimab plus etesevimab; etc.) are considered most likely to be effective early in the time course of infection as these are intended to prevent cell entry by binding to and neutralizing viral spike (S) proteins, thereby blocking the binding to cell receptors and co-receptors and preventing viral entry into cells. None of the antibody therapies are FDA approved, however, several were given EUA including convalescent plasma in August 2020, both casirivimab plus imdevimab (received EUA if administered together) and bamlanivimab monotherapy in November 2020, whereas bamlanivimab plus etesevimab received EUA in February 2021. Administered early in the course of disease, FDA indicated that transfusion of high titer COVID-19 convalescent plasma had the potential for clinical benefit. Alternatively, casirivimab plus imdevimab (REGEN-COV™; two recombinant human monoclonal antibodies that bind to nonoverlapping epitopes of the spike (S) protein receptor-binding domain (RBD) of the SARS-CoV-2 virus) received EUA for the treatment of mild to moderate COVID-19 in adults, as well as in pediatric patients at least 12 years of age and weighing at least 40 kg, who have received positive results of direct SARS-CoV-2 viral testing and are at high risk for progressing to severe COVID-19 and/or hospitalization. On Mar. 23, 2021, Regeneron released Phase 3 data for a treated population of infected non-hospitalized patients (n=4,567) suggesting that this combination reduced hospitalization or death by 70% in non-hospitalized COVID-19 patients; further supporting its use in an outpatient setting (https://investor.regeneron.com/news-releases/news-release-details/phase-3-trial-shows-regen-covtm-casirivimab-imdevimab-antibody). Bamlanivimab monotherapy (a recombinant neutralising human IgG1κ monoclonal antibody that also binds to the RBD of the S protein of SARS-CoV-2 and prevents the attachment of S protein with the human ACE2 (a cell surface protein) receptor) received EUA for the same indication as REGEN-COV. EUA was also granted for the combination of bamlanivimab plus etesevimab (these bind to different but overlapping epitopes in the RBD of the S protein; using both antibodies together is expected to reduce the risk of viral resistance) for the same indication as the other synthetic neutralizing antibodies. The benefit of treatment with monoclonal neutralizing antibodies has not been observed in patients hospitalized due to COVID-19 and may be associated with worse clinical outcomes when administered to hospitalized patients requiring high flow oxygen or mechanical ventilation with COVID-19. In overview, none of the antibody therapies are FDA approved as of May 2022 but rather some of them still have active EUA's for use in early infection in patients at high risk for progression. Other antibody pharmacotherapies not mentioned above have also received EUA by May 2022 and are discussed elsewhere herein.

Certain hospitalized adult and pediatric COVID-19 patient populations are candidates for the only FDA approved therapy, an antiviral remdesivir (approved as Veklury). Remdesivir is a nucleotide prodrug for intravenous use that inhibits RNA polymerase of SARS-CoV-2. On Oct. 22, 2020, FDA approved Veklury (remdesivir) for use in adults and pediatric patients (12 years of age and older and weighing at least 40 kg) for the treatment of COVID-19 requiring hospitalization. Veklury should only be administered in a hospital or in a healthcare setting capable of providing acute care comparable to inpatient hospital care. This approval does not include the entire population that had been authorized to use Veklury under an EUA issued on May 1, 2020. Access for pediatric populations via the EUA continues for emergency use by licensed healthcare providers. The EUA allows treatment of suspected or laboratory-confirmed COVID-19 in hospitalized pediatric patients weighing 3.5 kg to less than 40 kg or hospitalized pediatric patients less than 12 years of age weighing at least 3.5 kg. Treatment algorithms are still uncertain for COVID-19 patients but some studies suggest modest mortality benefit of remdesivir in hypoxia patients on supplemental oxygen (ACTT-1 study) and severely ill patients not on mechanical ventilation (SIMPLE study), however, use of remdesivir in mechanically ventilated patients was not associated with a significant reduction of mortality (PMID: 33204761). Accordingly, as of January 2021, for hospitalized patients who require mechanical ventilation or ECMO, NIH recommends dexamethasone monotherapy, not Veklury mono- or combination therapy.

Critically ill patients with COVID-19 may be best served via use of dexamethasone (equivalent alternatives to dexamethasone, i.e., corticosteroids, are acceptable) since most of the damage is from immune overreaction in the lung. Though dexamethasone use via EUA continues (March 2021), the RECOVERY randomized clinical trial only demonstrated modest improvements in 28-day mortality with dexamethasone in all hospitalized patents (22.9% for dexamethasone vs. 25.7% for usual care), but improved outcomes for higher oxygenation requirement subgroups (PMID: 32678530). Similarly, treatment recommendations are stratified by oxygenation requirement with dexamethasone monotherapy is strongly recommended by NIH for those hospitalized on invasive mechanical ventilation or ECMO. Recommendations change to dexamethasone monotherapy or the addition of remdesivir for those hospitalized on non-invasive ventilation, whereas those hospitalized on supplemental oxygen can receive remdesivir or dexamethasone monotherapy, or their combination. However, dexamethasone is not recommended for those patients that are not hospitalized or hospitalized without supplemental oxygen requirement. Thus far, all recommendations are based on limited evidence and World Health Organization (WHO) recommendations differ significantly from NIH. For example, per WHO, remdesivir is not recommended regardless of severity of illness; however, WHO agrees with systemic corticosteroids for severe and critical COVID-19.

Other unapproved anti-inflammatory therapies include IL-6 inhibitors (tocilizumab), interferons, IL-1 inhibitors, and kinase inhibitors, however, as of February 2021, NIH (www.covid19treatmentguidelines.nih.gov) either indicates insufficient data or recommends against the routine use of these agents. One exception is baricitinib, a JAK inhibitor approved for rheumatoid arthritis, which as of November 2020 has EUA in combination with remdesivir for hospitalized patients with mild, moderate and severe COVID-19. EUA states for the combination is for emergency use by healthcare providers for the treatment of suspected or laboratory-confirmed COVID-19 in hospitalized adults and pediatric patients 2 years of age or older requiring supplemental oxygen, invasive mechanical ventilation, or extracorporeal membrane oxygenation (ECMO).

As can be seen, SARS-CoV-2 pharmacotherapy is based on limited data and current agents have limited efficacy at preventing early infection from progressing and decreasing mortality in SARS. Correspondingly, better SARS-CoV-2 pharmacotherapy is urgently needed not just for the current global pandemic but also for future viral epidemics and pandemics, or in the case the SARS-CoV infections become endemic. The instant invention is intended to treat SARS-CoV-2 as well as future epidemics and pandemics derived from the Coronaviridae family which typically produce hyperinflammatory lung infections, and despite emerging and existing therapies carry a high morbidity and mortality burden. Viruses have efficient mechanisms that take control of their host's cellular machinery to carry out viral replication, assembly, and to exit (egress) from the cell to spread infectious virions. Given the spatial distances between the point of virion entry at the plasma membrane to the location in the cell where RNA replication (nucleus) and viral assembly occur in the endoplasmic reticulum and Golgi, and then the newly generated virions have to travel back out to the plasma membrane to egress out of the cell, it is no surprise that the virus's most critical initial task is to hijack the host's internal transportation system, the cytoskeleton. The cytoskeleton is composed of three major types of protein filaments: microfilaments (actin), microtubules (tubulin), and intermediate filaments. The principal ones involved in viral replication and trafficking (transport) are microtubules and microfilaments since these are two main filament systems involved in intracellular transport.

Microtubules are important for cell shape, transport, motility, and cell division. Microtubules are dynamic long polar fibers/filaments that result from the polymerization of α and β tubulin heterodimer subunits with a positive end located at the plasma membrane and a minus end facing the nucleus at the microtubule organizing center (MTOC). From the MTOC, microtubule fibers radiate out from the nuclear area towards the periphery of the cell. Microtubules are dynamic network systems, meaning that, they undergo rapid polymerization adding α and β tubulin subunits heterodimers together to create a growing polymer chain, and subsequent rapid depolymerization (remove α and β tubulin subunits heterodimers) to deconstruct and shrink the polymer chain. This “dynamic” growing and shrinking ability of microtubules serves the constantly changing transportation requirements of the cell. Large macromolecules, like viruses, engage with specialized motor proteins (kinesins and dyneins). Kinesins and dyneins attach, carry, and move the virus cargo up and down these microtubule tracks, like train cars, to travel long distance to reach the different compartments within the cell.

As many human and animal coronaviruses originated from bats and most eukaryotic cells contain microtubules, there appears to be a conserved microtubule dependent coronavirus replication across species. Furthermore, viruses may have evolved microtubule-binding motifs or similar amino acid sequences complementary to motifs in kinesins and dyneins for successful trafficking interactions. Coronaviruses like Mouse Hepatitis Virus CoV use microtubules for neuronal spread and the Feline Infectious Peritonitis Virus (FIPV) is transported by microtubules toward the MTOC. For the porcine transmissible gastroenteritis virus (TGEV), upregulation of both α and β tubulin subunits occurs after infection. Thus, focusing on the cytoskeleton network as a drug target with the goal of impairing intracellular trafficking and disrupting virus and host interactions may be an effective way to treat coronavirus infections.

Viruses are obligate intracellular parasites, and therefore, depend solely on the cellular machinery for membrane trafficking, nuclear import and export, and gene expression. Incoming viral particles move from the cell surface to intracellular sites of viral transcription and replication. During assembly and egress, subviral nucleoprotein complexes and virions travel back to egress the plasma membrane. Because diffusion of large molecules is severely restricted in the cytoplasm, viruses use ATP-hydrolyzing molecular motors of the host for propelling along the microtubules, which are the intracellular highways.

Microtubules are cytoskeletal filaments consisting of α- and β-tubulin heterodimers and are involved in a wide range of cellular functions, including shape maintenance, vesicle transport, cell motility, and division. Tubulin is the major structural component of the microtubules and a verified target for a variety of antiviral drugs. Compounds that are able to interfere with microtubule-tubulin equilibrium in cells are effective in the treatment of viruses as a virus generally uses microtubules as a source of transportation within the cell. Other compounds that interfere with microtubule-tubulin equilibrium in cells, such as paclitaxel and vinblastine, are limited by their toxicity.

Drugs that target the cytoskeleton, especially the microtubule components, are important therapeutic agents for cancer and inflammation. The clinical activity of these compounds is dictated by the location that these compounds bind on the α and β-tubulin heterodimers that compose the microtubule filament. Three major binding sites on α and β-tubulin subunits have been identified as taxanes-, vinca alkaloid-, and colchicine-binding sites. Such drugs are commonly classified into two major categories: microtubule-stabilizing (e.g., taxanes) and microtubule-destabilizing, or depolymerizing agents (e.g., vinca alkaloids and colchicine).

Colchicine has a narrow therapeutic index with no clear distinction between nontoxic, toxic, and lethal doses. Metabolically, colchicine is eliminated via P-glycoprotein (P-gp; also known as Multi-Drug Resistance 1 (MDR1) protein). Drug-drug interactions are common with CYP3A4 and P-glycoprotein inhibitors which can increase colchicine blood concentrations to toxic levels leading to colchicine poisoning and death. Life-threatening and fatal toxicities have been observed when colchicine is administered with P-gp or strong CYP3A4 inhibitors even at approved therapeutic doses. Additional serious toxicities including myelosuppression, disseminated intravascular coagulation, and cell damage in renal, hepatic, circulatory, and central nervous systems have been observed with approved therapeutic doses of colchicine. These observed serious adverse events limit the clinical use of colchicine.

The antiviral activity of combretastatin, colchicine, and colchicine derivatives and their selected prodrugs against DENV and ZIKV in cell culture was observed at low micromolar and sub-micromolar concentrations. A major problem with taxanes, as with many biologically active natural products, is its lipophilicity and lack of solubility in aqueous systems. This leads to the use of emulsifiers like Cremophor EL and Tween 80 in clinical preparations, which leads to serious hypersensitivity reactions.

Nocodazole is a synthetic compound identified in a screen for anthelminthic agents. Nocodazole is a microtubule depolymerization agent as it binds to free tubulin heterodimers and prevents them from incorporating into microtubules. It has not been used clinically because of poor bioavailability and high toxicity.

The cellular and viral solution to master intracellular trafficking is an organized network or filaments including microtubules. Cells require microtubules for long-term normal physiology, and viruses are obligate intracellular parasites that completely depend on the physiology of the host cell. Thus, it is no surprise that most, if not all, viral life cycles require microtubules for efficient replication. The viral binding sites on microtubules might provide new targets for antiviral therapy. The inventions of this application address a novel method of interfering with microtubules of the cytoskeleton to prevent virus intracellular transportation, replication, and egress.

### SUMMARY OF THE INVENTION

The invention encompasses methods of treating a coronavirus infection in a subject in need thereof by administering to the subject a formulation having a therapeutically effective amount of a compound of Formula (I):

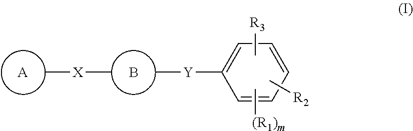

wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

In an embodiment of the invention, the method encompasses compounds of Formula I wherein
A is phenyl or indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

In another embodiment of the invention, the method encompasses compounds of Formula I wherein A is phenyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

In yet another embodiment of the invention, the method encompasses compounds of Formula I wherein
A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

An embodiment of the invention, the method encompasses compounds of Formula I wherein A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond;
Y is —C═O; and
m is 1-3, or a pharmaceutically acceptable salt, hydrate, polymorph, or isomer thereof.

Another embodiment of the invention encompasses methods of treating a coronavirus infection in a subject in need thereof by administering to the subject a formulation having a therapeutically effective amount of a compound of the Formula VII:

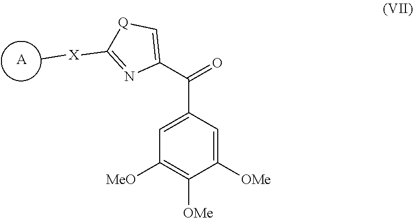

wherein
X is a bond or NH;
Q is S or NH; and
A is a phenyl, indolyl, or indazolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof. In another embodiment of the invention, the method encompasses compounds of Formula VII wherein X is S. In another embodiment of the invention, the method encompasses compounds of Formula VII wherein X is NH. In yet another embodiment of the invention, the method encompasses compounds of Formula VII, wherein X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

An embodiment of the invention encompasses methods of treating a coronavirus infection in a subject in need thereof by administering to the subject a formulation having a therapeutically effective amount of a compound of the Formula VII(c):

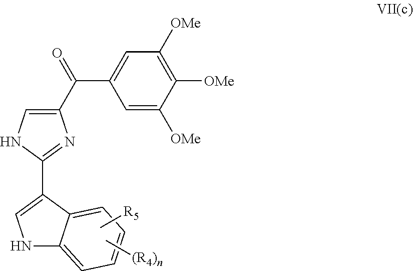

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Another embodiment of the invention, encompasses methods of treating a coronavirus infection in a subject in need thereof by administering to the subject a formulation having a therapeutically effective amount of a compound 17ya represented:

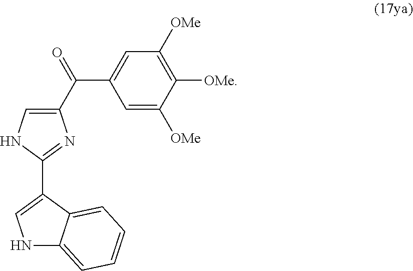 [SMILES: COc1cc(C(=O)c2c[nH]c(-c3c[nH]c4ccccc34)n2)cc(OC)c1OC]

Yet another embodiment of the invention encompasses methods of treating viral infections wherein the viral infection is caused by a Coronaviridae virus. An embodiment of the invention encompasses methods of treating coronavirus infections wherein the infection is caused by SARS-CoV, MERS-CoV, COVID-19 or SARS-CoV-2. Another embodiment of the invention encompasses methods of treating coronavirus infections wherein the infection is caused by COVID-19.

An embodiment of the invention encompasses methods of treating viral infections in which the infection is caused by a coronavirus. Another embodiment of the invention encompasses, methods of treating a coronavirus infection caused by SARS-CoV, MERS-CoV, or SARS-CoV-2. A preferred embodiment of the invention encompasses methods of treating a subject with SARS-CoV-2 infection. A further embodiment of the invention encompasses methods of treating a subject with SARS-CoV-2 infection at high risk for acute respiratory distress syndrome (ARDS) or severe acute respiratory syndrome (SARS). Another embodiment of the invention encompasses methods wherein treating a subject with SARS-CoV-2 infection reduces respiratory failure and/or mortality. Another embodiment of the invention encompasses methods wherein treating a subject with SARS-CoV-2 infection reduces viral load. Another embodiment of the invention encompasses methods wherein treating a subject with SARS-CoV-2 infection at high risk for acute respiratory distress syndrome (ARDS) or severe acute respiratory syndrome (SARS) reduces mortality. Another embodiment of the invention encompasses methods wherein treating a subject with SARS-CoV-2 infection reduces morbidity. Another embodiment of the invention encompasses methods wherein treating a subject with SARS-CoV-2 infection reduces morbidities including atrial fibrillation, bradycardia, pneumonia, bacterial pneumonia, hyperkalemia, hypokalemia, hypophosphatemia, chronic bronchitis, hypoxia, pneumothorax, respiratory failure, acute renal injury, cardiac arrest, septic shock, or hypotension, or any combination thereof. Another embodiment of the invention encompasses methods wherein treating a subject with SARS-CoV-2 infection reduces morbidities including respiratory failure, acute renal injury, cardiac arrest, septic shock, or hypotension, or any combination thereof. Another embodiment of the invention encompasses methods wherein treating a subject with SARS-CoV-2 infection at high risk for acute respiratory distress syndrome (ARDS) or severe acute respiratory syndrome (SARS) reduces morbidity. Another embodiment of the invention encompasses methods wherein treating a subject with SARS-CoV-2 infection reduces respiratory failure, days in ICU, days on mechanical ventilator, or improves WHO Ordinal Scale for Clinical Improvements. Another embodiment of the invention encompasses methods wherein treating a subject with SARS-CoV-2 infection reduces days in the mechanical ventilation. Another embodiment of the invention encompasses methods wherein treating a subject with SARS-CoV-2 infection reduces days in the ICU. Another embodiment of the invention encompasses methods wherein treating a subject with SARS-CoV-2 infection reduces days in the hospital. Another embodiment of the invention encompasses methods wherein treating a subject with SARS-CoV-2 infection reduces mortality. Another embodiment of the invention encompasses methods wherein treating a subject with SARS-CoV-2 infection improves WHO Ordinal Scale for Clinical Improvements. Another embodiment of the invention encompasses methods wherein treating a subject with SARS-CoV-2 infection reduces morbidity. Another embodiment of the invention encompasses methods wherein treating a subject with SARS-CoV-2 infection reduces days on the mechanical ventilator, days in the ICU, days in the hospital, mortality, morbidity, or improves WHO Ordinal Scale for Clinical Improvements, or any combination thereof.

Another embodiment of the invention encompasses methods wherein treating a subject with SARS-CoV-2 infection at high risk for acute respiratory distress syndrome (ARDS) or severe acute respiratory syndrome (SARS) reduces days in the mechanical ventilation. Another embodiment of the invention encompasses methods wherein treating a subject with SARS-CoV-2 infection at high risk for acute respiratory distress syndrome (ARDS) or severe acute respiratory syndrome (SARS) reduces days in the ICU. Another embodiment of the invention encompasses methods wherein treating a subject with SARS-CoV-2 infection at high risk for acute respiratory distress syndrome (ARDS) or severe acute respiratory syndrome (SARS) reduces days in the hospital. Another embodiment of the invention encompasses methods wherein treating a subject with SARS-CoV-2 infection at high risk for acute respiratory distress syndrome (ARDS) or severe acute respiratory syndrome (SARS) reduces mortality. Another embodiment of the invention encompasses methods wherein treating a subject with SARS-CoV-2 infection at high risk for acute respiratory distress syndrome (ARDS) or severe acute respiratory syndrome (SARS) improves WHO Ordinal Scale for Clinical Improvements. Another embodiment of the invention encompasses methods wherein treating a subject with SARS-CoV-2 infection at high risk for acute respiratory distress syndrome (ARDS) or severe acute respiratory syndrome (SARS) reduces morbidity. Another embodiment of the invention encompasses methods wherein treating a subject with SARS-CoV-2 infection at high risk for acute respiratory distress syndrome (ARDS) or severe acute respiratory syndrome (SARS) reduces days on the mechanical ventilator, days in the ICU, days in the hospital, mortality, morbidity, or improves WHO Ordinal Scale for Clinical Improvements, or any combination thereof.

Another embodiment of the invention encompasses methods wherein treating a subject with SARS-CoV-2 infection at high risk for acute respiratory distress syndrome (ARDS) or severe acute respiratory syndrome (SARS) reduces respiratory failure, days in ICU, days on mechanical ventilator, or improves WHO Ordinal Scale for Clinical Improvements. Another embodiment of the invention encompasses methods wherein treating a subject with SARS-CoV-2 infection reduces mortality or respiratory failure in subjects >60 years of age. Another embodiment of the invention encompasses methods wherein treating a subject with SARS-CoV-2 infection at high risk for acute respiratory distress syndrome (ARDS) or severe acute respiratory syndrome (SARS) reduces mortality or respiratory failure in subjects >60 years of age. Another embodiment of the invention encompasses methods wherein treating a subject with SARS-CoV-2 infection reduces mortality or respiratory failure when dosed in combination with remdesivir and/or dexamethasone. Another embodiment of the invention encompasses methods wherein treating a subject with SARS-CoV-2 infection at high risk for acute respiratory distress syndrome (ARDS) or severe acute respiratory syndrome (SARS) reduces mortality or respiratory failure when dosed in combination with remdesivir and/or dexamethasone. As used herein, the reduction in mortality, morbidity, viral load, or respiratory failure, days in ICU, days on mechanical ventilator, and the like means the reduction is in comparison to a subject (or subject population) treated with placebo. Likewise, any improvement, such as in WHO Ordinal Scale for Clinical Improvements, means an improvement in comparison to a subject (or subject population) treated with placebo.

Yet another embodiment of the invention, the methods further comprise at least one additional therapy. An embodiment of the method further comprises a second antiviral therapy such as a neuraminidase inhibitor, remdesivir, hydroxychloroquine, azithromycin, or hemagglutinin inhibitor. An embodiment of the method further comprises medications that modulate the immune system or host cell factors such as dexamethasone or another corticosteroid, an IL-6 inhibitor such as tocilizumab, interferons, an IL-1 inhibitor, or a kinase inhibitor such as baricitinib. Yet another embodiment of the invention, the methods further comprise an antibody therapy such as high titer COVID-19 convalescent plasma, intravenous immunoglobulin therapy (IVIG), a monoclonal antibody therapy such as casirivimab plus imdevimab, bamlanivimab, bamlanivimab plus etesevimab, tixagevimab plus cilgavimab (EUA December 2021), or bebtelovimab (EUA February 2022). An embodiment of the method further comprises an additional therapy such as nirmatrelvir plus ritonavir (EUA December 2021), or molnupiravir (EUA December 2021), or remdesivir and/or dexamethasone or other corticosteroids. An embodiment of the method further comprises an additional therapy such as tocilizumab. An embodiment of the method further comprises an additional therapy such as baricitinib. An embodiment of the method further comprises an additional therapy such as high titer COVID-19 convalescent plasma. An embodiment of the method further comprises an additional therapy such as IVIG. An embodiment of the method further comprises an additional therapy such as casirivimab plus imdevimab. An embodiment of the method further comprises an additional therapy such as bamlanivimab. An embodiment of the method further comprises an additional therapy such as bamlanivimab plus etesevimab. Yet another embodiment of the methods includes a second antiviral therapy that is at least one of favipiravir, lopinavir, ritonavir, nirmatrelvir plus ritonavir (EUA December 2021), molnupiravir (EUA December 2021), remdesivir, janus kinase inhibitors, hydroxychloroquine, azithromycin, amantadine, rimantadine, ribavirin, idoxuridine, trifluridine, vidarabine, acyclovir, ganciclovir, foscarnet, zidovudine, didanosine, peramivir, zalcitabine, stavudine, famciclovir, oseltamivir, zanamivir, or valaciclovir. Yet another embodiment of the methods includes a second therapy that is at least one of vitamins C or D, zinc, famotidine, ivermectin, or angiotensin converting enzyme inhibitor (ACEI) or angiotensin receptor binding (ARB) agent.

An embodiment of the invention encompasses methods wherein the compound of the invention is administered in an amount of about 1 mg to about 100 mg. Another embodiment of the invention encompasses methods wherein the compound of the invention is administered in an amount of about 4 to about 90 mg. Another embodiment of the invention encompasses methods wherein the compound of the invention is administered in an amount of about 9 mg to about 18 mg. Another embodiment of the invention encompasses methods wherein the compound of the invention is administered in an amount of about 9 mg. Another embodiment of the invention encompasses methods wherein the compound of the invention is administered in an amount of about 4 mg to about 45 mg. In yet another embodiment of the method encompasses at least one pharmaceutically acceptable excipient.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates the mean WHO Ordinal Scale for Clinical Improvement by Day (0=baseline). The area under the mean curve is 153 for the patient group treated with Compound 17ya and 182 for the group treated with placebo.

FIG. 2 illustrates the subgroup analysis of primary efficacy endpoint, relative risk of death point by day 60 (95% CI).

FIG. 3 illustrates the results of a subgroup analyses evaluating the relative risk of death in patients treated in Example 3 that were consistent with the overall study results favoring treating with Compound 17ya regardless of treatment received.

FIG. 4 illustrates the time to death (ITT population) in Study B.

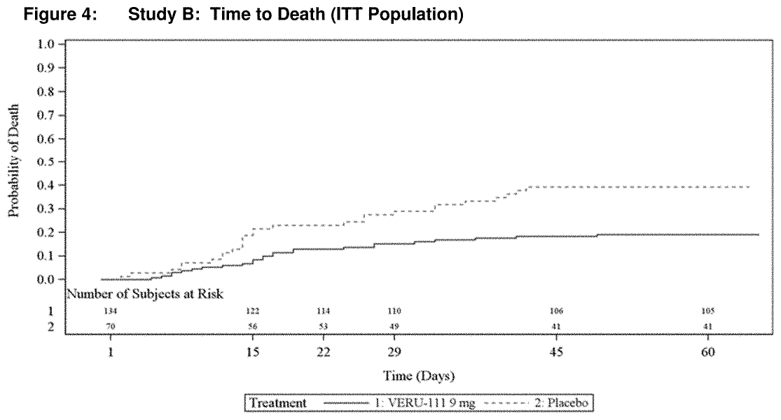

FIG. 5 illustrates a comparison of Phase 3 COVID-19 Studies with respect to mortality up to Day 30 as a function of Proportion of Patients with Severe Disease.

FIG. 6 illustrates for Study B, the time to death or dosing through nasogastric tube (Patients who started treatment orally in the intent-to-treat set).

### DETAILED DESCRIPTION OF THE INVENTION

Microtubule based macromolecule intracellular transport is a critical aspect of viral replication. For viral infection, expression of viral proteins alters the organization of these microtubular networks to serve their need to replicate and spread infectious virion. Microtubules not only facilitate infection, but microtubules are actively manipulated by viruses. Furthermore, cytoskeleton disruptor agents suppress viral infection.

Not to be limited by theory, the invention is based, in part, on the fact that tubulin interacts with the cytoplasmic domain of alphacoronavirus and betacoronavirus SARS-CoV spike S proteins. The reduction in infectious virus titer may follow by treatment with a drug that causes microtubule depolymerization, mainly because there is less S protein present at the assembly site due to impaired S protein-microtubule transport and that the incorporation process of S protein itself into virions is tubulin-dependent. Furthermore, disruption of microtubule trafficking impaired the egress out of the cell of these poorly assembled virions with less surface spike S proteins, making them less infectious. A microtubule depolymerizing agent may be effective in treating coronavirus infection by disrupting microtubule trafficking which is critical for the virus replication cycle.

The present invention is directed to antiviral therapy based upon the cytoskeleton disruptor activity of the claimed compounds that interrupts the intracellular microtubules trafficking network. Intended to overcome the disadvantages of the prior art, including but not limited to toxicity, the methods are directed to compounds specifically activated within virus-infected cell or within those cells that are preferably targeted by the virus. Not to be limited by theory, the invention is based upon virus reliance on the host cell machinery for successful replication. For instance, coronaviruses use the host secretory pathway during their replication cycle. The vesicular transport on secretory pathways is mostly mediated by microtubules and the corresponding motor proteins. The disruption of microtubules leads to decreased replication, reduced amount of released infectious particles, and decreased virus yield. Consequently, the virus load is reduced, thereby establishing an antiviral therapy. To address the need for novel, rapidly acting antiviral compounds, the inventors proposed a method of treating virus infections by the administration of the compounds described below.

In a particular embodiment, the compounds of the invention are orally bioavailable non-colchicine molecules that bind the “colchicine binding site” of α and β tubulin and inhibit tubulin polymerization at low nanomolar concentrations. These colchicine binding site inhibitors (CBSIs) have a broad scope of structures but generally possess predominantly indolyl, phenyl, or indazolyl A-rings (leftmost ring in Formula I), direct bond or amino linkers (X) between A- and B-rings, imidazole, or benzimidazole B-rings, methanone linkers (Y) between the B-ring and C-ring (rightmost ring in Formula I), and substituted phenyl C-rings. The compounds used in the methods are neither a substrate for MDRs including P-gp, MRPs, and BCRP, nor CYP3A4. The compounds used in the methods also decrease the transcription of βI, βIII, and βIV-tubulin isoforms (Li 2012). Further, the compounds used in the methods of the invention have good safety as they do not cause significant neurotoxicity, neutropenia, or myelosuppression and are well tolerated.

Further, the methods encompassed by the invention include compounds capable of influencing microtubule dynamics such that the compounds could be administered in sub-cytotoxic concentrations as systemic antiviral agents. This is in strong contrast to colchicine and other tubulin polymerization destabilizers used as antiviral drugs which possess high systemic toxicity and poor oral bioavailability.

The invention encompasses methods of treating a coronavirus infection in a subject in need thereof by administering to the subject a formulation having a therapeutically effective amount of a compound of Formula (I):

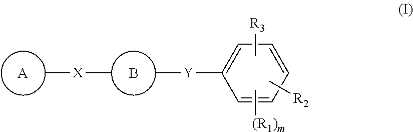

wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole, or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond, NH, —C═O, (C₁-C₄)alkyl, O, or S;
Y is a bond, —C═O, —C═S, SO₂, SO or S; and
m is 1-3, or a pharmaceutically acceptable salt, hydrate, polymorph, or isomer thereof.

The invention also encompasses methods of treating a coronavirus infection in a subject in need thereof by administering to the subject a compound of Formula (II) or a formulation having a therapeutically effective amount of a compound of Formula (II):

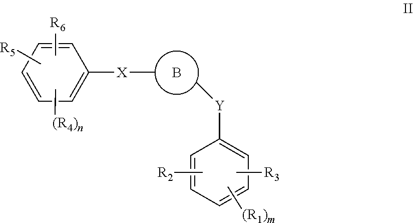

wherein
B is an imidazole, thiazole, or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₃, R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O;
n is 1-3; and
m is 1-3; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of Formula (III):

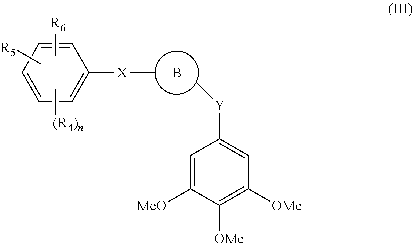

wherein
B is an imidazole, thiazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
n is 1-3; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of Formula (IV):

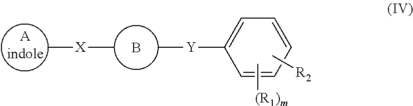

wherein
ring A is an indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁ and R₂ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-4; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of Formula IV(a):

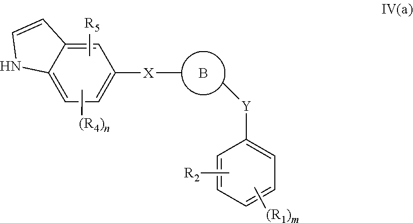

wherein
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
X is a bond or NH;
Y is —C═O;
n is 1-2; and
m is 1-4; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of Formula (V):

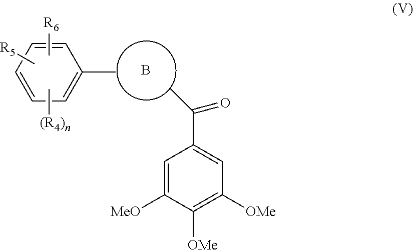

wherein
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
n is 1-3; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of the Formula (VI):

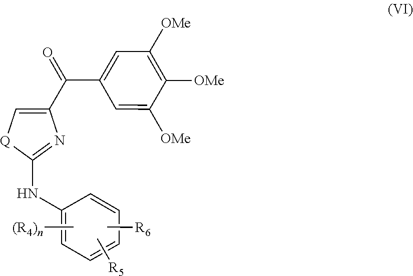

wherein
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is NH or S; and
n is 1-3; or a pharmaceutically acceptable salt, hydrate, polymorph, or isomer thereof.

Preferably, the variables for the compounds of Formula (VI) are R₄, R₅ and R₆ are independently hydrogen, (C₁-C₄)alkyl, halo(C₁-C₄)alkyl, O—(C₁-C₄)alkyl, O(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; Q is S or NH; and n is 1-3; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of the Formula VI in the following Table 1A:

TABLE 1A

| Formula VI | Compound | R₄ | R₅ | R₆ | Q |
|---|---|---|---|---|---|
| 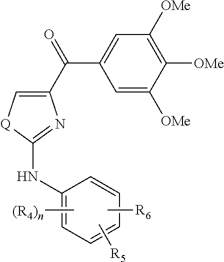 | 5e | H n = l | H | H | N |

The invention also encompasses methods of treating a coronavirus infection in a subject in need thereof by administering to the subject a formulation having a therapeutically effective amount of a compound of the Formula VII:

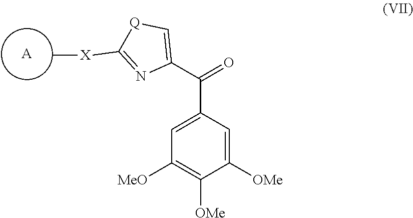

wherein
X is a bond, NH or S;
Q is NH or S; and
A is a phenyl, indolyl, or indazolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

Examples of compounds of Formula VII include, but are not limited to, (2-(phenylamino)-1H-imidazol-4-yl)(3,4,5-trimethoxyphenyl)methanone (5e), (2-(phenylamino)-1H-imidazol-4-yl)(3,4,5-trimethoxyphenyl)methanone hydrochloride salt (5He), and (2-(1H-indol-3-yl)-1H-imidazol-4-yl)(3,4,5-trimethoxyphenyl)methanone (17ya).

Preferably, the variables in the compounds of Formula VII are X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of the Formula VII(a):

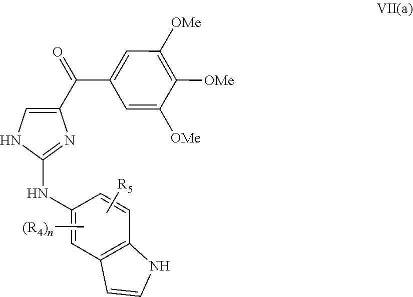

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of the Formula VII(b):

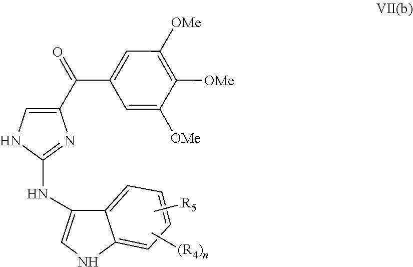

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating a coronavirus infection in a subject in need thereof by administering to the subject a formulation having a therapeutically effective amount of a compound of the Formula VII(c):

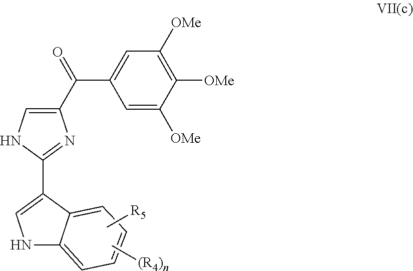

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof. Examples of compounds of Formula VII(c) include, but are not limited to, (2-(1H-indol-3-yl)-1H-imidazol-4-yl)(3,4,5-trimethoxyphenyl)methanone (17ya).

The invention also encompasses methods of treating a coronavirus infection in a subject in need thereof by administering to the subject a formulation having a therapeutically effective amount of a compound of the Formula 17ya:

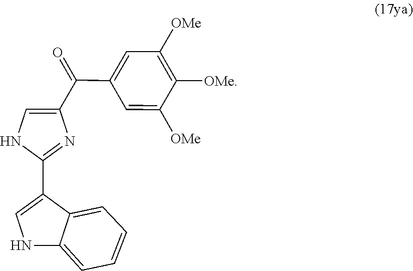 [SMILES: COc1cc(C(=O)c2c[nH]c(-c3c[nH]c4ccccc34)n2)cc(OC)c1OC]

The invention also encompasses methods of treating a coronavirus infection in a subject in need thereof by administering to the subject a formulation having a therapeutically effective amount of a compound of the Formula in the following Table 1B:

TABLE 1B

| Com- | |
|---|---|
| pound | Structure |
| 8 | 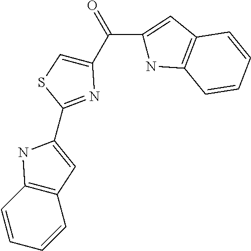 [SMILES: O=C(c1csc(-c2cc3ccccc3[nH]2)n1)c1cc2ccccc2[nH]1] |
| 9 | 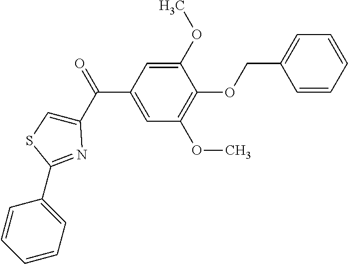 [SMILES: COc1cc(C(=O)c2csc(-c3ccccc3)n2)cc(OC)c1OCc1ccccc1] |
| 10 | 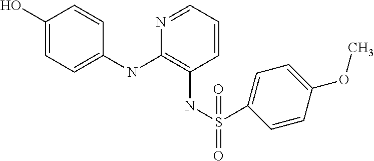 [SMILES: COc1ccc(S(=O)(=O)Nc2cccnc2Nc2ccc(O)cc2)cc1] |
| 11 | 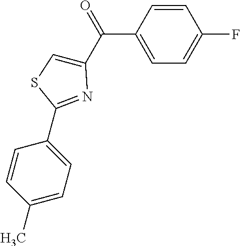 [SMILES: Cc1ccc(-c2nc(C(=O)c3ccc(F)cc3)cs2)cc1] |
| 12 | 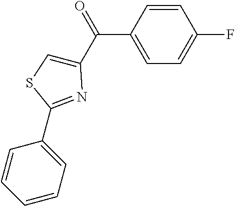 [SMILES: O=C(c1ccc(F)cc1)c1csc(-c2ccccc2)n1] |
| 13 | 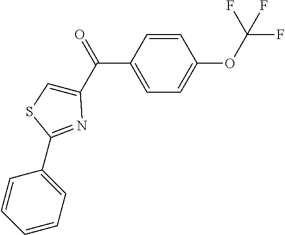 [SMILES: O=C(c1ccc(OC(F)(F)F)cc1)c1csc(-c2ccccc2)n1] |
| 14 | 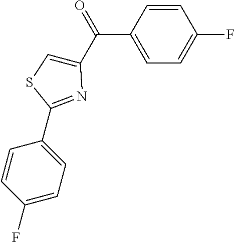 [SMILES: O=C(c1ccc(F)cc1)c1csc(-c2ccc(F)cc2)n1] |
| 16 | 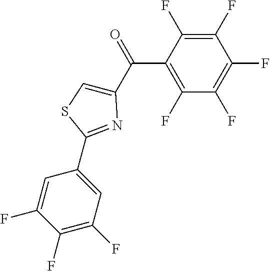 [SMILES: O=C(c1csc(-c2cc(F)c(F)c(F)c2)n1)c1c(F)c(F)c(F)c(F)c1F] |
| 17 | 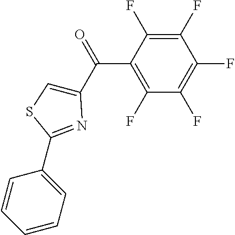 [SMILES: O=C(c1csc(-c2ccccc2)n1)c1c(F)c(F)c(F)c(F)c1F] |
| 18 | 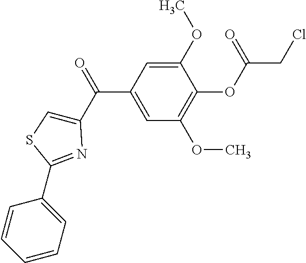 [SMILES: COc1cc(C(=O)c2csc(-c3ccccc3)n2)cc(OC)c1OC(=O)CCl] |
| 19 | 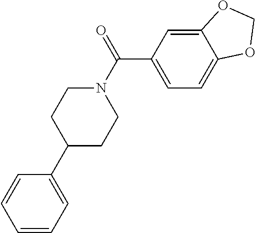 [SMILES: O=C(c1ccc2c(c1)OCO2)N1CCC(c2ccccc2)CC1] |
| 20 | 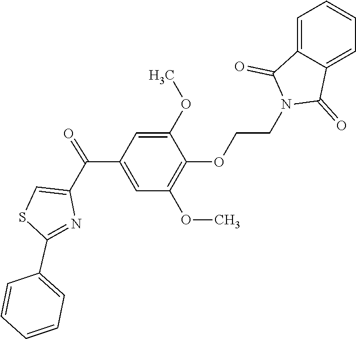 [SMILES: COc1cc(C(=O)c2csc(-c3ccccc3)n2)cc(OC)c1OCCN1C(=O)c2ccccc2C1=O] |
| 21 | 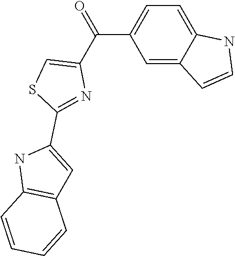 [SMILES: O=C(c1ccc2[nH]ccc2c1)c1csc(-c2cc3ccccc3[nH]2)n1] |
| 22 | 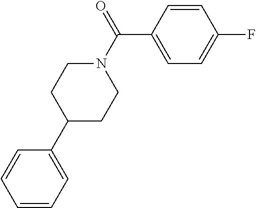 [SMILES: O=C(c1ccc(F)cc1)N1CCC(c2ccccc2)CC1] |
| 23 | 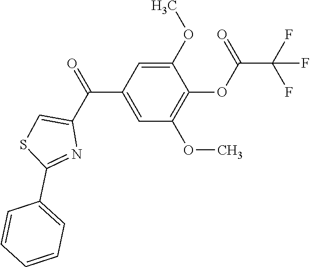 [SMILES: COc1cc(C(=O)c2csc(-c3ccccc3)n2)cc(OC)c1OC(=O)C(F)(F)F] |
| 24 | 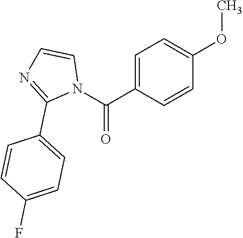 [SMILES: COc1ccc(C(=O)n2ccnc2-c2ccc(F)cc2)cc1] |
| 25 | 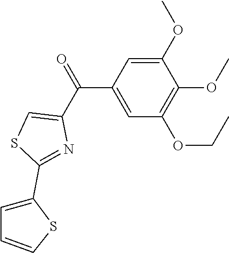 [SMILES: CCOc1cc(C(=O)c2csc(-c3cccs3)n2)cc(OC)c1OC] |
| 26 | 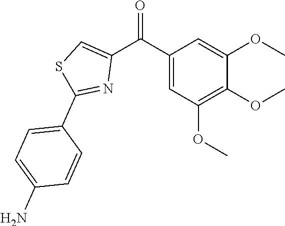 [SMILES: COc1cc(C(=O)c2csc(-c3ccc(N)cc3)n2)cc(OC)c1OC] |
| 27 | 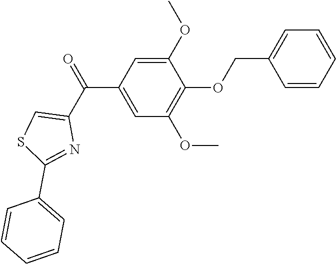 [SMILES: COc1cc(C(=O)c2csc(-c3ccccc3)n2)cc(OC)c1OCc1ccccc1] |
| 28 | 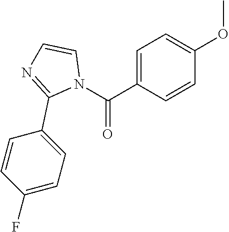 [SMILES: COc1ccc(C(=O)n2ccnc2-c2ccc(F)cc2)cc1] |
| 29 | 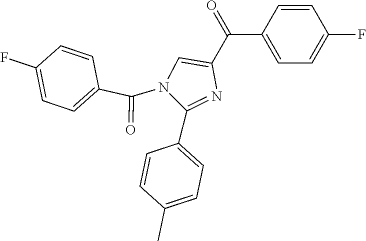 [SMILES: Cc1ccc(-c2nc(C(=O)c3ccc(F)cc3)cn2C(=O)c2ccc(F)cc2)cc1] |
| 30 | 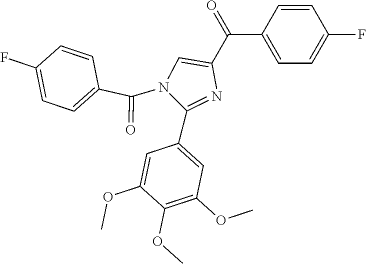 [SMILES: COc1cc(-c2nc(C(=O)c3ccc(F)cc3)cn2C(=O)c2ccc(F)cc2)cc(OC)c1OC] |
| 32 | 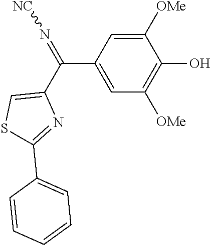 [SMILES: COc1cc(C(=NC#N)c2csc(-c3ccccc3)n2)cc(OC)c1O] |
| 33 | 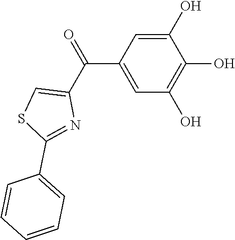 [SMILES: O=C(c1cc(O)c(O)c(O)c1)c1csc(-c2ccccc2)n1] |
| 34 | 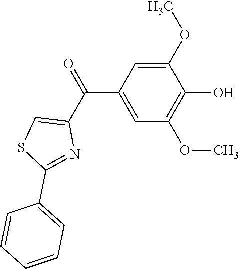 [SMILES: COc1cc(C(=O)c2csc(-c3ccccc3)n2)cc(OC)c1O] |
| 35 | 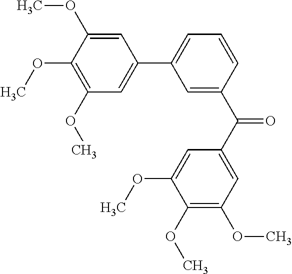 |
| 40 | 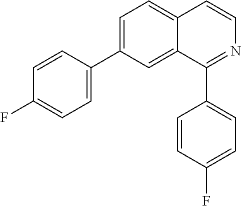 [SMILES: Fc1ccc(-c2ccc3ccnc(-c4ccc(F)cc4)c3c2)cc1] |
| 41 | 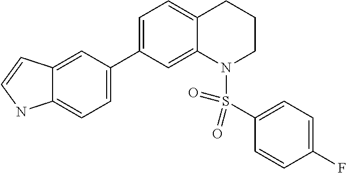 [SMILES: O=S(=O)(c1ccc(F)cc1)N1CCCc2ccc(-c3ccc4[nH]ccc4c3)cc21] |
| 42 | 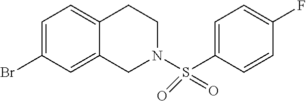 [SMILES: O=S(=O)(c1ccc(F)cc1)N1CCc2ccc(Br)cc2C1] |
| 43 | 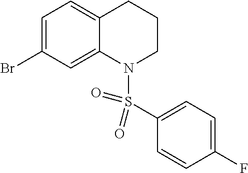 [SMILES: O=S(=O)(c1ccc(F)cc1)N1CCCc2ccc(Br)cc21] |
| 44 | 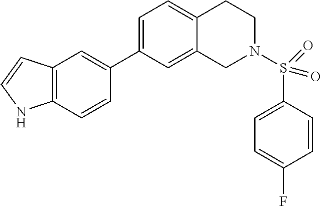 [SMILES: O=S(=O)(c1ccc(F)cc1)N1CCc2ccc(-c3ccc4[nH]ccc4c3)cc2C1] |
| 45 | 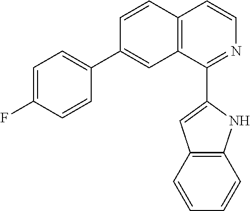 [SMILES: Fc1ccc(-c2ccc3ccnc(-c4cc5ccccc5[nH]4)c3c2)cc1] |
| 46 | 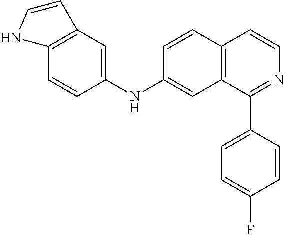 [SMILES: Fc1ccc(-c2nccc3ccc(Nc4ccc5[nH]ccc5c4)cc23)cc1] |
| 47 | 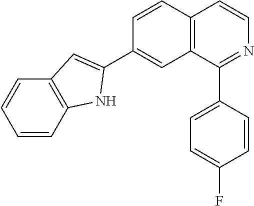 [SMILES: Fc1ccc(-c2nccc3ccc(-c4cc5ccccc5[nH]4)cc23)cc1] |
| 48 | 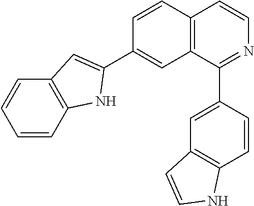 [SMILES: c1ccc2[nH]c(-c3ccc4ccnc(-c5ccc6[nH]ccc6c5)c4c3)cc2c1] |
| 49 | 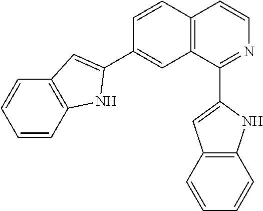 [SMILES: c1ccc2[nH]c(-c3ccc4ccnc(-c5cc6ccccc6[nH]5)c4c3)cc2c1] |
| 50 | 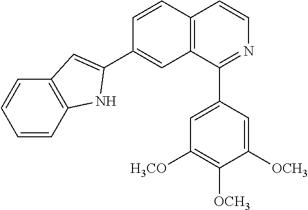 [SMILES: COc1cc(-c2nccc3ccc(-c4cc5ccccc5[nH]4)cc23)cc(OC)c1OC] |
| 51 | 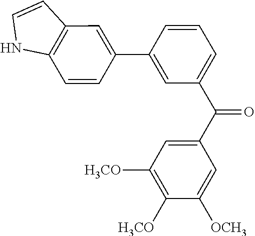 [SMILES: COc1cc(C(=O)c2cccc(-c3ccc4[nH]ccc4c3)c2)cc(OC)c1OC] |
| 52 | 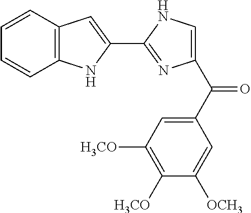 [SMILES: COc1cc(C(=O)c2c[nH]c(-c3cc4ccccc4[nH]3)n2)cc(OC)c1OC] |
| 53 | 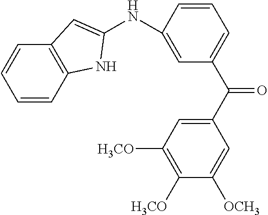 [SMILES: COc1cc(C(=O)c2cccc(Nc3cc4ccccc4[nH]3)c2)cc(OC)c1OC] |
| 54 | 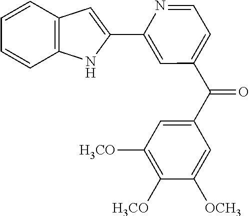 [SMILES: COc1cc(C(=O)c2ccnc(-c3cc4ccccc4[nH]3)c2)cc(OC)c1OC] |

The invention also encompasses methods of treating a coronavirus infection in a subject in need thereof by administering to the subject a formulation having a therapeutically effective amount of a compound of the Formula XIII:

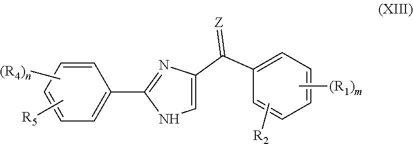

wherein
Z is O;
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer.

The invention also encompasses methods of treating a coronavirus infection in a subject in need thereof by administering to the subject a formulation having a therapeutically effective amount of a compound of the Formula XIV:

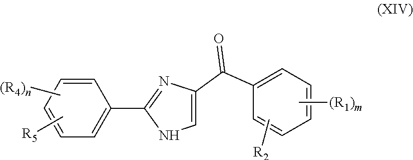

wherein R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO-alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer thereof.

Non limiting examples of compounds of formula XIV are selected from: (2-phenyl-1H-imidazol-4-yl)(3,4,5-trimethoxyphenyl)methanone (12aa), (4-fluorophenyl)(2-phenyl-1H-imidazol-4-yl)methanone (12af), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(4-hydroxy-3,5-dimethoxyphenyl)methanone (12fc), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(3,4-dimethoxyphenyl)-1H-imidazol-4-yl)(3,4,5-trimethoxyphenyl)methanone (12ha), (2-(4-(benzyloxy)phenyl)-1H-imidazol-4-yl)(4-fluorophenyl)methanone (12jb), (2-(4-bromophenyl)-1H-imidazol-4-yl)(3,4,5-trimethoxyphenyl)methanone (12la), and (2-(4-(trifluoromethyl)phenyl)-1H-imidazol-4-yl)(3,4,5-trimethoxyphenyl)methanone (12pa).

The invention also encompasses methods of treating a coronavirus infection in a subject in need thereof by administering to the subject a formulation having a therapeutically effective amount of a compound of the Formula XIVa:

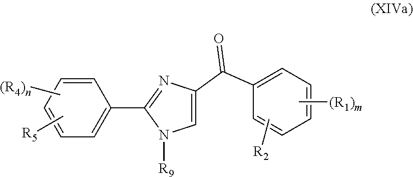

wherein R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₉ is H, linear or branched alkyl, aryl, CH₂Ph, benzyl, haloalkyl, aminoalkyl, OCH₂Ph, SO₂-Aryl, —(C═O)-Aryl or OH, optionally substituted with at least one of hydrogen, hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof;
m is an integer between 1-4; and
n is an integer between 1-4;
or a pharmaceutically acceptable salt, hydrate, polymorph, or isomer thereof.

Non limiting examples of compounds of formula XIVa are selected from: (4-fluorophenyl)(2-phenyl-1-(phenylsulfonyl)-1H-imidazol-4-yl)methanone (11af), (4-fluorophenyl)(2-(4-methoxyphenyl)-1-(phenylsulfonyl)-1H-imidazol-4-yl)methanone (11cb), (4-fluorophenyl)(1-(phenylsulfonyl)-2-(p-tolyl)-1H-imidazol-4-yl)methanone (11db), (2-(4-chlorophenyl)-1-(phenylsulfonyl)-1H-imidazol-4-yl)(4-fluorophenyl)methanone (11fb), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba), (1-benzyl-2-(p-tolyl)-1H-imidazol-4-yl)(3,4,5-trimethoxyphenyl)methanone (12daa), (1-methyl-2-(p-tolyl)-1H-imidazol-4-yl)(3,4,5-trimethoxyphenyl)methanone (12dab), and (4-fluorophenyl)(2-(4-methoxyphenyl)-1-methyl-1H-imidazol-4-yl)methanone (12cba).

The invention also encompasses methods of treating a coronavirus infection in a subject in need thereof by administering to the subject a formulation having a therapeutically effective amount of a compound of the Formula XV:

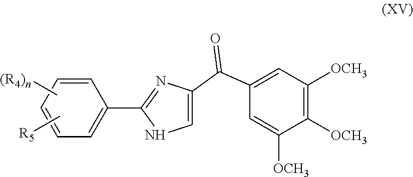

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Non limiting examples of compounds of formula XV are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (3,4,5-trimethoxyphenyl)(2-(3,4,5-trimethoxyphenyl)-1H-imidazol-4-yl)methanone (12ea), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(3,4-dimethoxyphenyl)-1H-imidazol-4-yl)(3,4,5-trimethoxyphenyl)methanone (12ha), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), and (2-(4-(trifluoromethyl)phenyl)-1H-imidazol-4-yl)(3,4,5-trimethoxyphenyl)methanone (12pa).

The invention also encompasses methods of treating a coronavirus infection in a subject in need thereof by administering to the subject a formulation having a therapeutically effective amount of a compound of the Formula XVI:

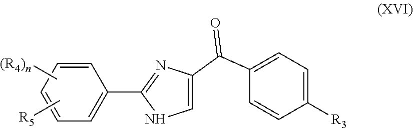

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₃ is I, Br, Cl, or F; and
n is 1-4; or a pharmaceutically acceptable salt, hydrate, polymorph, or isomer.

Non limiting examples of compounds of formula XVI are selected from: (4-fluorophenyl)(2-phenyl-1H-imidazol-4-yl)methanone (12af), (4-fluorophenyl)(2-(4-methoxyphenyl)-1H-imidazol-4-yl)methanone (12cb), (4-fluorophenyl)(2-(p-tolyl)-1H-imidazol-4-yl)methanone (12db), 4-fluorophenyl)(2-(3,4,5-trimethoxyphenyl)-1H-imidazol-4-yl)methanone (12eb), (2-(4-chlorophenyl)-1H-imidazol-4-yl)(4-fluorophenyl)methanone (12fb), (2-(4-(dimethylamino)phenyl)-1H-imidazol-4-yl)(4-fluorophenyl)methanone (12gb), or (2-(4-(benzyloxy)phenyl)-1H-imidazol-4-yl)(4-fluorophenyl)methanone (12jb).

The invention also encompasses methods of treating a coronavirus infection in a subject in need thereof by administering to the subject a formulation having a therapeutically effective amount of a compound of the Formula XVII:

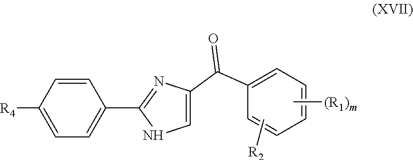

wherein R₄ is H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H;
wherein R₁ and R₂ are independently H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; and
m is 1-4; or a pharmaceutically acceptable salt, hydrate, polymorph, or isomer thereof.

Non limiting examples of compounds of formula XVII are selected from: (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(3,4,5-trihydroxyphenyl)methanone (13fa), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating a coronavirus infection in a subject in need thereof by administering to the subject a formulation having a therapeutically effective amount of a compound of the Formula XVII represented by the structure of formula 12fb:

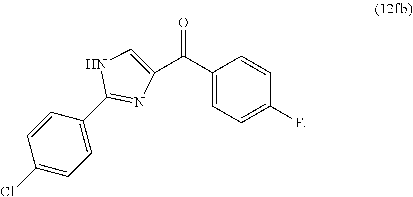 [SMILES: O=C(c1ccc(F)cc1)c1c[nH]c(-c2ccc(Cl)cc2)n1]

The invention also encompasses methods of treating a coronavirus infection in a subject in need thereof by administering to the subject a formulation having a therapeutically effective amount of a compound of the Formula XVII represented by the structure of formula 12cb:

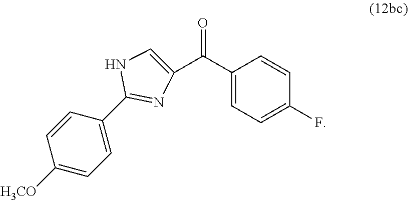 [SMILES: COc1ccc(-c2nc(C(=O)c3ccc(F)cc3)c[nH]2)cc1]

Non limiting examples of compounds are selected from: (4-methoxyphenyl)(2-phenyl-1H-imidazol-1-yl)methanone (12aba), (2-phenyl-1H-imidazol-1-yl)(3,4,5-trimethoxyphenyl)methanone (12aaa), 2-phenyl-1-(phenylsulfonyl)-1H-imidazole (10a), 2-(4-nitrophenyl)-1-(phenylsulfonyl)-1H-imidazole (10x), or 2-(4-(benzyloxy)phenyl)-1-(phenylsulfonyl)-1H-imidazole (10j).

The invention also encompasses methods of treating a coronavirus infection in a subject in need thereof by administering to the subject a formulation having a therapeutically effective amount of a compound of the Formula XIX:

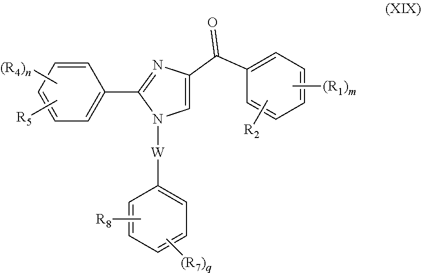

wherein
W is C═O, C═S, SO₂, or S═O;
R₁, R₄ and R₇ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂, R₅ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4;
n is 1-4; and
q is 1-4;
or its pharmaceutically acceptable salt, hydrate, polymorph, or isomer.

Non limiting examples of compounds of formula XIX are selected from: (2-(4-(dimethylamino)phenyl)-1-((4-methoxyphenyl)sulfonyl)-1H-imidazol-4-yl)(3,4,5-trimethoxyphenyl)methanone (11gaa), (2-(4-bromophenyl)-1-(phenylsulfonyl)-1H-imidazol-4-yl)(3,4,5-trimethoxyphenyl)methanone (11la), (4-fluorophenyl)(2-(4-methoxyphenyl)-1-(phenylsulfonyl)-1H-imidazol-4-yl)methanone (11cb), (2-(4-chlorophenyl)-1-(phenylsulfonyl)-1H-imidazol-4-yl)(4-fluorophenyl)methanone (11fb), (4-fluorophenyl)(2-phenyl-1-(phenylsulfonyl)-1H-imidazol-4-yl)methanone (11af), (4-fluorophenyl)(1-(phenylsulfonyl)-2-(p-tolyl)-1H-imidazol-4-yl)methanone (11db), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), or (2-(4-(dimethylamino)phenyl)-1-((4-methoxyphenyl)sulfonyl)-1H-imidazol-4-yl)(4-fluorophenyl)methanone (12gba).

The invention also encompasses methods of treating a coronavirus infection in a subject in need thereof by administering to the subject a formulation having a therapeutically effective amount of a compound of the Formula XIX represented by the structure of formula 11cb:

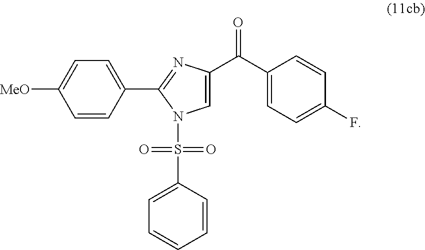 [SMILES: COc1ccc(-c2nc(C(=O)c3ccc(F)cc3)cn2S(=O)(=O)c2ccccc2)cc1]

The invention also encompasses methods of treating a coronavirus infection in a subject in need thereof by administering to the subject a formulation having a therapeutically effective amount of a compound of the Formula XIX represented by the structure of formula 11fb:

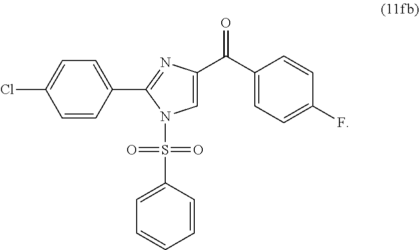 [SMILES: O=C(c1ccc(F)cc1)c1cn(S(=O)(=O)c2ccccc2)c(-c2ccc(Cl)cc2)n1]

The invention also encompasses methods of treating a coronavirus infection in a subject in need thereof by administering to the subject a formulation having a therapeutically effective amount of a compound of the Formula XX:

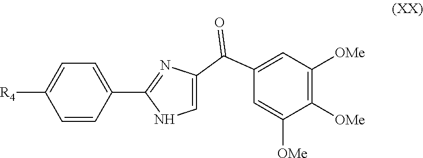

wherein
R₄ is independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer.

Non limiting examples of compounds of formula XX are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating a coronavirus infection in a subject in need thereof by administering to the subject a formulation having a therapeutically effective amount of a compound of the Formula XX represented by the structure of formula 12da:

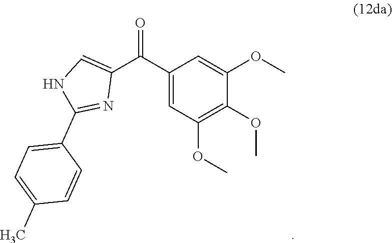 [SMILES: COc1cc(C(=O)c2c[nH]c(-c3ccc(C)cc3)n2)cc(OC)c1OC]

The invention also encompasses methods of treating a coronavirus infection in a subject in need thereof by administering to the subject a formulation having a therapeutically effective amount of a compound of the Formula XX represented by the structure of formula 12fa:

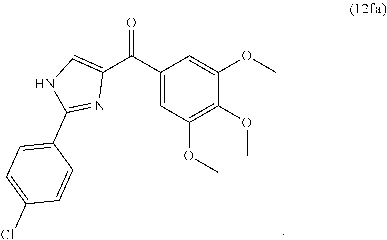 [SMILES: COc1cc(C(=O)c2c[nH]c(-c3ccc(Cl)cc3)n2)cc(OC)c1OC]

The invention also encompasses methods of treating a coronavirus infection in a subject in need thereof by administering to the subject a formulation having a therapeutically effective amount of a compound of the Formula XXI:

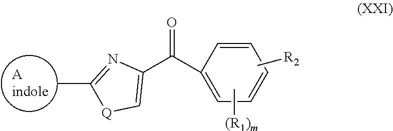

wherein
A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is NH or S;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
m is 1-4; or a pharmaceutically acceptable salt, hydrate, polymorph, or isomer thereof.

In one embodiment of the method, A ring of compound of formula XXI is substituted 5-indolyl. In another embodiment the substitution is —(C═O)-Aryl. In another embodiment, the aryl is 3,4,5-(OCH₃)₃-Ph. In another embodiment, A ring of compound of formula XXI is 3-indolyl. In another embodiment, A ring of compound of formula XXI is 5-indolyl. In another embodiment, A ring of compound of formula XXI is 2-indolyl. Non limiting examples of compounds of formula XXI are selected from: (5-(4-(3,4,5-trimethoxybenzoyl)-1H-imidazol-2-yl)-1H-indol-2-yl)(3,4,5-trimethoxyphenyl)methanone (15xaa); (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); (2-(1H-indol-3-yl)-1H-imidazol-4-yl)(3,4,5-trimethoxyphenyl)methanone (17ya); (2-(1H-indol-2-yl)thiazol-4-yl)(3,4,5-trimethoxyphenyl)methanone (62a); and (2-(1H-indol-5-yl)thiazol-4-yl)(3,4,5-trimethoxyphenyl)methanone (66a).

A particularly preferred method of treating a coronavirus infection of the invention uses at least one compound of formula XXI including (2-(1H-indol-1-yl)-1H-imidazol-4-yl)(3,4,5-trimethoxyphenyl)methanone; (2-(1H-indol-2-yl)-1H-imidazol-4-yl)(3,4,5-trimethoxyphenyl)methanone; (2-(1H-indol-3-yl)-1H-imidazol-4-yl)(3,4,5-trimethoxyphenyl)methanone (17ya); (2-(1H-indol-4-yl)-1H-imidazol-4-yl)(3,4,5-trimethoxyphenyl)methanone; (2-(1H-indol-5-yl)-1H-imidazol-4-yl)(3,4,5-trimethoxyphenyl)methanone; (2-(1H-indol-6-yl)-1H-imidazol-4-yl)(3,4,5-trimethoxyphenyl)methanone; or (2-(1H-indol-7-yl)-1H-imidazol-4-yl)(3,4,5-trimethoxyphenyl)methanone.

The invention also encompasses methods of treating a coronavirus infection in a subject in need thereof by administering to the subject a formulation having a therapeutically effective amount of a compound of the Formula XXIa:

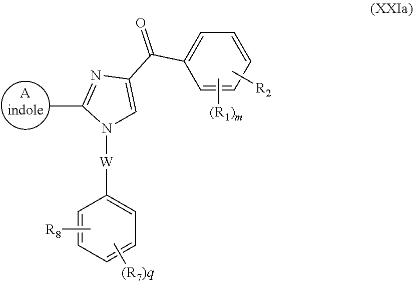

wherein
W is C═O, C═S, SO₂, or S═O;
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₇ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4; and
q is 1-4; or a pharmaceutically acceptable salt, hydrate, polymorph, or isomer thereof.

Non limiting examples of compounds of formula XXIa are selected from: (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); or (1-(phenylsulfonyl)-2-(1-(phenylsulfonyl)-1H-indol-3-yl)-1H-imidazol-4-yl)(3,4,5-trimethoxyphenyl)methanone (17yaa).

The invention also encompasses methods of treating a coronavirus infection in a subject in need thereof by administering to the subject a formulation having a therapeutically effective amount of a compound of the Formula XXII:

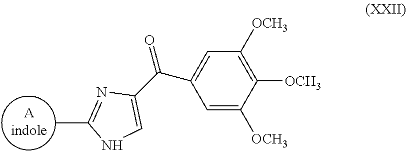

wherein
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
or a pharmaceutically acceptable salt, hydrate, polymorph, or isomer thereof.

In one embodiment of the method, A ring of compound of formula XXII is substituted 5-indolyl. In another embodiment the substitution is —(C═O)-Aryl. In another embodiment, the aryl is 3,4,5-(OCH₃)₃-Ph. In another embodiment, A ring of compound of formula XXII is 3-indolyl. Non limiting examples of compounds of formula XXII are selected from: (5-(4-(3,4,5-trimethoxybenzoyl)-1H-imidazol-2-yl)-1H-indol-2-yl)(3,4,5-trimethoxyphenyl)methanone (15xaa); and (2-(1H-indol-3-yl)-1H-imidazol-4-yl)(3,4,5-trimethoxyphenyl)methanone (17ya).

The invention also encompasses methods of treating a coronavirus infection in a subject in need thereof by administering to the subject a formulation having a therapeutically effective amount of a compound of the Formula XXI or XXII represented by the structure of formula 17ya:

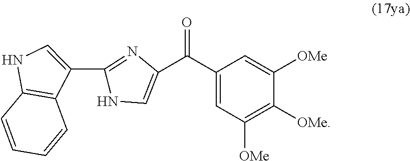 [SMILES: COc1cc(C(=O)c2c[nH]c(-c3c[nH]c4ccccc34)n2)cc(OC)c1OC]

In one embodiment of the method, R₄ and R₅ of compounds of formula XIII-XVI are hydrogens. Non-limiting examples of compounds of formula XIII-XVI wherein R₄ and R₅ are hydrogens are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); and (2-phenyl-1H-imidazol-4-yl)(m-tolyl)methanone (12ai). In an embodiment, W of compound of formula XIX is C═O. Non-limiting examples of compound of formula XIX wherein W is C═O are selected from (4-methoxyphenyl)(2-phenyl-1H-imidazol-1-yl)methanone (12aba) and (2-phenyl-1H-imidazol-1-yl)(3,4,5-trimethoxyphenyl)methanone (12aaa).

In one embodiment of the method, the compounds of this invention are the pure (E)-isomers. In another embodiment, the compounds of this invention are the pure (Z)-isomers. In another embodiment, the compounds of this invention are a mixture of the (E) and the (Z) isomers. In one embodiment, the compounds of this invention are the pure (R)-isomers. In another embodiment, the compounds of this invention are the pure (S)-isomers. In another embodiment, the compounds of this invention are a mixture of the (R) and the (S) isomers.

The compounds of the present invention can also be present in the form of a racemic mixture, containing substantially equivalent amounts of stereoisomers. In another embodiment, the compounds of the present invention can be prepared or otherwise isolated, using known procedures, to obtain a stereoisomer substantially free of its corresponding stereoisomer (i.e., substantially pure). As used herein, the term “substantially pure” refers to stereoisomer is at least about 95% pure in one isomer. Alternatively, the stereoisomer purity may be at least about 98% pure, and more preferably at least about 99% pure.

Compounds can also be in the form of a hydrate, which means that the compound further includes a stoichiometric or non-stoichiometric amount of water bound by non-covalent intermolecular forces.

The invention includes “pharmaceutically acceptable salts” of the compounds used in the method of the invention, which may be produced, by reaction of a compound of this invention with an acid or base. Certain compounds, particularly those possessing acid or basic groups, can also be in the form of a salt, preferably a pharmaceutically acceptable salt. As used herein, the term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically-acceptable salts of amines of compounds used in the method of the invention may be prepared from an inorganic acid or from an organic acid. In one embodiment, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

Examples of organic salts of amines include, but are not limited to, aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

Examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

Examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

Typical salts include, but are not limited to, hydrofluoric, hydrochloric, hydrobromic, hydroiodic, boric, nitric, perchloric, phosphoric, sulfuric, acetate, citrate, maleate, malate, or mesylate. Preferred salts include hydrofluoric, hydrochloric, hydrobromic, hydroiodic, acetate, citrate, maleate, or mesylate. More preferred salts include hydrochloric, acetate, or maleate.

The salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

The compounds used in the methods of the invention were synthesized using the methodology described in U.S. Pat. Nos. 8,592,465; 8,822,513; 9,029,408; 9,334,242; 9,447,049; 10,301,285; and 11,084,811, hereby incorporated by reference.

### Pharmaceutical Composition

The methods of the invention include the administration of a pharmaceutical composition including a pharmaceutically acceptable carrier and at least one compound described herein. Typically, the pharmaceutical composition may include a compound or its pharmaceutically acceptable salt, and at least one pharmaceutically acceptable excipient. The term “pharmaceutically acceptable excipient” refers to any suitable adjuvants, carriers, excipients, flavorant, or stabilizers, and can be used in pharmaceutical formulations either in solid or liquid form. Such forms include, but are not limited to, tablets, capsules, powders, solutions, suspensions, or emulsions.

The amount of compound used in the method and the dosage regimen for treating a disease condition depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods.

Typically, the formulations have from about 0.01 to about 99 percent by weight of at least one compound by weight, preferably from about 20 to 75 percent of active compound(s), together with the adjuvants, carriers and/or excipients. While individual needs may vary, determination of optimal ranges of effective amounts of each component is within the skill of the art. Typical daily dosages include about 2 mg to about 200 mg or about 1 mg to about 100 mg, preferred daily dosages include about 4 mg to about 90 mg, and the most preferred dosages include about 4 mg to about 80 mg of the compound. Other preferred dosages include the antiviral compound in an amount of about 4 mg to about 45 mg, or about 9 mg to about 18 mg. Alternatively, a dose is from about 0.01 mg to 150 mg/kg body weight, preferably from about 1 mg to about 100 mg/kg body weight, and more preferably from about 2 to 50 mg/kg body weight, may be appropriate. The daily dose can be administered in one to four doses per day. Treatment regimen for the administration of the compounds of the present invention can also be determined readily by those with ordinary skill in art. That is, the frequency of administration and size of the dose can be established by routine optimization, preferably while minimizing any side effects.

Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a subject's condition, a maintenance dose of a compound, composition or formulation may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

The methods may include “additional therapeutic agents” including, but are not limited to, immune therapies (e.g., interferon), therapeutic vaccines, antifibrotic agents, anti-inflammatory agents such as corticosteroids or NSAIDs, bronchodilators such as beta-2 adrenergic agonists and xanthines (e.g., theophylline), mucolytic agents, anti-muscarinics, anti-leukotrienes, inhibitors of cell adhesion (e.g., ICAM antagonists), anti-oxidants (e.g., N-acetylcysteine), cytokine agonists, cytokine antagonists, lung surfactants and/or antimicrobial and anti-viral agents (e.g., ribavirin and amantadine). The methods of the invention may also be used in combination with gene replacement therapy.

The methods of the invention may be administered in conjunction with other antiviral therapies to treat the infection or disease associated with the coronavirus infection, e.g., combination therapy. Suitable antiviral agents contemplated for use in combination with the methods of the invention may include nucleoside and nucleotide reverse transcriptase inhibitors (NRTIs), non-nucleoside reverse transcriptase inhibitors (NNRTIs), protease inhibitors and other antiviral drugs. Examples of suitable NRTIs include zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-I0652; emtricitabine [(−)-FTC]; beta-L-FD4 (also called beta-L-D4C and named beta-L-2′,3′-dicleoxy-5-fluoro-cytidene); DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfinavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

Other antiviral agents include, but are not limited to, neuraminidase inhibitors, hemagglutinin inhibitor, hydroxychloroquine, azithromycin, or medications that modulate the immune system or host cell factors such dexamethasone. Examples include, but are not limited to, favipiravir, lopinavir, ritonavir, remdesivir, janus kinase inhibitors, hydroxychloroquine, azithromycin, amantadine, rimantadine, ribavirin, idoxuridine, trifluridine, vidarabine, acyclovir, ganciclovir, foscarnet, zidovudine, didanosine, peramivir, zalcitabine, stavudine, famciclovir, oseltamivir, zanamivir, and valaciclovir. An embodiment of the method further comprises an additional therapy such as a remdesivir and/or dexamethasone. An embodiment of the method further comprises an additional therapy such as casirivimab plus imdevimab. An embodiment of the method further comprises an additional therapy such as bamlanivimab. An embodiment of the method further comprises an additional therapy such as molnupiravir. An embodiment of the method further comprises an additional therapy such as nirmatrelvir plus ritonavir.

The methods of treating coronavirus infections may further comprise other therapies. For example, the methods may include a second antiviral therapy such as a neuraminidase inhibitor, remdesivir, hydroxychloroquine, azithromycin, or hemagglutinin inhibitor. Other therapies included in the methods are medications that modulate the immune system or host cell factors such as dexamethasone; corticosteroids; an IL-6 inhibitor such as tocilizumab; interferons; an IL-1 inhibitor; or a kinase inhibitor such as baricitinib. The methods may further comprise an antibody therapy such as high titer COVID-19 convalescent plasma, IVIG, a monoclonal antibody therapy such as casirivimab plus imdevimab, bamlanivimab, or bamlanivimab plus etesevimab. The methods may further comprise tocilizumab or baricitinib. The methods may further comprise an additional therapy such as high titer COVID-19 convalescent plasma; IVIG; casirivimab plus imdevimab; bamlanivimab; or bamlanivimab plus etesevimab. The methods may include a second antiviral therapy that is at least one of favipiravir, lopinavir, ritonavir, remdesivir, janus kinase inhibitors, hydroxychloroquine, azithromycin, amantadine, rimantadine, ribavirin, idoxuridine, trifluridine, vidarabine, acyclovir, ganciclovir, foscarnet, zidovudine, didanosine, peramivir, zalcitabine, stavudine, famciclovir, oseltamivir, zanamivir, or valaciclovir. The methods may include a second therapy that is at least one of vitamins C or D, zinc, famotidine, ivermectin, or angiotensin converting enzyme inhibitor (ACEI) or angiotensin receptor binding (ARB) agent.

The solid unit dosage forms can be of the conventional type. The solid form can be a capsule and the like, such as an ordinary gelatin type containing the compounds and a carrier. Carriers include, but are not limited to, lubricants and inert fillers such as, castor oil and similar materials, lactose, sucrose, or cornstarch. The formulations may be tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

The invention can be mixed at cold temperatures, room temperature, or elevated temperatures with a liquid carrier such as a fatty oil, castor oil, or other similar oil to manufacture tablets, capsules, and the like.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, the formulation may include excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Typical compositions according to the present invention are prepared so that an oral dosage unit contains between about 1 mg and 100 mg of active compound, and preferred oral compositions contain between 1 mg and 50 mg of active compound.

The formulations may be orally administered with an inert diluent, or with an assimilable edible carrier, or they can be enclosed in hard or soft shell capsules, or they can be compressed into tablets, or they can be incorporated directly with the food of the diet. A preferred formulation is an oral formulation.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions used in the method of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

The formulation may also be administered parenterally. Solutions or suspensions of these formulations can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the formulations may be in solution or suspension may be packaged in a pressurized aerosol container together with suitable propellants, for example, hydrocarbon propellants like propane, butane, or isobutane with conventional adjuvants. The formulations also may be administered in a non-pressurized form such as in a nebulizer or atomizer.

When administering the formulations in the methods of the invention, the formulations may be administered systemically or sequentially. Administration can be accomplished in any manner effective for delivering the compounds or the pharmaceutical compositions to the site of viral infection. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

The invention is directed to methods of treating coronavirus infections and anti-viral formulations with the compounds and formulations described above. The compounds and formulations thereof have utility in treating viral infections by disrupting microtubule polymerization. The formulations may optionally comprise additional active ingredients, whose activity is useful for treating coronavirus viral infections, treat adverse effect associated with the compounds or dosages of a particular formulation, and/or delay or extend the release of the ingredients. The indirect antivirals agents of this invention bind to a conserved host target which also suggests broad antiviral efficacy across diverse families of viruses as has been demonstrated for members of the coronavirus pathogenic viruses. An advantage of indirect antiviral agents regarding the lack of selective pressures with their use is operative regardless of whether the viral infection is an SARS-CoV, MERS-CoV, or SARS-CoV-2 generally. Nor does evolution of the coronavirus alter interaction of the indirect anti-viral with its target. Accordingly, known variants of SAR-CoV-2 such as alpha, beta, delta, omicron, has been demonstrated to be susceptible to the colchicine binding site inhibitors (CBSI) of this invention. Further, future variants will be susceptible to the CBSI of this invention as there are not redundant intracellular trafficking systems for coronavirus, nor are alternative bindings sites to microtubulines known for coronaviruses.

In particular, the methods of the invention may be used to treat infections caused by viruses including those of the superfamilies of Coronaviridae. Also, the methods of the invention may be used to treat infections caused by viruses including, but not limited to, SARS, MERS-CoV, and COVID-19. Preferably, the methods of the invention treat viral infections caused by SARS-CoV, MERS-CoV, or COVID-19. More preferably, the methods of the invention treat viral infections caused by COVID-19 (SARS-CoV-2). The methods of the invention may be used to treat all SARS-CoV-2 variants such as alpha, beta, gamma, delta, omicron including sub-lineages such as BA.1 and BA.2 as demonstrated in phase II and phase III trials, and other variants that will emerge (descendent lineages).

The methods of the invention may be used to treat infections caused by SARS-CoV, MERS-CoV, or SARS-CoV-2, and in particular SARS-CoV-2 infection. The methods of the invention may be used to treat subjects with SARS-CoV-2 infection at high risk for acute respiratory distress syndrome (ARDS) or severe acute respiratory syndrome (SARS). One embodiment of the invention encompasses methods wherein treating a subject with SARS-CoV-2 infection reduces mortality. Another embodiment of the invention encompasses methods wherein treating a subject with SARS-CoV-2 infection at high risk for acute respiratory distress syndrome (ARDS) or severe acute respiratory syndrome (SARS) reduces mortality. Another embodiment of the invention encompasses methods wherein treating a subject with SARS-CoV-2 infection at high risk for acute respiratory distress syndrome (ARDS) or severe acute respiratory syndrome (SARS) reduces viral load. Another embodiment of the invention encompasses methods wherein treating a subject with SARS-CoV-2 infection reduces morbidity. Another embodiment of the invention encompasses methods wherein treating a subject with SARS-CoV-2 infection at high risk for acute respiratory distress syndrome (ARDS) or severe acute respiratory syndrome (SARS) reduces morbidity. Another embodiment of the invention encompasses methods wherein treating a subject with SARS-CoV-2 infection reduces morbidities including atrial fibrillation, bradycardia, pneumonia, bacterial pneumonia, hyperkalemia, hypokalemia, hypophosphatemia, chronic bronchitis, hypoxia, pneumothorax, respiratory failure, acute renal injury, cardiac arrest, septic shock, or hypotension, or any combination thereof. Another embodiment of the invention encompasses methods wherein treating a subject with SARS-CoV-2 infection at high risk for acute respiratory distress syndrome (ARDS) or severe acute respiratory syndrome (SARS) reduces morbidities including atrial fibrillation, bradycardia, pneumonia, bacterial pneumonia, hyperkalemia, hypokalemia, hypophosphatemia, chronic bronchitis, hypoxia, pneumothorax, respiratory failure, acute renal injury, cardiac arrest, septic shock, or hypotension, or any combination thereof. Another embodiment of the invention encompasses methods wherein treating a subject with SARS-CoV-2 infection reduces morbidities including respiratory failure, acute renal injury, cardiac arrest, septic shock, or hypotension, or any combination thereof. Another embodiment of the invention encompasses methods wherein treating a subject with SARS-CoV-2 infection at high risk for acute respiratory distress syndrome (ARDS) or severe acute respiratory syndrome (SARS) reduces morbidities including respiratory failure, acute renal injury, cardiac arrest, septic shock, or hypotension, or any combination thereof.

Another embodiment of the invention encompasses methods wherein treating a subject with SARS-CoV-2 infection reduces respiratory failure, days in ICU, days on mechanical ventilator, or improves WHO Ordinal Scale for Clinical Improvements. Another embodiment of the invention encompasses methods wherein treating a subject with SARS-CoV-2 infection at high risk for acute respiratory distress syndrome (ARDS) or severe acute respiratory syndrome (SARS) reduces respiratory failure, days in ICU, days on mechanical ventilator, days in the hospital, or improves WHO Ordinal Scale for Clinical Improvements. Another embodiment of the invention encompasses methods wherein treating a subject with SARS-CoV-2 infection reduces days in the mechanical ventilation. Another embodiment of the invention encompasses methods wherein treating a subject with SARS-CoV-2 infection reduces days in the ICU. Another embodiment of the invention encompasses methods wherein treating a subject with SARS-CoV-2 infection reduces days in the hospital. Another embodiment of the invention encompasses methods wherein treating a subject with SARS-CoV-2 infection reduces mortality. Another embodiment of the invention encompasses methods wherein treating a subject with SARS-CoV-2 infection improves WHO Ordinal Scale for Clinical Improvements of the subject. Another embodiment of the invention encompasses methods wherein treating a subject with SARS-CoV-2 infection reduces morbidity. Another embodiment of the invention encompasses methods wherein treating a subject with SARS-CoV-2 infection reduces days on the mechanical ventilator, days in the ICU, days in the hospital, mortality, morbidity, or improves WHO Ordinal Scale for Clinical Improvements, or any combination thereof.

Another embodiment of the invention encompasses methods wherein treating a subject with SARS-CoV-2 infection at high risk for acute respiratory distress syndrome (ARDS) or severe acute respiratory syndrome (SARS) reduces days in the mechanical ventilation. Another embodiment of the invention encompasses methods wherein treating a subject with SARS-CoV-2 infection at high risk for acute respiratory distress syndrome (ARDS) or severe acute respiratory syndrome (SARS) reduces days in the ICU. Another embodiment of the invention encompasses methods wherein treating a subject with SARS-CoV-2 infection at high risk for acute respiratory distress syndrome (ARDS) or severe acute respiratory syndrome (SARS) reduces days in the hospital. Another embodiment of the invention encompasses methods wherein treating a subject with SARS-CoV-2 infection at high risk for acute respiratory distress syndrome (ARDS) or severe acute respiratory syndrome (SARS) reduces mortality. Another embodiment of the invention encompasses methods wherein treating a subject with SARS-CoV-2 infection at high risk for acute respiratory distress syndrome (ARDS) or severe acute respiratory syndrome (SARS) improves WHO Ordinal Scale for Clinical Improvements. Another embodiment of the invention encompasses methods wherein treating a subject with SARS-CoV-2 infection at high risk for acute respiratory distress syndrome (ARDS) or severe acute respiratory syndrome (SARS) reduces morbidity. Another embodiment of the invention encompasses methods wherein treating a subject with SARS-CoV-2 infection at high risk for acute respiratory distress syndrome (ARDS) or severe acute respiratory syndrome (SARS) reduces days on the mechanical ventilator, days in the ICU, days in the hospital, mortality, morbidity, or improves WHO Ordinal Scale for Clinical Improvements, or any combination thereof.

Another embodiment of the invention encompasses methods wherein treating a subject with SARS-CoV-2 infection reduces mortality or respiratory failure in subjects >60 years of age. Another embodiment of the invention encompasses methods wherein treating a subject with SARS-CoV-2 infection at high risk for acute respiratory distress syndrome (ARDS) or severe acute respiratory syndrome (SARS) reduces mortality or respiratory failure in subjects >60 years of age. Another embodiment of the invention encompasses methods wherein treating a subject with SARS-CoV-2 infection reduces mortality or respiratory failure when dosed in combination with remdesivir and/or dexamethasone. Another embodiment of the invention encompasses methods wherein treating a subject with SARS-CoV-2 infection at high risk for acute respiratory distress syndrome (ARDS) or severe acute respiratory syndrome (SARS) reduces mortality or respiratory failure when dosed in combination with remdesivir and/or dexamethasone.

The invention encompasses methods for treating coronavirus infections in a subject in need thereof comprising administering to the subject a formulation having a compound described herein or a pharmaceutically acceptable salt, hydrate, polymorph, or isomer thereof in a therapeutically effective amount to treat the coronavirus infection. The methods include at least one of compound 12db, compound 11cb, compound 11fb, compound 12da, compound 12fa, compound 12fb, compound 12cb, compound 55, compound 66a, or compound 17ya. In a particular method, the method includes compound 17ya.

As used herein unless otherwise stated, the term “subject” or “patient” refers to any mammalian patient, including without limitation, humans, other primates, dogs, cats, horses, cows, sheep, pigs, bats, rats, mice, and other rodents. In particular, the subject is a human, and alternatively may be only male or only female.

When administering the compounds and formulations described herein, the formulations can be administered systemically or directly to a specific site where the viral infection is present. Administration may be accomplished in any manner effective for delivering the compounds or the pharmaceutical compositions to the viral infection site. Administration methods include, but are not limited to, oral, topical, transdermal, parenteral, subcutaneous, intravenous, intramuscular, intraperitoneal, intranasal, by intracavitary or intravesical instillation, intraocular, intraarterial, intralesional, or by application to the mucous membrane. Mucous membranes include those found in the nose, throat, and/or bronchial tubes, among others. Preferably, the formulation is administered orally. Administration may be simultaneous or sequential with additional antiviral compounds or formulations, or treatments used to address side effects associated with the compounds or dosages.

Treatment of COVID-19 with compound 17ya had significant biological advantages over treatment with placebo. For example, in the Phase II clinical trial (Example 1) at least about 30% to about 100% of compound 17ya treated patients were kept alive without respiratory failure (primary endpoint) versus the patients treated with “placebo” (existing standard of care—remdesivir, dexamethasone, convalescent plasma, etc.). For example, compound 17ya reduced the proportion of patients who died up to 60 days after initiation of treatment from 30% (6/20) in the placebo group to 5% (1/19) after treatment with compound 17ya. The mortality reduction is about 82% reduction in the compound 17ya treated group. Therefore, treatment with compound 17ya is expected to reduce death by about 30% to about 100% of the treated group as compared to the group treated with placebo. During the study, treatment failures (defined as death or respiratory failure) were 35% in the placebo group while the treatment failures were reduced to 15.8% in group treated with compound 17ya after 15 days of treatment. The numbers reduction improved as treatment continued where treatment failures were about 30% in the placebo treated group to 10.5% in the compound 17ya treated group after 29 days on study. The results represent a 55% reduction in treatment failures after 15 days of treatment and a 65% reduction in treatment failures after 29 days on study with compound 17ya when compared to the placebo treated group. Thus, it is expected that compound 17ya will reduce treatment failures by about 30% to about 100% during treatment. Other measures of success were observed on Covid-19 treated patients.

Treatment with compound 17ya reduced the days on mechanical ventilation from an average of 5.4 days in the placebo group to 1.6 days in the group treated with compound 17ya. Those treated with placebo had about a 3.4-fold increase in days on mechanical ventilation compared to the compound 17ya treated group. Consequently, it is expected that treatment with compound 17ya will reduce the days on mechanical ventilation by about 30% to about 100% as compared to the patients treated with placebo. Another reduction was observed with patients treated with compound 17ya with regard to the days spent in ICU. The placebo treated group spent an average of 9.6 days in ICU, while those treated with compound 17ya spent about 3 days in the ICU. The placebo treated group spent an additional 3.2-fold more days in the ICU, in contrast to those patients treated with compound 17ya. Therefore, treatment with compound 17ya is expected to reduce the days spent in ICU by about 30% to about 100%.

The study sponsor (Veru) has conducted post-hoc, sub-group analyses of the data from the phase II study. The following additional observations are made from this study: (1) In the compound 17ya treated group there was one patient who was noncompliant with oxygen supplementation. This patient noncompliant with standard of care in this study. An analysis of the primary endpoint excluding this patient (MITT population) from the analysis shows a 30% failure rate in the Placebo group (same as Table 2) compared to a 5.6% failure rate in the compound 17ya treated group at Day 29 (lower than in Table 2). This represents an 81% reduction in treatment failures. (2) It is well recognized that older patients are at higher risk for death and respiratory failure in patients with COVID-19 compared to younger patients. In an analysis of treatment failures in patients >60 years of age showed that a statistically significant (p-value of 0.0456 (chi-square)) and clinically meaningful reduction in treatment failures were observed in the compound 17ya treated (1/11 or 9%) group compared to placebo (4/8 or 50%) in this high-risk population. (3) A risk factor for an adverse clinical outcome in a patient with COVID-19 is the severity of disease at presentation. To assess this risk factor, an analysis of patients with a WHO Score of Disease Severity ≥5 at baseline was performed. The outcome of this analysis shows a clinically meaningful reduction (78%) in mortality were observed in the compound 17ya treated (1/10 or 10%) group compared to placebo (6/13 or 46%) in this high-risk population. (4) An analysis of the days in ICU in evaluable patients showed a statistically significant (p-value of 0.0469 (t-test)) and clinically meaningful reduction in days in ICU in the compound 17ya treated (3 days; N=18 subjects) group compared to placebo (9.55 days; N=20 days). (5) Additionally, the proportion of patients that were in the ICU for ≥3 days on study is statistically significantly higher (p-value of 0.0390 (chi-square)) in the placebo group (11/20 or 55%) compared to the compound 17ya treated (4/18 or 22%) group. (6) In this study, patients were permitted to receive standard of care. At the time of the study, the standard of care included treatment with remdesivir and/or dexamethasone under an Emergency Use Authorization. There were 11 patients in the study that did not receive either remdesivir or dexamethasone (6 in the compound 17ya treated group and 5 in the placebo group). An analysis of patients that received the recognized standard of care was conducted. Specifically, the days in ICU and the days on mechanical ventilation were compared between the treatment groups. In this population, in patients that received standard of care, no patient treated with compound 17ya required admission in the ICU or mechanical ventilation and there were no mortalities in this patient group. In the placebo group, 53% (8/16) required ICU admission with an average of 9.5 days in the ICU, 20% (3/15) required mechanical ventilation with an average of 3.9 days of mechanical ventilation, and 27% (4/15) died on study.

Overall, the study sponsor proposes that compound 17ya shows strong clinically meaningful outcomes in this small, proof-of-concept, Phase 2 study with statistically significant observations in reductions in death in the ITT population and in post-hoc, high-risk sub-group analyses, and days in ICU. It is important to note that all the parameters measured in the study show clinically meaningful outcomes with compound 17ya compared to placebo and there are no parameters that do not indicate benefit with compound 17ya treatment compared to placebo although some parameters do not reach statistical significance in this small study.

Safety: The overall safety conclusions are: (1) There were no treatment related serious adverse events observed on the study; and (2) there were no treatment related adverse events observed on the study. The treatment emergent adverse events that were observed in at least 2 patients in either treatment group in the study are presented in Example 1. The treatment emergent serious adverse events observed in the study are also presented in Example 1. There is no imbalance against compound 17ya in serious adverse events observed in the study. Overall, compound 17ya was well tolerated in this patient population with no clinically relevant safety observations in the compound 17ya treated group.

The use of remdesivir and dexamethasone did not have a significant effect on patient outcomes in the study. “Significant outcome” for the purposes of the clinical trial above would be reduction in treatment failures (death or respiratory failure), increase in treatment success (alive without respiratory failure), decrease in death (all-cause mortality), decrease in days in ICU, decrease in days on mechanical ventilation, or decrease in subjects requiring mechanical ventilation, or possibly further improvements in subject outcome that may become apparent with further analysis.

Given these data, it is expected that compounds of the invention would also work to treat patients with other types of coronaviruses.

The following examples are presented in order to more fully illustrate the preferred embodiments of the invention. They should in no way, however, be construed as limiting the broad scope of the invention.

### EXAMPLES

The Examples set forth below are for illustrative purposes only and are not intended to limit, in any way, the scope of the present invention.

### Materials and Methods:

In Vitro Tubulin Polymerization Assay. Bovine brain tubulin (0.4 mg, >97% pure) (Cytoskeleton, Denver, Colo.) was mixed with 10 μM of the test compounds and incubated in 100 μL of general tubulin buffer (80 mM PIPES, 2.0 mM MgCl₂, 0.5 mM EGTA, and 1 mM GTP) at pH 6.9. The absorbance of wavelength at 340 nm was monitored every 1 min for 20 min by the SYNERGY 4 Microplate Reader (Bio-Tek Instruments, Winooski, Vt.). The spectrophotometer was set at 37° C. for tubulin polymerization.

### Example 1

### Treatment of Subjects with COVID-19

Efficacy: Described in this example are the results of a clinical trial (COVID-19 study) that was a Phase 2, double-blind, placebo-controlled, proof-of-concept study of approximately 40 hospitalized patients with COVID-19 (SARS-CoV-2) at high risk for acute respiratory distress syndrome (ARDS). The primary endpoint of this study was the proportion of patients alive without respiratory failure at Day 29. Key secondary endpoints include the following: proportion of patients alive without respiratory failure at Day 15 and Day 22, all-cause mortality, days in intensive care unit (ICU), and days on mechanical ventilation. A summary of the efficacy observations in the intent to treat (ITT) population from this study are listed below. The p-values presented are from a chi-square analysis for responder analysis and t-test for continuous variables. Please note that no α was set in the Phase 2 study, however for small studies such as this, the α is generally set at 0.1. Therefore, any p-value <0.1 is considered statistically significant.

This protocol employed a responder analysis. A group of 39 subjects hospitalized for COVID-19 infection at high risk for acute respiratory distress syndrome (ARDS) were divided into two groups, a placebo group of 20 subjects and a treated group (group treated with compound 17ya) of 19 patients. The treated group was given a powder filled capsule containing 18 mg of compound 17ya taken by mouth daily until hospital discharge, up to a maximum of 21 days of dosing.

These hospitalized subjects were qualified as responders if they were alive without respiratory failure on Day 15, Day 22, and Day 29. A non-responder is a subject that EITHER died before the analysis day OR had respiratory failure on the analysis day. After a subject was discharged/deceased, to establish responder/non-responder status, a phone call was made to see if the subject was alive and had no evidence of respiratory failure on Day 15, Day 22, and Day 29 and in the safety follow-up of the study. For example, if a patient died on Day 8, they were a non-responder at Day 15, Day 22, and Day 29. If a patient had respiratory failure on Day 15, but not on Day 22 or Day 29, they would be a non-responder on Day 15, but not on Day 22 or Day 29. For this analysis, “all-cause mortality” was evaluated and anyone who died was taken as a non-responder. Responders also included subjects who were discharged from the hospital or have Grade 0-4 on the WHO Ordinal Scale for Clinical improvement on Day 15, Day 22, or Day 29 (evaluation day), and non-responders were subjects who died before the evaluation day or had Grade 5-8 on the WHO Ordinal Scale for Clinical Improvement on the evaluation day.

Primary endpoint: Compound 17ya reduced the proportion of patients that are non-responders, i.e., death or respiratory failure from 35.0% in the placebo group (7/20) to 15.8% (3/19) in the compound 17ya treated group at Day 15 (p=0.1697) and from 30.0% (6/20) in the placebo group to 10.5% (2/19) in the compound 17ya treated group at Day 29 (p=0.1322). See Table 2. This represents an approximately 55% reduction in treatment failures at Day 15 and a 65% reduction in treatment failures at Day 29 in the compound 17ya treated group compared to placebo.

TABLE 2
Proportion of subjects alive and free of respiratory
failure by visit (ITT population)

| | | 17ya | Placebo | Odds ratio/ |
|---|---|---|---|---|
| | Response | (n = 19) | (n = 20) | 95% CI/p-value |
| Day 15 | Responder | 16 (84.2%) | 13 (65.0%) | 2.56/(0.38, 17.23)/ |
| | Non-responder | 3 (15.8%) | 7 (35.0%) | 0.3342 |
| Day 22 | Responder | 16 (84.2%) | 14 (70.0%) | 2.14/(0.31, 14.88)/ |
| | Non-responder | 3 (15.8%) | 6 (30.0%) | 0.4433 |
| Day 29 | Responder | 17 (89.5%) | 14 (70.0%) | 2.69/(0.36, 20.39)/ |
| | Non-responder | 2 (10.5%) | 6 (30.0%) | 0.3379 |

Compound 17ya reduced the proportion of patients who died up to 60 days after initiation of treatment from 30% (6/20) in the placebo group to 5% (1/19) in the compound 17ya treated group. This is an approximately 82% reduction in mortality in the compound 17ya treated group.

Compound 17ya reduced the days on mechanical ventilation from an average of 5.4 days in the placebo group to 1.6 days in the compound 17ya treated group. This represents a 3.4-fold increase in the days on mechanical ventilation in the placebo group compared to the compound 17ya treated group. See Table 3.

Compound 17ya reduced the days in ICU from an average of 9.6 days in the placebo group to 3.0 days in the compound 17ya treated group. This represents a 3.2-fold increase in the days in the ICU in the placebo group compared to the compound 17ya treated group. See Table 3.

TABLE 3
Days on Mechanical Ventilation

| Treatment | N | mean | SD | P-value |
|---|---|---|---|---|
| Compound 17ya | 19 | 1.6 | 6.64 | 0.4836 |
| Placebo | 20 | 5.4 | 10.16 | |

Days in ICU

| | | | | |
|---|---|---|---|---|
| Compound 17ya | 19 | 3.0 | 7.16 | 0.0742 |
| Placebo | 20 | 9.6 | 11.54 | |

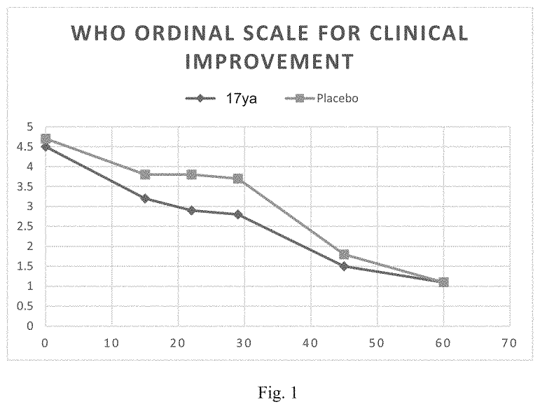

FIG. 1 illustrates the mean WHO Ordinal Scale for Clinical Improvement by Day (0=baseline). The area under the mean curve is 153 in the group treated with compound 17ya and 182 in the Placebo group, indicating greater morbidity in the placebo population and suggesting a clinical improvement associated with receiving compound 17ya.

As the study was limited in sample size based on FDA comments received during the IND review process, the study sponsor (Veru, Inc.) has conducted post-hoc, sub-group analyses of the data from the study. The following additional observations are made from this study:

In the compound 17ya treated group there was one patient who was noncompliant with oxygen supplementation. This patient was noncompliant with standard of care in this study. An analysis of the primary endpoint excluding this patient (MITT population) from the analysis shows a 30% failure rate in the Placebo group (same as Table 2) compared to a 5.6% failure rate in the compound 17ya treated group at Day 29 (lower than in Table 2). This represents an 81% reduction in treatment failures.

It is well recognized that older patients are at higher risk for death and respiratory failure in patients with COVID-19 compared to younger patients. In an analysis of treatment failures in patients >60 years of age showed that a statistically significant and clinically meaningful reduction in treatment failures were observed in the compound 17ya treated group compared to placebo in this high-risk population.

| | | Treatment failures | |
|---|---|---|---|
| | N | at Day 29 | p-value |
| Compound | 11 | 1 (9%) | 0.0456 |
| 17ya | | | (chi-square) |
| Placebo | 8 | 4 (50%) | |

A risk factor for an adverse clinical outcome in a patient with COVID-19 is the severity of disease at presentation. To assess this risk factor, an analysis of patients with a WHO Score of Disease Severity ≥5 at baseline was performed. The outcome of this analysis shows a statistically significant and clinically meaningful reduction in treatment failures were observed in the compound 17ya treated group compared to placebo in this high-risk population. Also, clinically meaningful reduction (78%; not shown) in mortality was observed in the compound 17ya treated (1/10 or 10%) group compared to placebo (6/13 or 46%) in this high risk population.

| | | Treatment failures | |
|---|---|---|---|
| | N | at Day 29 | p-value |
| Compound | 9* | 1 (11%) | 0.0827 |
| 17ya | | | (chi-square) |
| Placebo | 13 | 6 (46%) | |

*one patient in the compound 17ya treated group was noncompliant with oxygen therapy and is excluded from this modified intent to treat (MITT) analysis.

An analysis of the days in ICU in evaluable patients showed a statistically significant and clinically meaningful reduction in days in ICU in the compound 17ya treated group compared to placebo.

| | | Mean days in ICU | |
|---|---|---|---|
| | N | (± st.dev) | p-value |
| Compound | 18 | 3.00 ± 7.37 | 0.0469 |
| 17ya | | | (t-test) |
| Placebo | 20 | 9.55 ± 12.56 | |

Additionally, the proportion of patients that were in the ICU for ≥3 days on study is statistically significantly higher in the placebo group compared to the compound 17ya treated group.

| | | Treatment failures | |
|---|---|---|---|
| | N | at Day 29 | p-value |
| Compound | 18 | 4 (22%) | 0.0390 |
| 17ya | | | (chi-square) |
| Placebo | 20 | 11 (55%) | |

In this study, patients were permitted to receive standard of care. At the time of the study, the standard of care included treatment with remdesivir and/or dexamethasone under an Emergency Use Authorization. There were eleven patients in the study that did not receive either remdesivir or dexamethasone (6 in the compound 17ya treated group and 5 in the placebo group). An analysis of patients that received the recognized standard of care was conducted. Specifically, the days in ICU and the days on mechanical ventilation were compared between the treatment groups. In this population, in patients that received standard of care, no patient treated with compound 17ya required admission in the ICU or mechanical ventilation and there were no mortalities in this patient group. In the placebo group, 53% (8/16) required ICU admission with an average of 9.5 days in the ICU, 20% (3/15) required mechanical ventilation with an average of 3.9 days of mechanical ventilation, and 27% (4/15) died on study.

Overall, the study sponsor proposes that compound 17ya shows strong clinically meaningful outcomes in this small, proof-of-concept, Phase 2 study with statistically significant observations in reductions in death in the ITT population and in post-hoc, high-risk sub-group analyses, and days in ICU. It is important to note that all the parameters measured in the study show clinically meaningful outcomes with compound 17ya compared to placebo and there are no parameters that do not indicate benefit with compound 17ya treatment compared to placebo although some parameters do not reach statistical significance in this small study.

Safety: The overall safety conclusions are: (1) There were no treatment related serious adverse events observed on the study; (2) There were no treatment related adverse events observed on the study; and (3) The treatment emergent adverse events that were observed in at least 2 patients in either treatment group in the study are presented in Table 4. There is no imbalance against compound 17ya in adverse events observed in the study.

TABLE 4
COVID-19 Study: Treatment Emergent Adverse Events Observed
in ≥2 Patients in Either Treatment Group by Preferred Term

| | Compound | |
|---|---|---|
| | 17ya 18 mg | Placebo |
| | (n = 19) | (n = 20) |
| Preferred Term | N (%)/events | N (%)/events |
| Any | 10 (52.6)/27 | 11 (55.0)/41 |
| Constipation | 2 (10.5)/2 | 2 (10.0)/2 |
| Septic shock | 1 (5.3)/1 | 2 (10.0)/2 |
| Alanine aminotransferase | 1 (5.3)/1 | 2 (10.0)/2 |
| increased | | |
| Aspartate aminotransferase | 2 (10.5)/2 | 1 (5.0)/1 |
| increased | | |
| Acute kidney injury | 0 | 2 (10.0)/2 |
| Pneumomediastinum | 0 | 2 (10.0)/2 |
| Pneumothorax | 1 (5.3)/1 | 3 (15.0)/3 |
| Respiratory failure | 0 | 4 (20.0)/4 |

The treatment emergent serious adverse events observed in the study are presented in Table 5. There is no imbalance against compound 17ya in serious adverse events observed in the study.

TABLE 5
COVID-19 Study: Serious Adverse Events Observed
by System Organ Class and Preferred

| | Compound | |
|---|---|---|
| | 17ya 18 mg | Placebo |
| System Organ Class | (n = 19) | (n = 20) |
| Preferred Term | N (%)/events | N (%)/events |
| Any | 3 (15.8)/3 | 4 (20.0)/4 |
| Cardiac disorders | 1 (5.3)/1 | 0 |
| Cardiac arrest | 1 (5.3)/1 | 0 |
| Infections and infestations | 1 (5.3)/1 | 2 (10.0)/2 |
| COVID-19 | 0 | 1 (5.0)/1 |
| Septic shock | 1 (5.3)/1 | 1 (5.0)/1 |
| Nervous system disorders | 0 | 1 (5.0)/1 |
| Encephalopathy | 0 | 1 (5.0)/1 |
| Renal and urinary disorders | 0 | 1 (5.0)/1 |
| Acute kidney injury | 0 | 1 (5.0)/1 |
| Respiratory, thoracic and | 1 (5.3)/1 | 2 (10.0)/2 |
| mediastinal disorders | | |
| Epistaxis | 1 (5.3)/1 | 0 |
| Respiratory failure | 0 | 2 (10.0)/2 |

Overall, compound 17ya was well tolerated in this patient population with no clinically relevant safety observations in the compound 17ya treated group.

### Example 2

### Treatment of Subjects with COVID-19 Phase 3 Study

The Phase 3 clinical trial was a double-blind placebo controlled study with a target enrollment of 210 patients randomized in a 2:1 ratio to Compound 17ya 9 mg capsules (bioequivalent to 18 mg in the phase 2 study of Example 1) versus identically appearing placebo capsules. The target patient population with moderate to severe COVID-19 (SARS-CoV-2) were hospitalized patients on oxygen supplementation at high risk for acute respiratory distress syndrome (ARDS) and death. Randomization into the study was stratified by WHO ordinal scale score. Patients with WHO scores of 4 (oxygen by mask or nasal prongs), patients that have a documented comorbidity such as, asthma, chronic lung disease, diabetes, hypertension, severe obesity (BMI ≥40), 65 years of age or older, primarily reside in a nursing home or long-term care facility, or immunocompromised. WHO 5 (non-invasive ventilation or high-flow oxygen) or WHO 6 (intubation and mechanical ventilation and have a blood oxygen level ≤94% on room air at screening. The different WHO score patients were equally distributed between the treatment groups. The key exclusion criteria for the study were pregnancy or currently breast feeding, WHO 7 (required ventilation plus additional organ support—long term pressors, renal replacement therapy, or extracorporeal membrane oxygen), alanine aminotransferase or aspartate aminotransferase >3 times the upper limit of normal, total bilirubin >upper limit of normal, creatinine clearance <60 mL/min, moderate to severe renal impairment, and hepatic impairment. Patients in both treatment groups were allowed to receive standard of care including remdesivir, dexamethasone, anti-IL6 receptor antibodies, and JAK inhibitors. A planned interim analysis was conducted in the first 150 patients randomized into the study. The trial was conducted in the United States, Brazil, Bulgaria, Colombia, Argentina and Mexico. COVID-19 infections treated in the study included several variants including Delta, and Omicron.

Procedure: Patients were randomized to Compound 17ya treated group versus the placebo group in a 2:1 ratio. Dosing with study Compound 17ya was for up to 21 days of daily dosing. If a patient was discharged from the hospital prior to Day 21, then dosing was discontinued upon discharge from the hospital.

Efficacy: The primary endpoint of the study was to assess the effect of Compound 17ya on all-cause mortality (proportion of patients who died up to Day 60 on study) compared to placebo in the intent-to-treat (ITT) population. The key secondary endpoints in the study were to compare days in the intensive care unit, days on mechanical ventilation, proportion of patients with respiratory failure or death on study, days in the hospital, and change from baseline in viral load. The multivariate analysis of the primary endpoint included treatment, geographical region, WHO ordinal scale score at baseline, standard of care use, and gender. Prespecified subgroup analysis of the primary endpoint included by region (country), by WHO ordinal scale score at baseline, and by standard of care use during the study (dexamethasone and remdesivir).

Safety: Safety assessment included treatment-emergent adverse events, serious adverse events and adverse events leading to discontinuation from the study starting at randomization into the study through day 60 of the study. Medical Dictionary for Regulatory Activities (MedRA), version 24.0 was used for safety coding. The incidence of events was provided for each treatment group and included all patients randomized into the study.

Statistical Analysis:

The study was designed with a 204 patient sample size (134 Compound 17ya and 70 placebo) with a two-sided alpha of 0.05 and 92.8% power to detect an approximately 50% relative reduction in deaths in the Compound 17ya treated group compared to placebo (expected mortality rate in the placebo group=30%).

The study was designed to have a planned interim analysis of the first 150 patients randomized into the study. The alpha spend at the interim analysis is 0.0160 with the final analysis alpha remaining of 0.0452. The study had an independent data monitoring committee (IDMC) made up of three qualified medical doctors and a non-voting statistician who met every 4-6 weeks during the course of the study to review unblinded safety data with the option to discontinue the study for safety reasons if the data warranted. The IDMC recommended to continue the study as planned upon review of the safety data at each IDMC meeting. At the time of the interim analysis, the IDMC was chartered to review unblinded efficacy data in the form of the primary endpoint, proportion of patients who died on study up to Day 60. At that timepoint, the IDMC members had the option to vote to discontinue the study for reasons of demonstrated efficacy. The criteria was that the two-sided p-value around the primary endpoint should be less than 0.0160.

Patients

The interim analysis population included the first 150 patients randomized into the study (comprising 74% of all randomized subjects), 98 patients were randomized into the Compound 17ya treatment group and 52 patients were randomized into the placebo group. The p-value in the interim analysis was 0.0029 with a 55.2% relative reduction in mortality in the patients treated with Compound 17ya 9 mg compared to placebo and 24.9% absolute reduction. The baseline demographic and clinical characteristics were similar in the two groups in the interim analysis. The patients enrolled had moderate to severe COVID-19 infections with a mean oxygen saturation (SpO₂) on room air at baseline of 92.5% and required supplemental oxygen. The proportion of patients requiring oxygen by mask or nasal prongs, non-invasive ventilation or high-flow oxygen, or intubation and mechanical ventilation were similar between the treatment groups. The distribution of common risk factors for ARDS and death were similar between the treatment groups included hypertension (59.2% Compound 17ya, 61.5% placebo) age ≥65 years (45.9% Compound 17ya, 50% placebo), diabetes (35.7% Compound 17ya, 40.4% placebo), and obesity as defined as BMI ≥35 (34.7% Compound 17ya, 27.5% placebo). COVID-19 unvaccination rates were also similar (70.4% Compound 17ya, 75% placebo). Standard of care were also similar between the two groups where dexamethasone and remdesivir were the most common treatments (dexamethasone: 83.7% for Compound 17ya, 80.7% placebo and remdesivir: 354.7% Compound 17ya, 28.8% placebo). Table 6 summarizes the data.

TABLE 6
Demographic and Clinical Characteristics of the Patients
(Interim Analysis Population)

| | Compound | | Overall |
|---|---|---|---|
| | 17ya | Placebo | (N = |
| Parameter | 9 mg (N = 98) | (N = 52) | 150) |
| Age | | | |
| Mean (SD) | 59.4 (14.57) | 60.3 (15.02) | 59.7 |
| | | | (14.68) |
| Median | 64.0 | 64.0 | 64.0 |
| Min, Max | 25, 92 | 19, 86 | 19, 92 |
| Age, Category, n (%) | | | |
| <65 years | 53 (54.1) | 26 (50.0) | 79 (52.7) |
| ≥65 years | 45 (45.9) | 36 (50.0) | 71 (47.3) |
| Sex, n (%) | | | |
| Male | 69 (70.4) | 33 (63.5) | 102 |
| | | | (68.0) |
| Female | 29 (29.6) | 19 (36.5) | 48 (32.0) |
| BMI | | | |
| Mean (SD) | 33.0 (7.39) | 32.4 (7.96) | 32.8 |
| | | | (7.57) |
| Median | 32.0 | 31.1 | 31.65 |
| Min, Max | (20.2, 61.7) | (22.9, 69.4) | (20.2, |
| | | | 69.4) |
| Race, n (%) | | | |
| White | 83 (84.7) | 46 (88.5) | 129 |
| | | | (86.0) |
| Black or African American | 6 (6.1) | 2 (3.8) | 8 (5.3) |
| Asian | 2 (2.0) | 0 | 2 (1.3) |
| American Indian or Alaska | 1 (1.0) | 2 (3.8) | 3 (2.0) |
| Native | | | |
| Other | 6 (6.1) | 2 (3.8) | 9 (5.3) |
| WHO 9-point Ordinal Scale | | | |
| for Clinical Improvement | | | |
| (descriptive) | | | |
| Mean (SD) | 4.8 (0.61) | 4.8 (0.65) | 4.8 (0.62) |
| Median | 5.0 | 5.0 | 5.0 |
| Min, Max | 4, 6 | 4, 6 | 4, 6 |
| WHO 9-point Ordinal Scale | | | |
| for Clinical Improvement | | | |
| (frequencies), n (%) | | | |
| WHO 4-Oxygen by mask | 33 (33.7) | 18 (34.6) | 51 (34.0) |
| or nasal prong | 56 (57.1) | 28 (53.8) | 84 (56.0) |
| WHO 5-Non-invasive | | | |
| ventilation or high-flow | | | |
| oxygen | | | |
| WHO 6-intubation and | 9 (9.2) | 6 (11.5) | 15 (10.0) |
| mechanical ventilation | | | |
| Comorbidities | | | |
| Cancer, n (%) | 7 (7.1) | 0 (0.0) | 7 (4.7) |
| Diabetes, n (%) | 35 (35.7) | 21 (40.4) | 56 (37.3) |
| Hypertension, n (%) | 58 (59.2) | 32 (61.5) | 90 (60.0) |
| History of heart failure , n | 4 (4.1) | 3 (5.8) | 7 (4.7) |
| (%) | | | |
| Pneumonia, n (%) | 46 (46.9) | 29 (55.8) | 85 (56.7) |
| Renal issues, n (%) | 12 (12.2) | 4 (7.7) | 16 (10.7) |
| Respiratory issues, n (%) | 65 (66.3) | 28 (53.8) | 93 (62.) |
| Asthma, n (%) | 11 (1.0) | 3 (5.8) | 14 (9.3) |
| Oxygen Saturation (SpO₂) | | | |
| at baseline, % | | | |
| N | 98 | 52 | 150 |
| Mean (SD) | 92.7 (3.43) | 92.0 (7.51) | 92.5 |
| | | | (5.20) |
| Median | 93.0 | 94.0 | 93.0 |
| Min, Max | 84, 100 | 48, 100 | 48, 100 |
| Time from Admission to | | | |
| Randomization (days) | | | |
| N | 98 | 52 | 150 |
| Mean (SD) | 3.4 (2.22) | 4.0 (2.79) | 3.6 |
| | | | (2.45) |
| Median | 3.0 | 3.5 | 3.0 |
| Min, Max | 0, 13 | 0, 11 | 0, 13 |
| Vaccination Status, n (%) | | | |
| Not Vaccinated | 69 (70.4) | 39 (75.0) | 108 |
| | | | (72.0) |
| Vaccinated (1, 2, or | 29 (29.6) | 13 (25.0) | 42 (28.0) |
| 3 shots) | | | |
| Standard of care, n (%) | | | |
| Dexamethasone (yes) | 82 (83.7) | 42 (80.7) | 114 |
| | | | (76.0) |
| Remdesivir (yes) | 34 (34.7) | 15 (28.8) | 49 (32.7) |
| Tocilizumab (yes) | 10 (10.2) | 5 (9.6) | 15 (10.0) |
| Baricitinib or Tofacitinib | 10 (10.2) | 8 (15.4) | 18 (12.0) |
| (yes) | | | |

Treatment with Compound 17ya resulted in a mortality rate of 20% versus 45% for those patients treated with placebo. Among the secondary endpoints, there was a 49% relative reduction in days on mechanical ventilator (p=0.0016) and 26% reduction in days in hospital (p=0.0277) as compared to placebo treatment.

In the primary efficacy endpoint of mortality up to Day 60, a clinically meaningful and statistically significant 24.9% absolute reduction and 55.2% relative reduction in mortality was observed in the group treated with Compound 17ya compared to the placebo treated group (odds ratio=3.20, 95% CI, 1.44, 7.09; p=0.0043). Table 7 summarizes this data.

TABLE 7
Efficacy Endpoints

| | Compound | Placebo | | |
|---|---|---|---|---|
| | 17ya | | | |
| Result | (9 mg) (N = 98) | (N = 52) | | |
| Alive | 75 (79.8) | 28 (54.9) | | |
| Dead | 19 (20.2) | 23 (45.1) | | |
| Vital status | 4 | 1 | | |
| missing at | | | | |
| Day 60 | | | | |
| Treatment | | | | |
| Comparison | Odds Ratio | 95% CI | p-value | |
| Compound 17ya | 3.20 | (1.44, 7.09) | 0.0043 | |
| 9 mv vs Placebo | | | | |
| | Compound | | | |
| | 17ya | | Relative | |
| Days on Study | 9 mg | Placebo | Difference | p-value |
| Day 15 | 7/94 (7.4%) | 13/51 (25.5%) | −71.0% | 0.0046¹ |
| Day 29 | 15/94 (16.0%) | 18/51 (35.3%) | −54.7% | 0.0122¹ |
| Day 60 | 19/94 (20.2%) | 23/51 (45.1%) | −55.2% | 0.0043² |

¹The p-values were generated using a Fisher's Exact Test.
²Primary endpoint of the study. P-value generated using the logistic regression with the multivariate analysis.

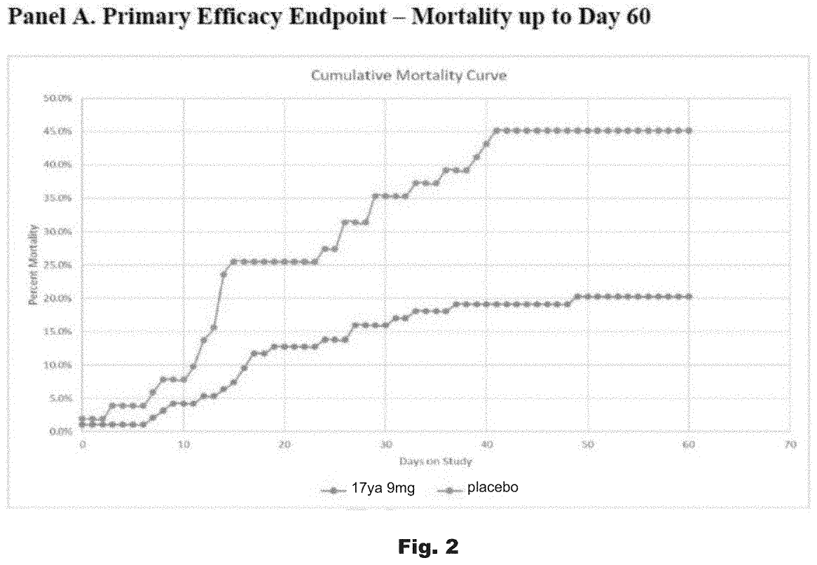

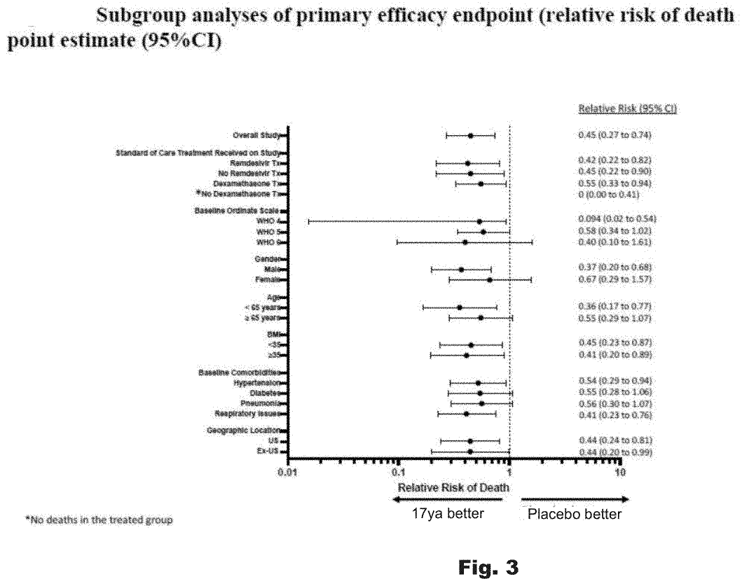

As seen in FIG. 2 (cumulative percent mortality over time (days of study)), the beneficial effects of treatment with Compound 17ya was observed starting as early as Day 3 after dosing and by Day 15, clinically meaningful and statistically significant reduction in mortality were observed. These effects were maintained through Day 29 (a standard endpoint that other studies have used) with a placebo mortality rate of 35.3% compared to 16.0% mortality rate for those treated with Compound 17ya, and absolute reduction of 19.3% and relative reduction of 54.7% (=0.0112). From Day 29 to Day 60, the death rated increased by 9.8% in the placebo treated group and by 4.2% in the Compound 17ya treated group (absolute change). FIG. 3 illustrates the results of a subgroup analyses evaluating the relative risk of death in patients treated that were consistent with the overall study results favoring treating with Compound 17ya regardless of treatment received, baseline WHO ordinate clinical score, sex, age, baseline comorbidities, BMI, or geographic location.

A sensitivity assessment was conducted for the primary efficacy endpoint of all-cause mortality for the full final data set (ITT population) of 204 randomized patients which had similar results as the interim efficacy analysis with Compound 17ya treatment resulting in a 50.9% reduction in deaths compared to the placebo treated group (p=0.0037, Fishers Exact Test).

The treatment with Compound 17ya when compared to treatment with placebo results in a statistically significant reduction in key secondary endpoints. There was a 44% reduction in days in the ICU (LS mean of −13.4 days 95% CI −21.5, −5.3; p=0.0013); 49% reduction in days on mechanical ventilation (LS mean of −13.9 days 95% CI −22.4, −5.4; p=0.0016); and 26% reduction in days in the hospital (LS mean −8.4 days 95% CI −15.8, −0.9; p=0.0277). Table 8 summarizes the data.

TABLE 8
Secondar Efficacy Endpoints
Days in ICU

| | Mean | | Median | Min, |
|---|---|---|---|---|
| Treatment | (days) | SD | (days) | Max |
| Compound 17ya 9 mg (n = 98) | 17.4 | 23.83 | 4.0 | 0, 60 |
| Placebo (n = 52) | 30.8 | 27.80 | 17.0 | 0, 60 |
| Treatment Comparison | LS mean | SE | 95% CI | p-value |
| Compound 17ya vs Placebo | −13.5 | 4.08 | (−21.6, | 0.0013 |
| | | | −5.4) | |

Days on Mechanical Ventilation

| | Mean | | Median | Min, |
|---|---|---|---|---|
| Treatment | (days) | SD | (days) | Max |
| Compound 17ya 9 mg | 14.3 | 24.05 | 0.0 | 0, 60 |
| (n = 98) | | | | |
| Placebo (n = 52) | 28.1 | 29.54 | 4.0 | 0, 60 |
| Treatment Comparison | LS mean | SE | 95% CI | p-value |
| Compound 17ya vs Placebo | −13.9 | 4.31 | (−22.4, | 0.0016 |
| | | | −5.4) | |

Days in Hospital

| | Mean | | Median | Min, |
|---|---|---|---|---|
| Treatment | (days) | SD | (days) | Max |
| Compound 17ya 9 mg | 25.6 | 22.87 | 14.0 | 0, 60 |
| (n = 98) | | | | |
| Placebo (n = 52) | 34.6 | 24.63 | 30.5 | 0, 60 |
| Treatment Comparison | LS mean | SE | 95% CI | p-value |
| Compound 17ya vs Placebo | −8.4 | 3.76 | (−15.8, | 0.0277 |
| | | | −0.9) | |

The adverse events and serious adverse events observed in the study were consistent with patients that have a serious COVID-19 illness. The proportion of patients that experienced any adverse event was lower in the Compound 17ya treated group (61.5%) compared to the placebo treated group (78.3%). The most frequently reported adverse events in either group were respiratory failure (Compound 17ya 9.2% vs. placebo 17.4%); acute kidney injury (Compound 17ya 8.5% vs. placebo 11.6%); pneumothorax (Compound 17ya 0.8% vs placebo 10.1%); pneumonia bacterial (Compound 17ya 0% vs placebo 7.2%); and hypotension (Compound 17ya 2.3% vs. placebo 11.6%). The proportion of patients with a serious adverse event observed was lower for those treated with Compound 17ya (29.2%) compared to those treated with placebo (46.4%). The most frequently reported serious adverse events in either group were respiratory failure (Compound 17ya 9.2% vs. placebo 17.4%); acute kidney injury (Compound 57ya 3.8% vs. placebo 8.7%); pneumothorax (Compound 17ya 0.8% vs. placebo 8.7%); septic shock (Compound 17ya 1.5% vs. placebo 7.2%); and acute respiratory failure (Compound 17ya 5.4% vs. placebo 58%). Adverse events leading to discontinuation were 4.7% for those treated with Compound 17ya vs 5.9% for those treated with placebo. Table 9 summarizes this data.

TABLE 9
Summary of Adverse Events and Serious Adverse
Events for ITT population

| | |
|---|---|
| Compound 17ya | Placebo |
| (N = 130) | (N = 69) |
| N(%)/Events | N(%)/Events |

Treatment-Emergent Adverse Events
(≥5% of patients in either treatment group

| | | |
|---|---|---|
| Any | 80 (61.5)/328 | 54 (78.3)/279 |
| Atrial fibrillation | 5 (3.8)/5 | 4 (5.8)/4 |
| Bradycardia | 5 (3.8)/6 | 5 (7.2)/5 |
| Constipation | 8 (6.2)/8 | 6 (8.7)/10 |
| Pneumonia | 7 (5.4)/11 | 8 (11.6)/11 |
| Pneumonia bacterial | 0 | 5 (7.2)/5 |
| Septic Shock | 2 (1.5)/2 | 5 (7.2)/5 |
| Urinary tract infection | 8 (6.2)/8 | 1 (1.4)/1 |
| Hyperkalemia | 5 (3.8)/5 | 6 (8.7)/7 |
| Hypernatremia | 6 (4.6)/6 | 4 (5.8)/4 |
| Hypokalemia | 5 (3.8)/6 | 4 (5.8)/4 |
| Hypophosphatemia | 2 (1.5)/3 | 4 (5.8)/5 |
| Anxiety | 3 (2.3)/4 | 4 (5.8)/4 |
| Acute kidney injury | 11 (8.5)/11 | 8 (11.6)/8 |
| Acute respiratory failure | 9 (6.9)/9 | 4 (5.8)/5 |
| Bronchitis chronic | 2 (1.5)/2 | 4 (5.8)/4 |
| Hypoxia | 3 (2.3)/4 | 4 (5.8)/4 |
| Pneumothorax | 1 (0.8) /1 | 7 (10.1)/7 |
| Respiratory failure | 12 (9.2)/13 | 12 (17.4)/12 |
| Hypotension | 3 (2.3)/3 | 8 (11.6)/8 |

Treatment Emergent Serious Adverse Events
(>2% of the patients in either treatment group)

| | | |
|---|---|---|
| Any | 38 (29.2)/82 | 32 (46.4)/84 |
| Cardiac arrest | 0 | 3 (4.3)/4 |
| COVID-19 | 2 (1.5)/2 | 2 (2.9)/2 |
| Pneumonia | 3 (2.3)/5 | 4 (5.8)/5 |
| Pneumonia bacterial | 0 | 2 (2.9)/2 |
| Sepsis | 3 (2.3)/4 | 2 (2.9)/2 |
| Septic Shock | 2 (1.5)/2 | 5 (7.2)/5 |
| Acute kidney injury | 5 (3.8)/5 | 6 (8.7)/6 |
| Acute respiratory failure | 7 (5.4)/7 | 4 (5.8)/5 |
| Hypoxia | 2 (1.5)/3 | 3 (4.3)/3 |
| Pneumothorax | 1 (0.8)/1 | 6 (8.7)/6 |
| Pulmonary embolism | 3 (2.3)/3 | 3 (4.3)/3 |
| Respiratory failure | 12 (9.2)/13 | 12 (17.4)/12 |

The secondary efficacy endpoints showed that those treated with Compound 17ya resulted in a significant reduction compared to those treated with placebo in day in the ICU, days on mechanical ventilation, and days in the hospital. Significantly fewer serious adverse events and adverse events were reported for those treated with Compound 17ya as compared to those treated with placebo, as well as, fewer treatment discontinuations due to adverse events. In particular, patients treated with Compound 17ya had fever COVID-19 related morbidities especially respiratory failure, acute renal injury, cardiac arrest, septic shock, and/or hypotension when compared to those treated with placebo.

After this review and consideration of the safety comparison between the treatment groups, the IDMC members voted unanimously to discontinue the Phase 3 clinical trial for reasons of demonstrated efficacy. The IDMC members found no safety issues of clinical note in the Compound 17ya treated group compared to placebo.

The Compound 17ya results indicate that the pharmacological activity of Compound 17ya is independent of COVID-19 variant type and that its dual anti-viral and anti-inflammatory properties and Phase 3 efficacy and safety results, can yield a much needed oral therapy for hospitalized moderate to severe COVID-19 patients.

Discussion: Compound 17ya is a novel microtubule disruptor that has dual antiviral and anti-inflammatory activities. Compound 17ya 9 mg oral daily dosing (which is a different formulation that is bioequivalent to 18 mg of the formulation in the phase 2 study) up to 21 days demonstrated significant efficacy in a randomized, double blind, placebo-controlled global Phase 3 clinical trial in hospitalized adult patients with moderate to severe COVID-19 who were at high risk for ARDS and death. Based on protocol prespecified criteria for efficacy and safety interim analysis results, the independent data monitoring committee (IDMC) indicated that the endpoints have been met and recommended stopping the study early as results indicated that compound 17ya demonstrated a clinically meaningful and statistically significant 24.9% absolute and 55.2% relative reduction in all-cause mortality by Day 60, the primary efficacy endpoint of the study. The cumulative mortality analysis showed that the reduction in deaths with compound 17ya occurred within the first week of treatment and reached a significant 54.7% relative reduction in deaths at Day 29. This efficacy was further supported by the consistency of the subgroup analyses of the primary endpoint; a reduction in death with compound 17ya treatment compared to placebo regardless of standard of treatment received, baseline WHO ordinate score, sex, age, baseline comorbidities, BMI, or geographic location. Furthermore, the secondary efficacy endpoints showed compound 17ya treatment resulted in a significant reduction compared to placebo in days in the ICU, days on mechanical ventilation, and days in the hospital. Compound 17ya was well tolerated and safe. Significantly fewer serious adverse event and adverse events were reported for compound 17ya compared to placebo. There were also fewer treatment discontinuations due to adverse events in compound 17ya group compared to placebo. The Phase 3 reported safety profiles suggest that compound 17ya treatment may have resulted in fewer COVID-19 related morbidities especially respiratory failure, acute renal injury, cardiac arrest, septic shock, and hypotension.

Vaccinations remain the mainstay for prevention of serious COVID-19 infections and death. Most patients will recover from an acute COVID-19 illness. New antivirals, molnuparivir and nirmatrelvir, when taken in a prehospital setting within 3-5 days after COVID-19 symptom onset, reduce the incidence of COVID-19 related hospitalization or death. For those patients who do progress to moderate to severe COVID-19 illness requiring hospitalization, the risk for death remains high and remains an unmet clinical need. In this setting, however, the antiviral, molnupravir, did not demonstrate clinical benefit. Although reported COVID-19 mortality rates in hospitalized patients are highly variable depending on the severity of the COVID infection, the presence and number of high-risk comorbidities, country and accuracy of reporting; recent mortality rates have been reported for high-risk COVID-19 patients in the 21.4% to 70.5% range.

In the current Phase 3 COVID-19 study, compound 17ya was evaluated in hospitalized patients with moderate to severe COVID-19 illness. The inclusion criteria were selected by design to enrich the study for patients who were at the highest risk for ARDS and death by COVID-19. Enrolled hospitalized patients had to demonstrate moderate to severe COVID-19 illness (at least oxygen supplementation with SpO₂ ≤94% on room air); for patients with WHO score of 4 (receiving supplemental oxygen), they had to have a comorbidity that placed them at high risk for death; and no limitation was placed on the duration of COVID-19 symptoms prior to enrollment. Accordingly, the actual mortality rate in the placebo group for this study, which was expected to reflect the sickest COVID-19 hospitalized patients receiving standard of care, was 35.3% at Day 29, consistent with previously reported death rates, and by Day 60, the death rate in the placebo group further increased to 45.1%. This sobering death rate, with the COVID-19 pandemic in its 3^{rd} year, underscores the need for new, effective treatments in hospitalized patients with moderate to severe COVID-19 at risk for ARDS and death.

It is apparent that the mortality rate for hospitalized moderate to severe COVID-19 patients with available therapies, the antiviral, remdesivir, or immunomodulators/anti-inflammatory agents, remains high. By targeting microtubule trafficking, compound 17ya has both dual anti-inflammatory and antiviral activities. The Phase 3 clinical data shows compound 17ya treatment demonstrated a clinically meaningful and significant reduction in mortality in hospitalized patients with moderate to severe COVID-19 who are at high risk for ARDS and death.

### Example 3

### Treatment of Subjects with SARS-CoV-2 Infection in Hospitalized Patients with Moderate to Severe COVID-19 Infection Who are at High Risk for Acute Respiratory Distress Syndrome (ARDS)

Importantly, acute respiratory distress syndrome (ARDS) remains a frequent complication of severe COVID-19 infection. Up to 33% of hospitalized patients with COVID-19 have ARDS and 75% to 92% of patients admitted to the intensive care unit (ICU) with COVID-19 have ARDS. The mortality rate of COVID-19 associated ARDS is 45% and there is a 90% incidence of ARDS among patients who died from COVID-19.

Patients at high risk for ARDS are defined as patients who: (1) are on supplemental oxygen with at least one known comorbidity that has been identified as a risk factor for ARDS, including asthma, chronic lung disease, diabetes, hypertension, severe obesity (BMI ≥40), 65 years of age or older, primarily reside in a nursing home or long-term care facility, or immunocompromised; or (2) are on non-invasive ventilation or high-flow oxygen; or (3) are intubated and placed on mechanical ventilation; and have an oxygen saturation (SpO₂) level ≤94% on room air prior to receiving oxygen support.

Overview of Efficacy

Evidence of effectiveness was derived from a Phase 2 study (Study A) and a Phase 3 study (Study B) investigating the safety and efficacy of Compound 17ya for the treatment of hospitalized patients with moderate to severe COVID-19 infection who are at high risk for ARDS. It is important to note that Study B was stopped early by an Independent Data Monitoring Committee due to clear clinical benefit and no safety concerns identified. Further, results from the Full Study population of 204 patients from this study demonstrated that Compound 17ya treatment resulted in a 51.6% relative reduction in deaths compared to the placebo group (p=0.0046). These results in the Full Study population confirm the Interim Analysis conclusions of clinically meaningful and statistically significant efficacy profile and a favorable safety profile which led to the Phase 3 clinical study stopping early.

Design of Study A

The study was entitled “Randomized, Placebo-Controlled, Phase 2 Study of VERU-111 for the Treatment of Severe Acute Respiratory Syndrome Coronavirus 2 (SARS-CoV-2) In Patients at High Risk for Acute Respiratory Distress Syndrome (ARDS).” This study was a multicenter, randomized, double-blind, placebo-controlled clinical study to determine efficacy and safety of 18 mg PIC Compound 17ya for the treatment of hospitalized moderate to severe COVID-19 adult patients who are at high risk for ARDS. A placebo control was chosen to establish equipoise in this small study, and patients were randomized to Compound 17ya treatment or placebo in a 1:1 manner. The study was conducted entirely in the United States between 18 Jun. 2020 and 9 Dec. 2020.

Key inclusion criteria for this study were the following: subjects ≥18 years of age with documented evidence of COVID-19 infection (by standard diagnostic method). Subjects with high risk for developing ARDS due to a known comorbidity for being at risk such as: asthma (moderate to severe); Chronic Lung Disease, Diabetes; Chronic Kidney Disease being treated with dialysis; Severe Obesity (BMI ≥40); 65 years of age or older; or primarily reside in a nursing home or long-term care facility, or immunocompromised. Other key inclusion criteria include Peripheral capillary oxygen saturation (SpO2) ≤94% on room air at screening.

Key exclusion criteria were the following: (1) pregnant or breastfeeding; (2) required ventilation+additional organ support—pressors, renal replacement therapy (RRT), ECMO (WHO Ordinal Scale for Clinical Improvement—Score of 7) and subjects who required ventilation with a WHO Ordinal Scale for Clinical Improvement—Score of 6 for >5 days at screening; (3) moderate to severe renal impairment; or (3) hepatic impairment.

Standard of care treatments available for hospitalized patients with COVID-19 under Emergency Use Authorization by the US FDA were allowed in the study. Subjects received either 18 mg PIC of Compound 17ya or matching placebo, orally or via nasogastric tube, daily for up to 21 days or until the subject was discharged from the hospital, whichever came first.

Design of Study B

Study B was entitled “Phase 3, Randomized, Placebo-Controlled, Efficacy and Safety Study of VERU-111 for the Treatment of Severe Acute Respiratory Syndrome Coronavirus 2 (SARS-CoV-2) in Patients at High Risk for Acute Respiratory Distress Syndrome (ARDS).” This was a multicenter, multinational, randomized, double-blind, placebo-controlled clinical study to determine efficacy and safety of 9 mg FC Compound 17ya for the treatment of hospitalized moderate to severe COVID-19 adult patients who are at high risk for ARDS. Subjects were randomized in a 2:1 fashion to the Compound 17ya and placebo groups, respectively. The study was conducted in the United States, Argentina, Bulgaria, Brazil, Colombia, and Mexico between 19 May 2021 and 3 Jun. 2022.

Key inclusion criteria for this study were the following: (1) subjects ≥18 years of age and documented SARS-CoV-2 infection by polymerase chain reaction test; (2) patients with: WHO Ordinal Scale for Clinical Improvement score of 4 at high risk for ARDS who had at least 1 of the known comorbidities for being at high risk, such as: asthma [moderate to severe]; chronic lung disease; diabetes; hypertension; severe obesity [body mass index ≥40]; 65 years of age or older; primarily resided in a nursing home or long-term care facility; or immunocompromised; patients with WHO Ordinal Scale for Clinical Improvement score of 5 or 6 regardless of presence of comorbidities; or (3) peripheral capillary oxygen saturation (SpO2) ≤94% on room air at Screening.

Key exclusion criteria were the following: (1) pregnant or breastfeeding; (2) required ventilation plus additional organ support—pressors, renal replacement therapy (RRT), extracorporeal membrane oxygenation (ECMO) (WHO Ordinal Scale for Clinical Improvement—Score of 7). Note: short-term as-needed use of pressors was allowed; (3) moderate to severe renal impairment; or (4) Hepatic impairment.

Standard of care (SOC) for the treatment of SARS-CoV-2 infection (COVID-19) in hospitalized adult patients was allowed in the study. SOC varied by region and was accounted for in the case record forms. Randomization was stratified by baseline WHO Ordinal Scale for Clinical Improvement score of 4 (supplemental oxygen), 5 (NIV or high-flow oxygen) and 6 (mechanical ventilation) such that subjects with a WHO Ordinal Scale of 4, 5 and 6 at baseline were approximately equally distributed between the treatment groups. The WHO Ordinal Scale for Clinical Improvement is a scale that is commonly used to measure clinical improvement among clinical trial participants including studies in COVID-19.

The study required that each subject in the study receive a 9 mg daily oral (or via nasogastric tube) dose of Compound 17ya or placebo for up to 21 days (Day 21) or until the patient was discharged from the hospital (whichever came first) with efficacy and safety follow up continuing to Day 60 of the study. Selected clinical sites for this confirmatory Phase 3 COVID-19 Compound 17ya study were inspected by FDA in August 2022 and were found to be compliant with current Good Clinical Practices (GCP) guidelines.

Endpoints for Efficacy Evaluation

The endpoints for efficacy evaluation in Study A and Study B are presented side-by-side in Table 10, below. It is important to note that the primary efficacy endpoints for these studies were objective and not subject to interpretation or bias: In Study A, the primary efficacy endpoint was the proportion of subjects alive and free of respiratory failure at Day 29. In Study B, the primary efficacy endpoint was the proportion of subjects that die on study (up to Day 60).

TABLE 10

| Type | Study A | Study B |
|---|---|---|
| Primary | Proportion of subjects that were alive without | Proportion of subjects that die on study (up to |
| | respiratory failure at Day 29 | Day 60) |
| Secondary | 1. WHO Ordinal Scale for Clinical Improvement | 1. The proportion of subjects that are alive |
| | (8-point ordinal scale) | without respiratory failure at Day 15, Day 22, |
| | 2. Proportion of subjects that were Grade 5, 6, 7, | and Day 29. Day 29 is the key secondary |
| | or 8 on the WHO Ordinal Scale for Clinical | endpoint. Respiratory failure is defined as |
| | Improvement at Day 15 | endotracheal intubation and mechanical |
| | 3. Proportion of subjects that were Grade 5, 6, 7, | ventilation, extracorporeal membrane |
| | or 8 on the WHO Ordinal Scale for Clinical | oxygenation, high-flow nasal cannula oxygen |
| | Improvement at Day 22 | delivery, noninvasive positive pressure |
| | 4. Proportion of subjects that were Grade 5, 6, 7, | ventilation, clinical diagnosis of respiratory |
| | or 8 on the WHO Ordinal Scale for Clinical | failure with initiation of none of these |
| | Improvement at Day 29 | measures only when clinical decision making |
| | 5. Change in mean WHO Ordinal Scale for | is driven solely by resource limitation. |
| | Clinical Improvement at Day 15 | 2. Days in ICU |
| | 6. Change in mean WHO Ordinal Scale for | 3. WHO Ordinal Scale for Clinical Improvement |
| | Clinical Improvement at Day 22 | change from baseline to Day 15, Day 22, and |
| | 7. Change in mean WHO Ordinal Scale for | Day 29 |
| | Clinical Improvement at Day 29 | 4. Days on mechanical ventilation |
| | 8. Proportion of subjects with normalization of | 5. Days in hospital |
| | fever and oxygen saturation through Day 15, | 6. Proportion of subjects that die on study at Day |
| | Day 22, and Day 29 | 15, Day 22, and Day 29. |
| | 9. Days on mechanical ventilation | 7. Change from baseline in viral load (baseline to |
| | 10. Percentage of subjects discharged from hospital | Day 9) |
| | by Day 15 (and Day 22) | |
| | 11. All-cause mortality at Day 15, Day 22, Day 29, | |
| | and Day 60 | |
| | 12. Proportion of subjects alive and free of | |
| | respiratory failure at Day 15 and Day 22 | |
| | 13. Proportion of subjects alive and discharged | |
| | from the intensive care unit (ICU) at Day 15, | |
| | Day 22, and Day 29 | |
| | 14. Proportion of subjects alive and discharged | |
| | from hospital at Day 15, Day 22, and Day 29 | |
| | 15. Days in ICU | |
| | 16. Days in hospital | |
| | 17. Proportion of subjects on mechanical | |
| | ventilation at Day 15, Day 22, and Day 29 | |

Efficacy Results

Study A

In this Phase 2 study 39 subjects were randomized in a 1:1 fashion and received 18 mg PIC of Compound 17ya (19 subjects) or matching placebo (20 subjects), orally or through nasogastric tube, daily for up to 21 days or until the subject was discharged from the hospital, whichever came first. Overall, 33 subjects (84.6%) completed the study, with 6 subjects discontinuing prematurely. The mean (SD) treatment exposure was comparable in both the treatment groups: 9.0 (6.64) days in the Compound 17ya group and 11.2 (6.74) days in the placebo group.

The effective dose of Compound 17ya (18 mg PIC) appeared to minimize the frequency and severity of COVID-19 virus infection and the lethal respiratory adverse effects of the virus compared to placebo. Although Study A was a proof-of-concept study, Compound 17ya was shown to have a clinical beneficial effect in hospitalized adult patients with COVID-19 who were at high risk for ARDS. Data from Study A were used as hypothesis-generating for the design of the Phase 3 protocol of Study B.

Efficacy by Primary Variable in Study A

In Study A, the primary efficacy endpoint was the proportion of subjects alive and free of respiratory failure at Day 29. A higher proportion of subjects were alive without respiratory failure at all the time points in the Compound 17ya group as compared to the placebo group. Responders were subjects who were discharged from the hospital or had Grade 3 or 4 on the WHO Ordinal Scale for Clinical Improvement at the visit. At Day 29, a higher proportion of subjects were alive without respiratory failure in the Compound 17ya group (89.5%) as compared to the placebo group (70%). The results are shown in Table 11.

Logistic regression for the proportion of subjects alive and free of respiratory failure by visit (see Table 12) showed that at Day 29, the odds ratio between the Compound 17ya 18 mg PC vs Placebo groups was 2.58 (95% CI: 0.37, 18.07; p value=0.3394).

TABLE 11
Study A: Primary Efficacy Endpoint-Proportion of Subjects Alive and
Free of Respiratory Failure by Visit (ITT Population)

| | | Compound 17ya | Placebo |
|---|---|---|---|
| | | (N = 19) | (N = 20) |
| Visit | Responder Status | n (%) | n (%) |
| Day 15 | Responder^{a} | 16 (84.2) | 13 (65.0) |
| | Non-Responder^{b} | 3 (15.8) | 7 (35.0) |
| Day 22 | Responder^{a} | 16 (84.2) | 14 (70.0) |
| | Non-Responder^{b} | 3 (15.8) | 6 (30.0) |
| Day 29 | Responder^{a} | 17 (89.5) | 14 (70.0) |
| | Non-Responder^{b} | 2 (10.5) | 6 (30.0) |

^{a}Responders were subjects who had been discharged from the hospital or had Grade 0-4 on the WHO Ordinal Scale for Clinical Improvement at the visit.
^{b}Non-responders were subjects who died before the visit or had Grade 5-8 on the WHO Ordinal Scale for Clinical Improvement on the day of the visit.
Abbreviations: N = number of subjects in the randomized set; n = number of subjects in the specific category; WHO = World Health Organization

TABLE 12
Study A: Primary Efficacy Endpoint-Logistic Regression
for the Proportion of Subjects Alive and
Free of Respiratory Failure at Day 29 (ITT Population)

| | Degrees of | | |
|---|---|---|---|
| Effect | Freedom | Chi-Square | p-value |
| Treatment | 1 | 0.91 | 0.3394 |
| Study Site | 4 | 2.03 | 0.7302 |
| Remdesivir Use | 1 | 0.25 | 0.6152 |
| Dexamethasone Use | 1 | 0.01 | 0.9235 |
| Baseline WHO Scale | 1 | 4.23 | 0.0396 |
| Treatment | Odds | 95% CI | p-value |
| Compound 17ya 18 mg PIC | 6.75 | (1.05, 43.57) | 0.0448 |
| Placebo | 2.61 | (0.49, 13.92) | 0.2601 |
| | Odds | | |
| Treatment Comparison | Ratio | 95% CI | p-value |
| Compound 17ya 18 mg | 2.58 | (0.37, 18.07) | 0.3394 |
| PIC vs. Placebo | | | |

Responders were subjects who had been discharged from the hospital or had Grade 0-4 on the WHO Ordinal Scale for Clinical Improvement at the visit.
Non-responders were subjects who died before the visit or had Grade 5-8 on the WHO Ordinal Scale for Clinical Improvement on the day of the visit.
Abbreviations: CI = confidence interval; ITT = Intent-to-Treat; WHO = World Health Organization

Efficacy by Secondary Variables in Study A

The key secondary endpoint outcomes for this study were the following: (1) an 82% relative reduction (25% absolute reduction) in mortality at Day 60 was observed in the Compound 17ya treated group (1/19 patients, 5.3%) compared to the placebo group (6/20, 30%); (2) a 73% relative reduction in days in the ICU was observed in the Compound 17ya treated group (2.6±5.8 days) compared to placebo group (9.6±12.4); (3) a 78% relative reduction in days on mechanical ventilation was observed in the Compound 17ya treated group (1.2±6.1 days) compared to placebo group (5.1±11.2 days).

Study B

Study B was a Phase 3 pivotal efficacy and safety study of hospitalized adult patients with moderate to severe COVID-19 who were at high risk for ARDS. The primary endpoint for the study was the proportion of patients that died up to day 60. Approximately 210 patients were planned for enrollment with randomization of patients to Compound 17ya and matching placebo in a 2:1 fashion. At a planned interim analysis for the first 150 patients randomized into the study (Interim Analysis Intent-to-Treat [IA ITT] population) an Independent Data Monitoring Committee unanimously voted to stop the study early for evidence of clear clinical benefit and noted that no safety concerns were observed in the study. In the IA ITT population, there were 150 patients (98 patients received Compound 17ya 9 mg capsule and 52 patients received Placebo). In the Full Study population there were 204 patients (134 patients received Compound 17ya 9 mg capsule and 70 received Placebo).

At the end of the Phase 3 study there were 6 patients for whom mortality status at Day 60 was unknown (4 patients in the VERU-111 group and 2 patients in the Placebo group). For each of these 6 cases the clinical study sites made multiple attempts to reach the patient at Day 60 (phone calls to the patient and emergency contacts, registered mail) but these attempts were unsuccessful. In addition, attempts were made to confirm the patients' mortality status through available medical records, public records, and review of local obituaries, etc. Ultimately, the sites were not able to determine whether these patients were alive or deceased at Day 60 and therefore this data is confirmed to be missing.

Efficacy by Primary Variable in Study B

Treatment with Compound 17ya resulted in a clinically meaningful and statistically significant reduction in mortality compared to placebo. A summary of the primary endpoint is provided for the Interim Analysis (IA) ITT population in Table 13, and for the overall ITT population in Table 14.

TABLE 13
Study B: Proportion of Subjects That Died Prior To Day 60
(IA ITT Population)

| | Compound | | % Relative | % Absolute |
|---|---|---|---|---|
| Efficacy Parameter | 17 ya | Placebo | Reduction | Reduction |
| Proportion of | 19/94 | 23/51 | 55.2% | 24.9% |
| patients that | (20.2%) | (45.1%) | | |
| died by day | | | | |
| 60-IA ITT | | | | |
| population | | | | |

| | Odds Ratio | 95% CI | p-value |
|---|---|---|---|
| Compound 17ya vs. | 3.23 | (1.45, 7.22) | 0.0042 |
| Placebo | | | |

Model effects are displaying median of the p-values for imputed analyses. Odds Ratio and associated 95% Confidence Interval (CI) is presented for the probability of survival at Day 60. An odds ratio > 1 indicates benefit in Compound 17ya group. Multiple imputation used for missing vital status at Day 60. Imputation model included treatment, region, sex, remdesivir use, dexamethasone use and WHO strata, and additionally subject's discharge status and early treatment discontinuation status.
Abbreviations: CI = confidence interval; ITT = Intent-to-Treat

TABLE 14
Study B: Proportion of Subjects That Died Prior to Day 60
(ITT Population)

| | Compound | | % Relative | % Absolute |
|---|---|---|---|---|
| Efficacy Parameter | 17ya | Placebo | Reduction | Reduction |
| Proportion of patients | 25/130 | 27/68 | 51.6% | 20.5% |
| that died | (19.2%) | (39.7%) | | |
| by day 60-ITT population | | | | |
| | Odds Ratio | 95% CI | p-value | |
| Compound 17ya vs. | 2.77 | (1.37, | 0.0046 | |
| Placebo | | 5.60) | | |

Model effects are displaying median of the p-values for imputed analyses. Odds Ratio and associated 95% Confidence Interval (CI) is presented for the probability of survival at Day 60. An odds ratio > 1 indicates benefit in Compound 17ya group. Multiple imputation used for missing vital status at Day 60. Imputation model included treatment, region, sex, remdesivir use, dexamethasone use and WHO strata, and additionally subject's discharge status and early treatment discontinuation status.
Abbreviations: CI = confidence interval; ITT = Intent-to-Treat

Kaplan-Meier Analysis (ITT Population)

The probability of dying, based on Kaplan-Meier estimates, was numerically lower for Compound 17ya 9 mg versus placebo at each assessed time point (see Table 15, below). Treatment comparisons using log-rank and Wilcoxon χ² tests (Compound 17ya versus placebo) were statistically significant in favor of Compound 17ya:

Log-rank χ²: 9.639, P=0.0019

Wilcoxon χ²: 9.307, P=0.0023

TABLE 15
Study B: Kaplan-Meier Estimates for Overall Mortality (ITT Population)

| | | | Absolute Risk Reduction |
|---|---|---|---|
| | | | (Placebo versus |
| | Compound 17ya 9 mg | Placebo | Compound 17ya 9 mg) |
| | (N = 134) | (N = 70) | Estimate (95% CI) |
| Number (%) of patients who died | 25 (18.7) | 27 (38.6) | — |
| Number (%) of patients censored | 109 (81.3) | 43 (61.4) | — |
| Kaplan-Meier Estimates | | | |
| 25th percentile (95% CI) | NA (37.0, NA) | 26.0 (14.0, 40.0) | — |
| Median (95% CI) | NA (NA, NA) | NA (41.0, NA) | — |
| 75th percentile (95% CI) | NA (NA, NA) | NA (NA, NA) | — |
| Probability of dying by Day 15 (95% CI) | 8.4 (4.7, 14.6) | 21.6 (13.6, 33.2) | 13.2 (2.4, 24.0) |
| Probability of dying by Day 22 (95% CI) | 13.0 (8.3, 20.0) | 23.0 (14.8, 34.8) | 10.0 (−1.4, 21.5) |
| Probability of dying by Day 29 (95% CI) | 15.3 (10.1, 22.7) | 28.9 (19.7, 41.2) | 13.7 (1.3, 26.0) |
| Probability of dying by Day 45 (95% CI) | 18.3 (12.7, 26.1) | 39.3 (28.9, 51.9) | 21.0 (7.6, 34.3) |
| Probability of dying by Day 60 (95% CI) | 19.1 (13.3, 27.0) | 39.3 (28.9, 51.9) | 20.2 (6.8, 33.6) |

Abbreviations: CI = confidence interval; ITT = Intent-to-Treat; NA = not applicable.

There was clear separation between the Compound 17ya 9 mg and placebo Kaplan-Meier curves for time to death (Error! Reference source not found).

Overall, the following conclusions are made for the primary analysis. (1) The percentage of patients in the ITT Set (all randomized subjects) who had died up to Day 60 was lower in the Compound 17ya 9 mg group compared with placebo (19.2% and 39.7%, respectively). A similar result was noted in the Safety Set (18.0% and 38.8%, respectively) and the modified Intent to Treat (mITT) Set (18.1% and 38.8%, respectively). The Safety Set consisted of all randomized subjects who received at least one dose of study medication and the mITT Set consisted of all randomized subjects who completed the efficacy portion of the trial and who did not have any major protocol violations. (2) The odds ratio (OR) for survival at Day 60 in the ITT Set was statistically significant in favor of Compound 17ya (OR: 2.77 [95% CI: 1.37, 5.60], P=0.0046). A similar result was noted in the Safety Set (2.92 [95% CI: 1.43, 5.96], P=0.0033) and the mITT Set (OR: 2.88 [95% CI: 1.41, 5.88], P=0.0037). When logistic regression analysis was repeated using an identity link function, the OR for mortality was statistically significant in favor of Compound 17ya (0.19 [95% CI: 0.06, 0.31], P=0.0029). (3) The Kaplan-Meier curves for overall mortality in the ITT Set showed clear separation between the Compound 17ya 9 mg and placebo groups. (4) In a Cox proportional hazards model, the hazard ratio for overall mortality in the ITT Set was statistically significant in favor of Compound 17ya (hazard ratio: 0.43 [95% CI: 0.25, 0.75], P=0.0029). (5) A sensitivity analysis using the tipping-point approach was also conducted to assess the robustness of the primary analysis approach and found that the results were consistently in favor of Compound 17ya versus placebo. The tipping-point analysis considered the full range of possible response rates in the six patients with missing data in the primary analysis. Multiple imputation was used for each pair of response rates under consideration. Both the imputation model and the analysis model incorporated the covariates used in the primary analysis. The tipping point analysis was done by systematically changing the assumed response rates from 0 to 100% in a stepwise manner. The imputation was performed independently within the 2 treatment groups so that, in the most extreme case, the imputed response rate was 0% (all missing patients dead) in the Compound 17ya arm and 100% (all missing patients alive) in the placebo arm. In the extreme case, the p-value remained statically significant at p=0.0086.

Efficacy of Primary Variable—Prespecified Subgroup Analyses in Study B

Standard of Care

In Study B, patients were permitted to receive COVID-19 standard of care treatments including dexamethasone and remdesivir. Analyses were conducted to determine if there were differences in the mortality in patients who did or did not receive standard of care treatments. The following tables describe the number of subjects by arm (including deaths on-study) for: Subjects that initiated treatment with remdesivir prior to Day 1 of the study (Table 16); Subjects that initiated treatment with dexamethasone prior to Day 1 of the study (Table 17); Subjects that received COVID-19 vaccine (Table 18); and Subjects that received a US authorized COVID-19 vaccine (Table 19).

TABLE 16
Study B: Subjects Who Initiated Remdesivir Prior To or
On Day 1 of the Study

| | | | Absolute | | |
|---|---|---|---|---|---|
| | | | Change | Relative | |
| | Compound | Placebo | (percentage | Change | |
| | 17ya | | points) | (%) | p-value |
| NO | 92 | 51 | | | |
| Deaths (%) | 16 (17.4%) | 17 (33.3%) | −15.9 | −47.7% | |
| YES | 38 | 17 | | | |
| Deaths (%) | 9 (23.7%) | 10 (58.8%) | −35.1 | −59.7% | 0.0283 |

NOTE:
The mortality presented in this table is up to Day 60.

TABLE 17
Study B: Subjects Who Initiated Dexamethasone
Treatment Prior To or On Day 1 of the Study

| | | | Absolute | | |
|---|---|---|---|---|---|
| | | | Change | | |
| | Compound | | (percentage | Relative | |
| | 17ya | Placebo | points) | Change (%) | p-value |
| NO | 26 | 15 | | | |
| Deaths (%) | 1 (3.8%) | 5 (33.3%) | −29.5 | −88.6% | |
| YES | 104 | 53 | | | |
| Deaths (%) | 24 (23.1%) | 22 (41.5%) | −18.4 | −44.3% | 0.0367 |

NOTE:
The mortality presented in this table is up to Day 60.

TABLE 18
Study B: Subjects Who Were Vaccinated (1, 2, or 3 shots)

| | | | Absolute | |
|---|---|---|---|---|
| | | | Change | |
| | Compound | | (percentage | Relative |
| | 17ya | Placebo | points) | Change (%) |
| NO | 83 | 41 | | |
| Deaths (%) | 15 (18.1%) | 18 (43.9%) | −25.8 | −58.8% |
| YES | 47 | 27 | | |
| Deaths (%) | 10 (21.3%) | 9 (33.3%) | −12.0 | −36.0% |

NOTE:
The mortality presented in this table is up to Day 60.

TABLE 19
Study B: Subjects Who Were Vaccinated with a US Authorized Vaccine

| | | | Absolute | |
|---|---|---|---|---|
| | | | Change | |
| | Compound | | (percentage | Relative |
| | 17ya | Placebo | points) | Change (%) |
| NO | 118 | 61 | | |
| Deaths (%) | 22 (18.6%) | 23 (37.7%) | −19.1 | −50.7% |
| YES | 12 | 7 | | |
| Deaths (%) | 3 (25.0%) | 4 (57.1%) | −32.1 | −56.2% |

NOTE:
The mortality presented in this table is up to Day 60.

Compound 17ya shows a statistically significant and clinically meaningful reduction in mortality (−59.7% relative reduction) in patients who initiated remdesivir treatment prior to initiation of study drug. There is also a clinically meaningful reduction in mortality (−47.7% relative reduction) in patients who did not initiate treatment with remdesivir prior to initiation of study drug. Based on these data, Veru proposes that Compound 17ya may be administered with or without prior initiation of remdesivir treatment. Compound 17ya may be first line therapy in this patient population.

Compound 17ya shows a statistically significant and clinically meaningful reduction (−44.6% relative reduction) in mortality in patients who initiated dexamethasone treatment prior to initiation of study drug. There is also a clinically meaningful reduction in mortality (−88.6% relative reduction) in patients who did not initiate treatment with dexamethasone prior to initiation of study drug. Based on these data, the Sponsor proposes that Compound 17ya may be administered with or without prior initiation of dexamethasone treatment. Compound 17ya may be first line therapy or part of first line therapy (coadministration with corticosteroid therapy) in this patient population.

A clinically meaningful reduction in mortality was observed in vaccinated patients (any vaccine), unvaccinated patients, vaccinated patients (US authorized vaccine), and patients that were not vaccinated with a US authorized vaccine.

WHO Ordinal Scale Score at Baseline

In Study B randomization was stratified by WHO Ordinal Scale at randomization. It is noted that some patients showed disease progression after randomization and prior to first dose. The data presented here represent the WHO score on Day 1 (not randomization). Analyses were conducted to determine the mortality in patients with WHO 4 (supplemental oxygen/passive oxygen), WHO 5 (NIV or high-flow oxygen), and WHO 6 (mechanical ventilation). The following table describes the number of subjects by arm (including deaths on-study) for subjects by WHO Ordinal Score at baseline (Day 1).

TABLE 20
Study B: subjects by WHO Status on Day 1 of the Study

| | | | Absolute | | |
|---|---|---|---|---|---|
| | | | Change | | |
| | Compound | | (percentage | Relative | |
| | 17ya | Placebo | points) | Change (%) | p-value |
| WHO 4 | 58 | 29 | | | |
| Deaths (%) | 3 (5.2%) | 8 (27.6%) | −22.4 | −81.2% | 0.0090 |
| WHO 5 | 60 | 31 | | | |
| Deaths (%) | 20 (33.3%) | 15 (48.4%) | −15.1 | −31.2% | 0.3206 |
| WHO 6 | 12 | 8 | | | |
| Deaths (%) | 2 (16.7%) | 4 (50.0%) | −33.3 | −66.7% | 0.2100 |

NOTE:
The mortality presented in this table is up to Day 60.

Compound 17ya shows a clinically meaningful reduction in mortality in each of the WHO Ordinal Scores at baseline. As discussed elsewhere in this document, the Sponsor notes that the patient population enrolled in this study represented patients that have significant progression of COVID infection and/or have a high risk for further progression. At the request of FDA, the inclusion/exclusion criteria for Study B were specifically chosen to enroll the patients who were in the highest risk population.

Efficacy of Primary Variable—Sensitivity Analyses to Test for Robustness of Data

Subgroup Analyses

The following tables show the absolute and relative reduction in risk of mortality by Day 60 compared to placebo by subgroup for the Full Study population (all 204 subjects enrolled). Table 21 examines various subgroups in the study based on demographic, baseline characteristics, and dominant COVID-19 variant (considering 3 plausible cut-offs for the delta-omicron variant dominance switch: 15 Dec. 2021, 15 Jan. 2022, and 15 Feb. 2022). Based on the mechanism of action of Compound 17ya (disruption and depolymerization of microtubules of the host cells) it is expected that the effects of Compound 17ya are both virus-independent and variant-independent. Table 22 examines various subgroups in the study based on comorbidities that are known to increase risk of ARDS. Table 23 examines various subgroups in the study who received prior vaccination or certain types of COVID-19 standard of care treatments.

These sensitivity analyses show that there is a reduction in mortality by Day 60 in all subgroups receiving Compound 17ya compared to placebo. These results further support the robustness of the overall primary endpoint analysis as similar mortality reductions with Compound 17ya treatment are observed in all subgroups concerning demographics, baseline characteristics, SARS-CoV-2 variant, comorbidities, vaccination status, and COVID-19 standard of care. In the “backward logistic regression” analysis where the effect of multiple variables and combination of variables was assessed, the effectiveness of Compound 17ya in reduction in mortality is maintained (p=0.0050).

TABLE 21
Study B: Absolute and Relative Reduction in Risk of Mortality by Day 60 Compared to
Placebo in Subgroups Based on Demographics, Baseline Characteristics, and SARS-CoV-2 Variant

| | Compound | | Absolute | Relative |
|---|---|---|---|---|
| Subgroup | 17ya | Placebo | difference | difference |
| Males | 15/88 (17.0%) | 19/43 (44.2%) | −27.1% | −61.4% |
| Females | 10/42 (23.8%) | 8/25 (32.0%) | −8.2% | −25.6% |
| Age <60 years | 4/44 (9.1%) | 7/20 (35.0%) | −25.9% | −74.0% |
| Age ≥60 years | 21/86 (24.4%) | 20/48 (41.7%) | −17.2% | −41.4% |
| WHO 4 | 3/58 (5.2%) | 8/29 (27.6%) | −22.4% | −81.2% |
| WHO 5 | 20/60 (33.3%) | 15/31 (48.4%) | −15.1% | −31.2% |
| WHO 6 | 2/12 (16.7%) | 4/8 (50.0%) | −33.3% | −66.6% |
| US | 12/42 (28.6%) | 13/23 (56.5%) | −27.9% | −49.4% |
| OUS | 13/88 (14.8%) | 14/45 (31.1%) | −16.3% | −52.4% |
| Delta Variant (randomized prior | 13/48 (27.1%) | 12/26 (46.2%) | −19.1% | −41.3% |
| to Dec. 15, 2021) | | | | |
| Omicron Variant (randomized | 12/82 (14.6%) | 15/42 (35.7%) | −21.1% | −59.1% |
| on or after Dec. 15, 2021) | | | | |
| Omicron Variant (randomized | 7/61 (11.5%) | 9/32 (28.1%) | −16.6% | −59.1% |
| on or after Jan. 15, 2022) | | | | |
| Omicron Variant (randomized | 2/17 (11.8%) | 3/12 (25.0%) | −13.2% | −52.8% |
| on or after Feb. 15, 2022) | | | | |

TABLE 22
Study B: Absolute and Relative Reduction in Risk of Mortality by Day 60 Compared to
Placebo in Subgroups Based on Comorbidities Known to Increase Risk of ARDS

| | Compound | | Absolute | Relative |
|---|---|---|---|---|
| Subgroup | 17ya | Placebo | difference | difference |
| Hypertension | 20/84 (23.8%) | 17/45 (37.8%) | −14.0% | −37.0% |
| Pneumonia | 16/76 (21.1%) | 15/44 (34.1%) | −13.0% | −38.1% |
| Diabetes | 12/45 (26.7%) | 12/28 (42.9%) | −16.2% | −37.8% |
| ≥65 years of age | 16/65 (24.6%) | 16/40 (40.0%) | −15.4% | −38.5% |
| Severe Respiratory Issues* | 4/36 (11.1%) | 6/13 (46.2%) | −35.1% | −76.0% |
| Severe Obesity (BMI ≥40) | 3/23 (13.0%) | 3/6 (50.0%) | −37.0% | −74.0% |
| Hypertension + 3 other | 9/40 (22.5%) | 6/16 (37.5%) | 15.0% | −40.0% |
| comorbidities | | | | |
| Pneumonia + 3 other | 8/31 (25.8%) | 5/15 (33.3%) | −7.5% | −22.5% |
| comorbidities | | | | |
| ≥65 yoa + 3 other comorbidities | 5/28 (17.9%) | 5/13 (38.5%) | −20.6% | −53.5% |
| ≥4 comorbidities | 10/43 (23.2%) | 6/18 (33.3%) | −10.1% | −30.2% |
| ≥3 comorbidities | 16/73 (21.9%) | 14/41 (34.1%) | −12.2% | −35.8% |
| ≥2 comorbidities | 25/106 (23.6%) | 23/58 (39.7%) | −16.1% | −40.5% |

Severe respiratory issues = Asthma, Bronchiectasis, Bronchitis chronic, Chronic obstructive pulmonary disease, Interstitial lung disease, Pulmonary fibrosis, and/or Pulmonary sarcoidosis

TABLE 23
Study B: Absolute and Relative Reduction in Risk of Mortality by Day 60 Compared to
Placebo in Subgroups Based on Vaccination Status and COVID-19 Standard of Care Treatments

| | Compound | | Absolute | Relative |
|---|---|---|---|---|
| Subgroup | 17ya | Placebo | difference | difference |
| Vaccinated | 10/47 (21.3%) | 9/27 (33.3%) | −12.1% | −36.2% |
| Unvaccinated | 15/83 (18.1%) | 18/41 (43.9%) | −25.8% | −58.8% |
| Remdesivir YES | 9/38 (23.7%) | 10/17 (58.8%) | −35.1% | −59.7% |
| Remdesivir Treatment co- | 5/27 (18.5%) | 4/8 (50.0%) | −31.5% | −63.0% |
| administered on study (from Day | | | | |
| 1 of the study) | | | | |
| Remdesivir NO | 16/92 (17.4%) | 17/51 (33.3%) | −15.9% | −47.8% |
| Dexamethasone YES | 24/104 (23.1%) | 22/53 (41.5%) | −18.4% | −44.4% |
| Dexamethasone Treatment co- | 20/103 (19.4%) | 19/49 (38.8%) | −19.4% | −49.9% |
| administered on study (from Day | | | | |
| 1 of the study) | | | | |
| Dexamethasone NO | 1/26 (3.8%) | 5/16 (31.3%) | −27.4% | −87.7% |
| Any Systemic Corticosteroid | 25/127 (19.7%) | 25/65 (38.5%) | −18.8% | −48.8% |
| Tocilizumab YES | 4/8 (50.0%) | 6/7 (85.7%) | −35.7% | −41.7% |
| Tocilizumab NO | 21/122 (17.2%) | 21/61 (34.4%) | −17.2% | −50.0% |
| JAK inhibitor YES | 1/9 (11.1%) | 3/8 (37.5%) | −26.4% | −70.4% |
| JAK inhibitor NO | 24/121 (19.8%) | 24/60 (40.0%) | −20.2% | −50.4% |

Backward Logistic Regression

To assess the effect (and combination of effects) of various factors in the study on the primary endpoint of the study (mortality by Day 60), a backward logistic regression with stepwise procedure was conducted. The following factors were included in this analysis: treatment, region, sex, remdesivir use at baseline, dexamethasone use at baseline, WHO scale score at randomization, selected respiratory issues (Asthma, Bronchiectasis, Bronchitis chronic, Chronic obstructive pulmonary disease, Interstitial lung disease, Pulmonary fibrosis, Pulmonary sarcoidosis), asthma, history of heart failure, diabetes, severe obesity (BMI ≥40 kg/m²), age ≥65 years, and ≥3 of selected respiratory issues/history of heart failure/diabetes/BMI ≥40/age ≥65.

The results of this stepwise logistic regression are provided in Table 24 and show that when these factors are taken into account, they have very little effect on the overall statistical conclusion of the primary endpoint. Compound 17ya has a highly statistically significant effect on reducing mortality by Day 60 compared to placebo in this analysis (p=0.0050; odds ratio: 2.93 [95% confidence interval: 1.38, 6.22]). Therefore, it is concluded that any small potential imbalances in these study factors do not appear to have a singular or combined effect on the observed benefit of Compound 17ya in the primary study endpoint, reduction in death by Day 60.

TABLE 24
Study B: Backward Logistic Regression for Proportion of Subjects Alive by Day 60 (ITT Set)

| Effect | Degrees of Freedom | Chi-Square | p-value |
|---|---|---|---|
| Treatment | NA | NA | 0.0042 |
| Region (North America / South America / | NA | NA | 0.0050 |
| Europe) | | | |
| Sex (Male / Female) | NA | NA | 0.0636 |
| Remdesivir use (No / Yes) | NA | NA | 0.3647 |
| Dexamethasone use (No / Yes) | NA | NA | 0.0571 |
| WHO Scale strata for randomization (4, 5, 6) | NA | NA | 0.0423 |
| Diabetes | NA | NA | 0.1059 |
| Age >=65 years | NA | NA | 0.0010 |
| >=3 of ‘Selected Respiratory Issues’, ‘History of | NA | NA | 0.3613 |
| Heart Failure’, ‘Diabetes’, ‘BMI >=40’, | | | |
| ‘Age >=65’ | | | |
| Treatment | Odds | 95% CI | p-value |
| Compound 17ya 9 mg | 6.40 | (2.70, 15.20) | <0.0001 |
| Placebo | 2.18 | (0.89, 5.36) | 0.0883 |
| Treatment Comparison | Odds Ratio | 95% CI | p-value |
| Compound 17ya 9 mg vs. Placebo | 2.93 | (1.38, 6.22) | 0.0050 |

Responders are subjects who are alive at the time point.

Non-responders are subjects who died before the time point.

Missing vital status was handled using multiple imputation methods. Imputation model included treatment, region, sex, remdesivir use at baseline, dexamethasone use at baseline and WHO strata, and additionally subject's discharge status and early treatment discontinuation status.

Analysis model was selected using stepwise logistic regression on the observed data using entry criteria 0.4 and stay criteria 0.5.

Terms included in the stepwise procedure were: treatment, region, sex, remdesivir use at baseline, dexamethasone use at baseline, WHO strata, selected respiratory issues, asthma, history of heart failure, diabetes, severe obesity (BMI >=40 kg/m2), age >=65 years and >=3 of (selected respiratory issues, history of heart failure, diabetes, BMI >=40, age >=65).

Model effects are displaying median of the p-values for the imputed analyses.

An odds ratio >1 indicates benefit in the Compound 17ya group.

Efficacy by Secondary Variables

Analyses of secondary endpoints in the Phase 3 COVID-19 Compound 17ya study were consistently in favor of Compound 17ya versus placebo, including: the proportion of patients dead or with respiratory failure at Days 15, 22, 29 and 60 (Table 25) [non-responders in the analysis ‘alive without respiratory failure’; this endpoint is analogous to the Phase 2 primary endpoint]; Days in the ICU (Table 26); Days on mechanical ventilation (Table 27); Days in the hospital (Table 28); and Viral load (Table 28).

TABLE 25
Study B: Proportion of Patients Dead or with Respiratory Failure [Non-Responders in the
Alive Without Respiratory Failure Analysis] (ITT population)

| | | | Absolute | % Relative | |
|---|---|---|---|---|---|
| | Compound 17ya | Placebo | change | change | p-value |
| Day 15 | 37/131 (28.2%) | 29/69 (42.0%) | −13.8 | −32.9% | 0.0863 |
| Day 22 | 35/131 (26.7%) | 30/69 (43.5%) | −16.8 | −38.6% | 0.0269 |
| Day 29 | 34/130 (26.2%) | 30/68 (44.1%) | −17.9 | −40.6% | 0.0186 |
| Day 60 | 26/130 (20.0%) | 27/68 (39.7%) | −19.7 | −49.6% | 0.0066 |

The proportion of patients that died or had respiratory failure (non-responders in the analysis for ‘alive without respiratory failure’; this endpoint is analogous to the Phase 2 primary endpoint) at each time point assessed showed a clinical benefit in the Compound 17ya group compared to placebo. At the primary analysis day for this secondary endpoint, Day 29, there was a statistically significant (p=0.0186) reduction in treatment failures in the Compound 17ya group compared to placebo. Observationally, the proportion of treatment failures in the Compound 17ya group reduces at each subsequent time point from Day 15 to Day 29 while in the placebo group, the proportion of treatment failures increases over this time frame. The statistically significant (p=0.0066) benefit in the proportion of patients that were dead or with respiratory failure in the Compound 17ya group compared to placebo is maintained to Day 60.

TABLE 26
Study B: Days in ICU (ITT population)

| | n | Mean | SD | Median |
|---|---|---|---|---|
| Compound 17ya | 134 | 16.0 | 23.50 | 2.0 |
| Placebo | 70 | 26.3 | 28.11 | 9.0 |
| Treatment | | | | |
| comparison | LS mean | SE | 95% CI | p-value |
| | −9.9 | 3.44 | −16.7, −3.1 | 0.0045 |

NOTE:
in this analysis, the days in the ICU in patients that died on study is set at the worst possible outcome (60 days)

Treatment with Compound 17ya resulted in a statistically significant (p=0.0045) reduction in days in ICU by the protocol defined and FDA required analysis. Not presented in the table, without the imputation of worst possible outcome (60 days) for the patients that died on study, there is a 1.9 day reduction in days in the ICU in the Compound 17ya group (mean of 7.4 days) vs. the placebo group (mean of 9.3 days), representing a 20.4% relative reduction in actual days in the ICU in the Compound 17ya group compared to placebo. This finding shows that Compound 17ya treatment will reduce the burden on hospital and critical care staff in a surge of infections.

TABLE 27
Study B: Days on Mechanical Ventilation (ITT population)

| | n | Mean | SD | Median |
|---|---|---|---|---|
| Compound 17ya | 134 | 24.0 | 21.78 | 13.0 |
| Placebo | 70 | 31.0 | 24.61 | 16.5 |
| Treatment | | | | |
| comparison | LS mean | SE | 95% CI | p-value |
| | −6.3 | 3.13 | −12.4, −0.1 | 0.0463 |

Treatment with Compound 17ya resulted in a statistically significant (p=0.0038) reduction in days on mechanical ventilation by the protocol defined and FDA required analysis. Not presented in the table, without the imputation of worst possible outcome (60 days) for the patients that died on study, there is a 1.6 day reduction in days on mechanical ventilation in the Compound 17ya group (mean of 4.4 days) vs. the placebo group (mean of 6.0 days), representing a 26.7% relative reduction in actual days on mechanical ventilation in the Compound 17ya group compared to placebo. This finding shows that Compound 17ya treatment will reduce the burden on hospital and critical care staff in a surge of infections.

TABLE 28
Study B: Days in the Hospital (ITT population)

| | n | Mean | SD | Median |
|---|---|---|---|---|
| Compound 17ya | 134 | 24.0 | 21.78 | 13.0 |
| Placebo | 70 | 31.0 | 24.61 | 16.5 |
| Treatment | | | | |
| comparison | LS mean | SE | 95% CI | p-value |
| | −6.3 | 3.13 | −12.4, −0.1 | 0.0463 |

NOTE:
in this analysis, the days on mechanical ventilation in patients that died on study is set at the worst possible outcome (60 days)

Treatment with Compound 17ya resulted in a statistically significant (p=0.0463) reduction in days in the hospital by the protocol defined and FDA required analysis. Not presented in the table, without the imputation of worst possible outcome (60 days) for the patients that died on study, there is a 0.8 day increase in days in the hospital in the Compound 17ya group (mean of 16.2 days) vs. the placebo group (mean of 15.4 days). As defined, in this analysis, the patients that died are assigned the actual days they were in the hospital. One possible explanation for this result is that Compound 17ya treatment may cause a prolonged hospital stay in patients that would have otherwise died in the placebo group. This analysis shows that even with a 51.6% relative reduction in mortality, the actual average days in the hospital with no imputation for deaths is no different between the treatment groups.

TABLE 29
Study B: Viral load (ITT population)

| | | | | | % Relative |
|---|---|---|---|---|---|
| | | | | | Change from |
| | n | Mean | SD | Median | Baseline |
| Compound 17ya | | | | | |
| Baseline | 119 | 3222891.6 | 18760830.02 | 9380.0 | — |
| Change from BL to Last on-Study | 101 | −1383566.0 | 30515153.10 | −4422.0 | −42.9% |
| Change from BL to Day 9 only | 68 | −811345.0 | 36345622.22 | −6956.0 | −25.2% |
| Placebo | | | | | |
| Baseline | 64 | 2368764.6 | 12831731.51 | 3320.0 | — |
| Change from BL to Last on-Study | 52 | 9761507.2 | 83144880.94 | −834.5 | +412% |
| Change from BL to Day 9 only | 37 | 17500303.3 | 98692922.60 | −1527.0 | +739% |

The comparison of the effect on viral load by treatment group did not reach statistical significance due to the high variability in the measurement. This is not unexpected with the current technique and assays used to assess viral load. Mean viral load from baseline to last on-study assessment showed a 42.9% relative reduction in the Compound 17ya group compared to a 412% increase in the placebo group. In the Day 9 only assessment, the mean viral load decreased by 25.2% in the Compound 17ya group and increased by 739% in the placebo group. To minimize the effect of variability, a comparison of the median value at baseline and at Day 9 and last on-study assessment was conducted which shows a beneficial effect of Compound 17ya. Specifically, there is a 47.1% reduction in median viral load in the Compound 17ya group in the last on-study assessment compared to a 25.1% reduction in the placebo group. This represents an 87.6% relative reduction in the Compound 17ya group compared to placebo. In the Day 9 only assessment, there is a 74.2% reduction in median viral load in the Compound 17ya group compared to a 46.0% reduction in the median viral load in the placebo group. This represents a 61.3% relative reduction in median viral load in the Compound 17ya group compared to placebo. The observed reduction in viral load with Compound 17ya treatment is expected based on the antiviral mechanism of action of Compound 17ya.

Efficacy by Secondary Variables—Sensitivity Analyses to Test for Robustness of Data

A sensitivity analysis was conducted on the key secondary endpoints (regarding the total time of hospitalization, total time in the ICU, and total time on mechanical ventilation) to describe hospital-free survival days, ICU-free days, and mechanical ventilation-free days for each individual patient up to 60 days of follow-up. In this analysis, a patient contributed observations until day 60 or the time of censoring, whichever occurred first. ‘Days’ in this analysis were counted as alive and ‘not in the hospital,’ ‘not in the ICU,’ or ‘not on mechanical ventilation’ (three separate analyses). The method used for this analysis is generalized from the procedure reported in McCaw et al. (McCaw et al., “How to Quantify and Interpret Treatment Effects in Comparative Clinical Studies of COVID-19,” Annals of Internal Medicine, 2020, 173, 632-637), in which one can obtain the estimated mean of the hospitalization-free survival days, the mean of ICU-free survival days, and the mean of mechanical ventilation-free survival days for each treatment group (McCaw et al., 2020). One can then use the difference or ratio of the means from the two study arms to quantify the treatment effect. The results of these analyses are presented in the following tables: Hospital-Free Days (Alive and Not In The Hospital) (Table 30); ICU-Free Days (Alive and Not In The ICU) (Table 31); and Mechanical Ventilation-Free Days (Alive and Not On Mechanical Ventilation) (Table 32).

It was concluded from these sensitivity analyses that there were statistically significant and clinically meaningful increases in the Compound 17ya group compared to placebo for mean days alive and out of the hospital, mean days alive and not in the ICU, and mean days alive and not on mechanical ventilation. This analysis further confirmed the benefit of Compound 17ya in hospitalized patients with moderate to severe COVID-19 at high risk for ARDS.

TABLE 30
Study B: Hospital-Free Days (Alive and Not In The Hospital)

| | | | | Lower | Upper |
|---|---|---|---|---|---|
| | | Mean Days | SE | 95% CI | 95% CI |
| Placebo | | 28.0 | 2.88 | 22.4 | 33.6 |
| Compound 17ya | | 36.1 | 1.81 | 32.6 | 39.7 |
| | | | Lower | Upper | |
| Contrast Analysis | Estimate | SE | 95% CI | 95% CI | p-value |
| Absolute difference | 8.11 | 3.44 | 1.45 | 14.80 | 0.017 |
| (Compound 17ya − | | | | | |
| placebo) | | | | | |
| Ratio (Compound | 1.29 | 0.147 | 1.03 | 1.61 | 0.026 |
| 17ya/placebo) | | | | | |

CI: confidence interval; SE: standard error.

TABLE 31
Study B: ICU-Free Days (Alive and Not In The ICU)

| | | | Lower | Upper | |
|---|---|---|---|---|---|
| | Mean Days | SE | 95% CI | 95% CI | |
| Placebo | 34.2 | 3.14 | 28.0 | 40.3 | |
| Compound 17ya | 44.2 | 1.88 | 40.5 | 47.9 | |
| | | | Lower | Upper | |
| Contrast Analysis | Estimate | SE | 95% CI | 95% CI | p-value |
| Absolute difference | 10.0 | 3.66 | 2.88 | 17.20 | 0.00597 |
| (Compound 17ya − | | | | | |
| placebo) | | | | | |
| Ratio (Compound | 1.29 | 0.131 | 1.06 | 1.58 | 0.01080 |
| 17ya/placebo) | | | | | |

CI: confidence interval; SE: standard error.

TABLE 32
Study B: Mechanical Ventilation-Free Days
(Alive and Not On Mechanical Ventilation)

| | | | Lower 95% | Upper 95% |
|---|---|---|---|---|
| | Mean Days | SE | CI | CI |
| Placebo | 37.5 | 3.06 | 31.5 | 43.5 |
| Compound 17ya | 46.8 | 1.80 | 43.2 | 50.3 |

| | | | Lower | Upper | |
|---|---|---|---|---|---|
| Contrast Analysis | Estimate | SE | 95% CI | 5% CI | Value |
| Absolute difference | 9.29 | 3.55 | 2.33 | 16.30 | 0.00888 |
| (Compound 17ya - | | | | | |
| placebo) | | | | | |
| Ratio (Compound | 1.25 | 0.113 | 1.05 | 1.49 | 0.01420 |
| 17ya/placebo) | | | | | |

CI: confidence interval;
SE: standard error.

Mortality by Day 60 Considering Standard of Care Treatments

In Study B, patients were permitted to receive COVID-19 standard of care treatments including corticosteroids and remdesivir. Additional analyses were conducted to determine the mortality in patients who did or did not receive various standard of care treatments. The following tables describe the number of subjects by arm (including deaths on-study) for: Subjects receiving corticosteroids or remdesivir ≥7 days prior to randomization (Table 33); Subjects receiving corticosteroids during admission (after Day 3 on the study) (Table 34); and Subjects receiving remdesivir during admission (after Day 3 on the study) (Table 35).

Considering these data, the following conclusions are made:

No difference was observed in patients who were admitted to the hospital and received standard of care for ≥7 days prior to randomization (54.5% relative reduction in mortality) versus the patients that were in the hospital for <7 days prior to randomization or did not receive corticosteroid or remdesivir ≥7 days prior to randomization (51.1% relative reduction in mortality). Therefore, it is reasonable to conclude that Compound 17ya treatment should not be limited by the number of days in the hospital prior to initiation of therapy.

No difference was observed in patients receiving remdesivir during the study (55.9% relative reduction in mortality) vs. patients that did not receive remdesivir during the study (50.3% relative reduction in mortality). Therefore, it is reasonable to conclude that Compound 17ya may be administered with or without remdesivir coadministration.

The effectiveness of Compound 17ya in mortality benefit appears to be predominantly in patients that received concomitant corticosteroid therapy on or after Day 3 of the study (63.9% relative reduction in mortality in combination with corticosteroid therapy verses corticosteroid alone [placebo]). Therefore, it is reasonable to conclude that Compound 17ya can be co-administered with a corticosteroid.

The effectiveness of Compound 17ya in mortality benefit is observed in all subgroups of patients regardless of standard of care treatment or when the standard of care treatment was started during the study. This observation further supports the robustness of the finding that Compound 17ya has a meaningful benefit in reducing mortality in this patient population.

TABLE 33
Study B: Subjects Receiving Corticosteroids or Remdesivir
≥7 days Prior to Randomization

| | | | Absolute Change | Relative |
|---|---|---|---|---|
| | Compound | | (percentage | Change |
| | 17ya | Placebo | points) | (%) |
| NO | 112 | 60 | | |
| Deaths (%) | 21 (18.8%) | 23 (38.3%) | −19.6 | −51.1% |
| YES | 22 | 10 | | |
| Deaths (%) | 4 (18.2%) | 4 (40.0%) | −21.8 | −54.5% |

NOTE:
The mortality presented in this table is up to Day 60. The 6 subjects (4 in Compound 17ya and 2 in placebo) for whom vital status is unknown on Day 60 are considered as alive.

TABLE 34
Study B: Subjects Receiving Corticosteroids During Admission
(Co-administration On or After Day 3 of the Study)

| | | | Absolute Change | Relative |
|---|---|---|---|---|
| | Compound | | (percentage | Change |
| | 17ya | Placebo | points) | (%) |
| NO | 45 | 25 | | |
| Deaths (%) | 10 (22.20%) | 6 (24.0%) | −1.8 | −7.4% |
| YES | 89 | 45 | | |
| Deaths (%) | 15 (16.9%) | 21 (46.7%) | −29.8 | −63.9% |

NOTE:
The mortality presented in this table is up to Day 60. The 6 subjects (4 in Compound 17ya and 2 in placebo) for whom vital status is unknown on Day 60 are considered as alive.

TABLE 35
Study B: Subjects Receiving Remdesivir During Admission
(Co-administration On or After Day 3 of the Study)

| | | | Absolute Change | Relative |
|---|---|---|---|---|
| | Compound | | (percentage | Change |
| | 17ya | Placebo | points) | (%) |
| NO | 117 | 61 | | |
| Deaths (%) | 20 (17.1%) | 21 (34.4%) | −17.3 | −50.3% |
| YES | 17 | 9 | | |
| Deaths (%) | 5 (29.4%) | 6 (66.7%) | −37.3 | −55.9% |

NOTE:
The mortality presented in this table is up to Day 60. The 6 subjects (4 in Compound 17ya and 2 in placebo) for whom vital status is unknown on Day 60 are considered as alive.

Comparison of Mortality Rates Observed in Phase 3 COVID-19 Compound 17ya Study (STUDY B) with Contemporaneous COVID-19 Studies

There have been numerous other contemporary COVID-19 clinical trials with comparable baseline severities (primarily WHO 4, 5 and 6 ordinal severity) conducted in a similar timeframe to the Phase 3 COVID-19 Compound 17ya study (STUDY B) and which reported the placebo group (standard of care) mortality rates separately by disease severity. It is noted that because clinical studies are conducted under widely varying conditions including different SARS-CoV-2 variants, adverse reaction rates (including deaths) observed in the clinical studies of a drug cannot be directly compared to rates in the clinical studies of another drug and may not predict the rates observed in a broader patient population in clinical practice. Despite these limitations, the Sponsor has conducted a comparative analysis of the mortality rates observed in the placebo (standard of care) group in these contemporaneous COVID-19 studies compared to the Phase 3 COVID-19 Compound 17ya Study B (full analysis, interim analysis, and US vs. OUS; see Table 36).

In collaboration with and at the direction of the US FDA, the Phase 3 COVID-19 Compound 17ya study purposefully enrolled patients who were at the highest risk for death and patients that had already shown evidence of disease progression. Specifically, the key inclusion criteria to assure the highest risk population was enrolled were: Patients were required to have an oxygen saturation level of ≤94% on room air (prior to oxygen support); Patients requiring supplemental oxygen (WHO 4) were required to have at least one high risk comorbidity (defined by and received from the FDA); Patients requiring high-flow oxygen, non-invasive ventilation (NIV) or high-flow oxygen (WHO 5) with or without a high-risk comorbidity; and/or Patients requiring mechanical ventilation (WHO 6) with or without high-risk comorbidity.

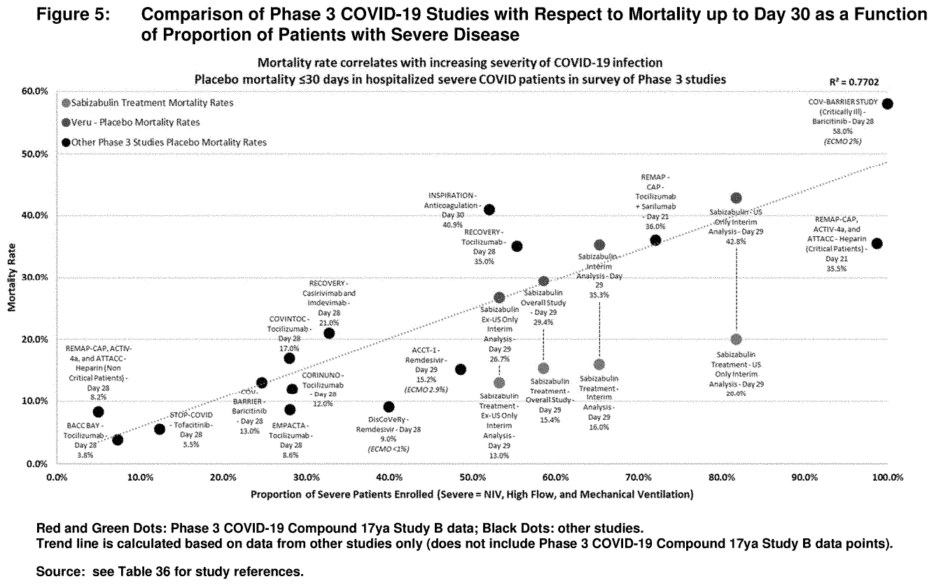

The patient populations at the greatest risk that drive the higher mortality rates are those who had an ambient air oxygen saturation level of ≤94% and who required NIV or high-flow oxygen or mechanical ventilation. The Phase 3 clinical studies referenced in the NIH COVID-19 treatment clinical guidelines were surveyed for mortality rates for patients in the placebo groups who required NIV or high-flow oxygen or mechanical ventilation at baseline and also had data for these risk categories individually reported. To visualize mortality rates across studies, the Sponsor has plotted the mortality rate observed in the control group (placebo plus standard of care) in each of the studies in Table 36 by the proportion of patients in each study with severe disease (defined as WHO 5 or 6; see FIG. 5). The regression line in FIG. 5 was calculated based on all of the contemporary COVID-19 studies (black dots) but did not include the Phase 3 COVID-19 Compound 17ya study (Study B; red and green dots). The R² of this line is 0.7702. This analysis shows that the overall mortality rate (%) of the standard of care (placebo) group of COVID-19 patients correlates to the proportion (%) of patients enrolled in the studies with severe disease at baseline (WHO 5 or 6). Additionally, when the Interim Analysis population, US-only Interim Analysis population, OUS-only Interim Analysis population, and the Full Study population from Phase 3 COVID-19 Compound 17ya study (Study B) are plotted on the graph (red dots), the placebo mortality rates in these various populations from the Compound 17ya study fit within the mortality rates observed in these contemporaneous studies. This graphical representation also illustrates the mortality benefit of Compound 17ya treatment (green dots) compared to placebo (red dots).

Based on the analysis presented above, the mortality rates in the placebo group in the Phase 3 COVID-19 Compound 17ya study (STUDY B) (Interim Analysis population, US-only Interim Analysis population, OUS-only Interim Analysis population, and the Full Study population) are consistent and expected based on the proportion of severe patients enrolled. Furthermore, one would expect the death rate in the placebo group at Day 60, the primary endpoint in the Compound 17ya Phase 3 COVID-19 Compound 17ya study (Study B), to also be even higher than the death rates from studies that reported only up to Day 30.

TABLE 36
Phase 3 COVID-19 Studies Included in the Mortality Comparison Analysis

| Trial | Citation | Reference |
|---|---|---|
| Veru | Data on File at Veru | Data on file |
| Overall Study | | |
| Veru | Barnette, et al., Oral Compound 17ya for High-Risk, Hospitalized Adults with Covid- | (Barnette et al., |
| Interim | 19: Interim Analysis, The New England Journal of Medicine Evidence, July 2022, | 2022) |
| Analysis | https://doi.org/10.1056/EVIDoa2200145 | |
| REMAP - | The REMAP-CAP Investigators, Interleukin-6 Receptor Antagonists in Critically Ill | (Gordon et al.; |
| CAP | Patients with Covid-19, The New England Journal of Medicine, April 2021, N Engl J | 2021) |
| | Med 2021; 384: 1491-1502, DOI: 10.1056/NEJMoa2100433 | |
| RECOVERY | RECOVERY Collaborative Group, Tocilizumab in patients admitted to hospital with | (Abani et al., |
| Trial | COVID-19 (RECOVERY): a randomised, controlled, open-label, platform trial, The | 2021) |
| | Lancet, May 2021, VOLUME 397, ISSUE 10285, P1637-1645, | |
| | https://doi.org/10.1016/S0140-6736(21)00676-0 | |
| EMPACTA | Salama, et al., Tocilizumab in Patients Hospitalized with Covid-19 Pneumonia, The | (Salama et al.; |
| | New England Journal of Medicine, January 2021, N Engl J Med 2021; 384: 20-30 | 2021) |
| | DOI: 10.1056/NEJMoa2030340 | |
| COVINTOC | Soin, et al., Tocilizumab plus standard care versus standard care in patients in India | (Soin et al., |
| | with moderate to severe COVID-19-associated cytokine release syndrome | 2021) |
| | (COVINTOC): an open-label, multicentre, randomised, controlled, phase 3 trial, The | |
| | Lancet, March 2021, VOLUME 9, ISSUE 5, P511-521, | |
| | https://doi.org/10.1016/S2213-2600(21)00081-3 | |
| CORIMUNO | Hermine, et al., Effect of Tocilizumab vs Usual Care in Adults Hospitalized With | (Hermine et al.; |
| | COVID-19 and Moderate or Severe Pneumonia A Randomized Clinical Trial, JAMA | 2021) |
| | Network, October 2020, JAMA Intern Med. 2021; 181(1): 32-40. | |
| | doi: 10.1001/jamainternmed.2020.6820 | |
| BACC BAY | Stone, et al., Efficacy of Tocilizumab in Patients Hospitalized with Covid-19, The | (Stone et al.; |
| | New England Journal of Medicine, December 2020, N Engl J Med 2020; 383: 2333- | 2020) |
| | 2344 DOI: 10.1056/NEJMoa2028836 | |
| COV- | Marconi, et al., Efficacy and safety of baricitinib for the treatment of hospitalised | (Marconi et al., |
| BARRIER | adults with COVID-19 (COV-BARRIER): a randomised, double-blind, parallel- | 2021) |
| (Primary) | group, placebo-controlled phase 3 trial, The Lancet, December 2021, VOLUME 9, | |
| | ISSUE 12, P1407-1418, https://doi.org/10.1016/S2213-2600(21)00331-3 | |
| STOP-COVID | Guimarães, et al., Tofacitinib in Patients Hospitalized with Covid-19 Pneumonia, The | (Guimarães et |
| | New England Journal of Medicine, July 2021, N Engl J Med 2021; 385: 406-415 | al., 2021) |
| | DOI: 10.1056/NEJMoa2101643 | |
| RECOVERY | RECOVERY Collaborative Group, Casirivimab and imdevimab in patients admitted | (Abani et al., |
| | to hospital with COVID-19 (RECOVERY): a randomised, controlled, open-label, | 2022) |
| | platform trial, The Lancet, February 2022, VOLUME 399, ISSUE 10325, P665-676, | |
| | DOI: https://doi.org/10.1016/S0140-6736(22)00163-5 | |
| ACCT-1 | Beigel, et al., Remdesivir for the Treatment of Covid-19 - Final Report, The New | (Beigel et al., |
| | England Journal of Medicine, November 2020, N Engl J Med 2020; 383: 1813-1826 | 2020) |
| | DOI: 10.1056/NEJMoa2007764 | |
| REMAP-CAP, | The REMAP-CAP, ACTIV-4a, and ATTACC Investigators, Therapeutic | (Goligher et al., |
| ACTIV-4a, | Anticoagulation with Heparin in Critically Ill Patients with Covid-19, The New | 2021) |
| and ATTACC | England Journal of Medicine, August 2021, N Engl J Med 2021; 385: 777-789 DOI: | |
| | 10.1056/NEJMoa2103417 | |
| REMAP-CAP, | The ATTACC, ACTIV-4a, and REMAP-CAP Investigators, Therapeutic | (Lawler et al., |
| ACTIV-4a, | Anticoagulation with Heparin in Noncritically Ill Patients with Covid-19, The New | 2021) |
| and ATTACC | England Journal of Medicine, August 2021, N Engl J Med 2021; 385: 790-802 DOI: | |
| | 10.1056/NEJMoa2105911 | |
| DisCoVeRy | Ader, et al., Remdesivir plus standard of care versus standard of care alone for the | (Ader et al.; |
| | treatment of patients admitted to hospital with COVID-19 (DisCoVeRy): a phase 3, | 2022) |
| | randomised, controlled, open-label trial, The Lancet, September 2021, VOLUME 22, | |
| | ISSUE 2, P209-221, DOI: https://doi.org/10.1016/S1473-3099(21)00485-0 | |
| INSPIRATION | INSPIRATION Investigators, Effect of Intermediate-Dose vs Standard-Dose | (Sadeghipour et |
| | Prophylactic Anticoagulation on Thrombotic Events, Extracorporeal Membrane | al., 2021) |
| | Oxygenation Treatment, or Mortality Among Patients With COVID-19 Admitted to | |
| | the Intensive Care Unit The INSPIRATION Randomized Clinical Trial, JAMA | |
| | Network, March 2021, JAMA. 2021; 325(16): 1620-1630. | |
| | doi: 10.1001/jama.2021.4152 | |
| COV- | Ely, et al., Efficacy and safety of baricitinib plus standard of care for the treatment of | (Ely et al.; |
| BARRIER | critically ill hospitalised adults with COVID-19 on invasive mechanical ventilation or | 2022) |
| STUDY | extracorporeal membrane oxygenation: an exploratory, randomised, placebo- | |
| (Critically Ill) | controlled trial, The Lancet, February 2022, VOLUME 10, ISSUE 4, P327-336, | |
| | DOI: https://doi.org/10.1016/S2213-2600(22)00006-6 | |

Mortality or Drug Dosing by Nasogastric Tube by Day 60 in Patients Who Started Treatment Orally

Efficacy analyses were also conducted to examine the mortality or dosing through nasogastric tube of patients who started treatment orally. In this analysis, based on Kaplan-Meier estimates, the probability of dying or receiving study drug dosing through a nasogastric tube was numerically lower for Compound 17ya 9 mg versus placebo at each assessed time point (Table 37). Treatment comparisons using log-rank and Wilcoxon χ2 tests (Compound 17ya versus placebo) were statistically significant in favor of Compound 17ya:

Log-rank χ2: 5.602, P=0.0208

Wilcoxon χ2: 5.183, P=0.0258

TABLE 37
Study B: Kaplan-Meier Estimates for Overall Mortality or Dosing Through Nasogastric Tube
(Patients who Started Treatment Orally in the Intent-to-Treat Set)

| | | | | | Absolute Risk Reduction | |
|---|---|---|---|---|---|---|
| | | | | | (Placebo versus | |
| | Compound 17ya 9 mg | | Placebo | | Compound 17ya 9 mg) | |
| | (N = 117) | | (N = 62) | | Estimate (95% CI) | |
| Number (%) of patients who died | 26 | (22.2) | 24 | (38.7) | — | |
| Number (%) of patients censored | 91 | (77.8) | 38 | (61.3) | — | |
| Number (%) of patients who died | 26 | (22.2) | 24 | (38.7) | — | |
| Number (%) of patients censored | 91 | (77.8) | 38 | (61.3) | — | |
| Kaplan-Meier Estimates | | | | | | |
| 25th percentile (95% CI) | NA | (11.0, NA) | 10.0 | (6.0, 36.0) | — | |
| Median (95% CI) | NA | (NA, NA) | NA | (39.0, NA) | — | |
| 75th percentile (95% CI) | NA | (NA, NA) | NA | (NA, NA) | — | |
| Probability of dying by Day 15 (95% CI) | 19.8 | (13.6, 28.3) | 30.7 | (20.8, 43.8) | 10.9 | (−2.7, 24.5) |
| Probability of dying by Day 22 (95% CI) | 19.8 | (13.6, 28.3) | 32.3 | (22.2, 45.5) | 12.5 | (−1.2, 26.3) |
| Probability of dying by Day 29 (95% CI) | 20.6 | (14.3, 29.2) | 32.3 | (22.2, 45.5) | 11.7 | (−2.1, 25.5) |
| Probability of dying by Day 45 (95% CI) | 22.4 | (15.8, 31.1) | 39.1 | (28.2, 52.5) | 16.7 | (2.3, 31.1) |
| Probability of dying by Day 60 (95% CI) | 22.4 | (15.8, 31.1) | 39.1 | (28.2, 52.5) | 16.7 | (2.3, 31.1) |

Abbreviations: CI = confidence interval;
NA = not applicable.

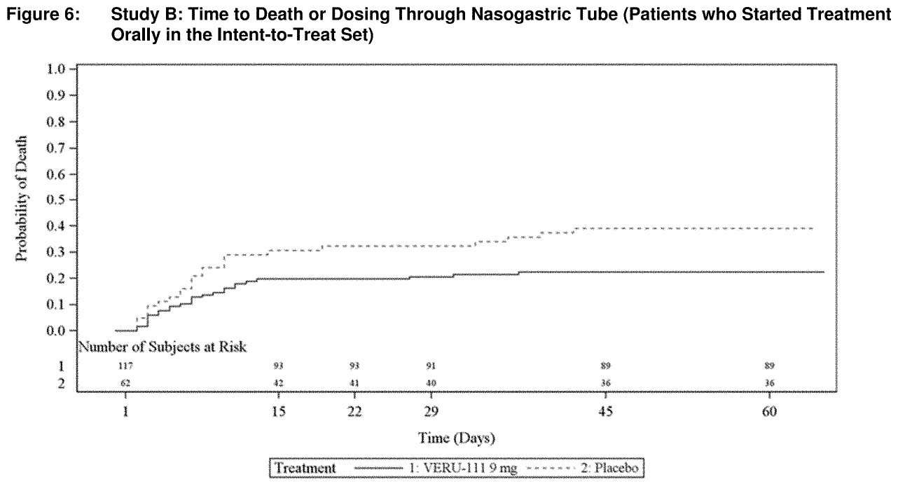

There was also clear separation between the Compound 17ya 9 mg and placebo Kaplan-Meier curves for time to death or dosing through nasogastric tube (FIG. 6).

Efficacy Conclusions

Overall, data from Phase 2 (Study A) and Phase 3 (Study B) clinical studies demonstrate the efficacy of Compound 17ya in hospitalized adult patients with moderate to severe COVID-19 infection who were at high risk for ARDS.

Data from Phase 2 (Study A) Demonstrate the Following:

Compound 17ya shows clinically meaningful outcomes in this proof-of-concept Phase 2 study.

Results for the primary efficacy endpoint (proportion of subjects alive without respiratory failure) support that Compound 17ya is efficacious for the treatment of COVID-19 in patients at high risk for ARDS at Day 15, Day 22, and Day 29.

All the parameters measured in the study (including proportion of patients alive without respiratory failure, mortality status up to Day 60, days in ICU, and days on mechanical ventilation) showed clinically meaningful outcomes with Compound 17ya compared to placebo and there were no parameters that did not indicate benefit with Compound 17ya treatment compared to placebo (although some parameters did not reach statistical significance in this small study).

Data from Phase 3 (Study B) Demonstrate the Following:

Interim Analysis data from Study B (First 150 patients enrolled) show that treatment with Compound 17ya 9 mg once daily resulted in a clinically meaningful and statistically significant 55.2% relative reduction in deaths (p=0.0042) at Day 60; findings from this interim analysis were subsequently peer-reviewed and published in the New England Journal of Medicine Evidence (Barnette et al., 2022). Based on the interim analysis, the study was stopped by the Independent Data Monitoring Committee due to clear evidence of efficacy.

Data from the complete dataset in the Study B study (ITT population; 204 patients) show clinically meaningful and statistically significant reductions in mortality compared to placebo at Day 60 (primary endpoint), Day 29, and Day 15. Specifically, treatment with Compound 17ya resulted in a clinically relevant and statistically significant 51.6% relative reduction (20.5% absolute reduction) in mortality up to Day 60 compared to placebo (p=0.0046).

Prespecified subgroup analyses relating to standard of care treatment indicate that Compound 17ya treatment is effective when administered both as first-line therapy or as part of first-line therapy (co-administered with corticosteroid therapy) in hospitalized patients requiring oxygen support, and that Compound 17ya treatment results in a clinically meaningful reduction in mortality in patients regardless of prior initiation of remdesivir treatment.

Clinically meaningful reductions in mortality were also observed in patients regardless of vaccination status or vaccination type.

Other prespecified subgroup analyses show that Compound 17ya treatment results in a clinically meaningful reduction in mortality in each of the WHO Ordinal Scores at baseline (WHO 4, WHO 5, and WHO 6), and also in all of the countries in which a sufficient number of patients were enrolled (US, Brazil, and Bulgaria).

It is noted that the overall mortality in the placebo group in the US (56.5%) is higher than the OUS population (31.1%), which is most likely related to the difference in the proportion of patients with severe COVID-19 infection at baseline between the US (81.7% of the patients were WHO 5 or 6) and OUS (44.4% of the patients were WHO 5 or 6). Regardless of region, the reduction in mortality in the Compound 17ya group is marked compared to the placebo group.

Sensitivity analyses were conducted to test for robustness of the study data and found that mortality reductions with Compound 17ya treatment are observed in all subgroups concerning demographics, baseline characteristics, SARS-CoV-2 variant, comorbidities, vaccination status, and COVID-19 standard of care.

A backward logistic regression was also performed on the primary efficacy endpoint data, taking into account multiple covariates including treatment, region, sex, remdesivir use at baseline, dexamethasone use at baseline, WHO strata, selected respiratory issues, asthma, history of heart failure, diabetes, severe obesity, age ≥65 years, and ≥3 of the following: selected respiratory issues, history of heart failure, diabetes, severe obesity, age ≥65. The results from this analysis showed that treatment with Compound 17ya continued to show a highly statistically significant effect on reducing mortality by Day 60 compared to placebo (p=0.0050). It is concluded from this analysis that any small potential imbalances in these study factors do not appear to have a singular or combined effect on the observed benefit of Compound 17ya in the primary study endpoint, reduction in death by Day 60.

Analysis of Secondary Endpoints in Study B Further Support the Efficacy of Compound 17ya and Show that:

Compared to placebo, Compound 17ya treatment resulted in a 40.6% relative reduction in mortality or respiratory failure at Day 29 (p=0.0186), and a 49.6% relative reduction at Day 60 (p=0.0066).

A 39.2% relative reduction in days in the ICU (p=0.0045), and a 44.3% relative reduction in days on mechanical ventilation (p=0.0038) were observed in the Compound 17ya group compared to placebo by the protocol defined and FDA required analysis.

Compound 17ya treatment also resulted in a statistically significant (p=0.0463) reduction in days in the hospital by the protocol defined and FDA required analysis.

Secondary endpoint analysis of mean viral load from baseline to last on-study assessment showed a 42.9% relative reduction in the Compound 17ya treated group compared to a 412% increase in the placebo group.

Sensitivity analyses to test for robustness of data in the secondary endpoints showed statistically significant and clinically meaningful increases in the Compound 17ya group compared to placebo for mean days alive and out of the hospital, mean days alive and not in the ICU, and mean days alive and not on mechanical ventilation. This analysis further confirms the benefit of Compound 17ya in hospitalized patients with moderate to severe COVID-19 at high risk for ARDS.

Additional Efficacy Analyses for Study B Requested by FDA During the EUA Review Process Further Indicate that:

Compound 17ya treatment is effective when co-administered with a corticosteroid therapy, and that the efficacy of Compound 17ya compared to placebo is unaffected by the number of days patients are hospitalized prior to receiving Compound 17ya, or by treatment with remdesivir.

It is noted that when comparing the placebo mortality rate observed in Study B to placebo (standard of care) groups in contemporaneous COVID-19 studies, the mortality rates in the placebo group for the interim and full analyses of Study B (as well as OUS and US-only subgroups) are consistent and as expected based on the proportion of severe COVID-19 patients enrolled in the study.

Compound 17ya treatment shows a statistically significant reduction in ‘mortality or progression to dosing through nasogastric tube’ by Day 60 compared to placebo.

Overall, when the data from the Phase 2 and Phase 3 COVID-19 Compound 17ya studies are combined, the Day 60 mortality rate for patients treated with Compound 17ya was 17.4% (26/149) compared to 37.5% (33/88) in placebo patients representing a 20.1% absolute reduction and 53.6% relative reduction in mortality at Day 60 in the Compound 17ya treated patients compared to placebo.

Overview of Safety

Safety

Compound 17ya has been investigated in two double-blind, placebo-controlled clinical trials in moderate to severe COVID-19 patients who were at high risk for ARDS (Phase 2 Study A and Phase 3 Study B). In these studies, Compound 17ya was administered to hospitalized patients once per day orally or via nasogastric tube for up to 21 days or hospital discharge (whichever came first) at a dose of 18 mg (powder in capsule [PIC] formulation, Phase 2 study), or 9 mg (formulated capsule [FC] formulation, Phase 3 study).

Extent of Exposure

Study A

A total of 39 subjects received at least 1 dose of study drug (19 subjects in the 18 mg PIC Compound 17ya group and 20 subjects in the placebo group). The mean (SD) treatment exposure was comparable in both the treatment groups: 9.0 days (6.64) in the Compound 17ya group and 11.2 days (6.74) in the placebo group.

Study B

In Study B a total of 199 subjects received at least 1 dose of study drug (130 subjects in the 9 mg Compound 17ya treated group and 69 subjects in the placebo group). The mean (SD) treatment exposure was comparable in both the treatment groups: 11.4 days (6.56) in the Compound 17ya group and 11.6 days (6.01) in the placebo group.

Treatment-Emergent Adverse Events

In Study A, 24 subjects (61.5%) reported a total of 72 TEAEs. TEAEs were reported by 12 subjects (63.2%) who received Compound 17ya and 12 subjects (60.0%) who received placebo. Overall, the most commonly (≥20%) reported SOC categories for TEAEs were gastrointestinal disorders (23.1%) and investigations (20.5%).

In the Compound 17ya group, the most commonly (≥2 subjects) reported TEAEs by preferred term (PT) were constipation and aspartate aminotransferase increased (3 subjects each) and alanine aminotransferase increased (2 subjects). All other TEAEs were singular events, and none of these events were deemed to be related to study drug.

In the placebo group, the most commonly (≥2 subjects) reported TEAEs by PT were respiratory failure (4 subjects), pneumothorax and septic shock (3 subjects each), and acute kidney injury, alanine aminotransferase increased, constipation, and pneumomediastinum (2 subjects each). All other TEAEs were singular events, and none of these events were deemed to be related to study drug.

In Study A, 1 subject in the Compound 17ya group discontinued due to an AE. This subject, who received two doses of Compound 17ya, developed a Grade 4 event of cardiac arrest and was discontinued on the same day. The subject had SpO2 of 84 at baseline, was non-compliant with study procedures, and refused forced O2. The subject's O2 level continued to drop and dropped to 55 at the time of cardiac arrest. The subject did not recover from the event and did not complete the study.

Study B

Overall, 136 subjects (68.3%) reported a total of 663 TEAEs (Error! Reference source not found). TEAEs were reported by 82 subjects (63.1%) who received Compound 17ya and 54 subjects (78.3%) who received placebo. Overall, the most commonly (≥20% in either treatment group) reported System Organ Class (SOC) categories for TEAEs were: (1) cardiac disorder (12.3% Compound 17ya vs. 30.4% placebo); (2) infections and infestations (30.0% Compound 17ya vs. 40.6% placebo); (3) metabolism and nutrition disorders (16.2% Compound 17ya vs. 26.1% placebo); (4) respiratory, thoracic, and mediastinal disorders (25.4% Compound 17ya vs. 46.4% placebo), and (5) vascular disorders (13.8% Compound 17ya vs. 24.6% placebo). Additionally, a review of the System Organ Classes that had a higher incidence of events in the Compound 17ya group compared to placebo was conducted. These categories are listed in Table 38 below.

TABLE 38
Study B: System Organ Class Categories
in Which a Higher Proportion of Patients Reported TEAEs
in the Compound 17ya Group Compared to Placebo

| | Compound 17ya | Placebo |
|---|---|---|
| System Organ Class | (N = 130) | (N = 69) |
| Blood and lymphatic system disorders | 12 (9.2) | 4 (5.8) |
| Gastrointestinal disorders | 21 (16.2) | 6 (8.7) |
| Skin and subcutaneous tissue disorders | 10 (7.7) | 2 (2.9) |

In the Compound 17ya group, the most commonly (≥5% subjects) reported TEAEs by PT were: anemia (5.4% Compound 17ya vs. 4.3% placebo); constipation (6.9% Compound 17ya vs. 8.7% placebo); pneumonia (6.2% Compound 17ya vs. 13.0% placebo); urinary tract infection (6.2% Compound 17ya vs. 1.4% placebo); acute kidney injury (8.5% Compound 17ya vs. 11.6% placebo); acute respiratory failure (5.4% Compound 17ya vs. 4.3% placebo); and respiratory failure (10.0% Compound 17ya vs. 20.3% placebo).

Considering the TEAEs and SOCs reported in Study B, the following observations and conclusions are made:

The benefit: risk ratio of Compound 17ya is clinically relevant based on the reduction in deaths (51.6% reduction in Compound 17ya group) and reduction in life threatening TEAEs in the Compound 17ya group compared to placebo such as: septic shock (79.2% reduction), pneumonia (52.3% reduction), pneumothorax (92.1% reduction), and respiratory failure (50.7% reduction).

The proportion of patients in the Compound 17ya group that report any TEAE (19.4% fewer) and any serious TEAE (37.1% fewer) was lower than in the placebo group.

The efficacy of Compound 17ya in the treatment of COVID-19 is further demonstrated in the reduction in some TEAEs often associated with the progression of COVID-19 infection (septic shock, acute kidney injury, hypoxia, pneumothorax, respiratory failure, and hypotension).

The SOCs and TEAEs that are observed at a higher rate in the Compound 17ya group compared to the placebo group (SOCs: Blood and lymphatic system disorders, Gastrointestinal disorders, Skin and subcutaneous tissue disorders; TEAEs: anemia, diarrhea, vomiting, urinary tract infections, various skin disorders such as allergic dermatitis, intertrigo, rash, and decubitus ulcer) can be managed with therapy in hospitalized patients being treated with Compound 17ya and are not (or are not immediately) life threatening.

The SOCs in which the incidence of TEAEs are at least 20% lower in the Compound 17ya group compared to the placebo group are: (a) Cardiac Disorders (−59.5%); (b) Infections and infestations (−26.1%); (c) Metabolism and nutrition disorders (−37.9%); (d) Musculoskeletal and connective tissue disorders (−47.2%); (e) Nervous system disorders (−40.8%); (f) Psychiatric disorders (−20.7%); (g) Renal and urinary disorders (−38.8%); (h) Respiratory, thoracic, and mediastinal disorders (−45.3%); and (i) Vascular disorders (−43.9%).

TABLE 39
Study B: Treatment-Emergent Adverse Events Occurring in
≥5% of Patients in Any Treatment Group (Safety Set)

| | Compound | | | | | |
|---|---|---|---|---|---|---|
| | 17ya 9 mg | | Placebo | | Overall | |
| System Organ Class | (N = 130) | | (N = 69) | | (N = 199) | |
| Preferred Term | n (%) | | | | | |
| Subjects with any TEAE | 82 | (63.1) | 54 | (78.3) | 136 | (68.3) |
| Blood and lymphatic | 12 | (9.2) | 4 | (5.8) | 16 | (8.0) |
| system disorders | | | | | | |
| Anemia | 7 | (5.4) | 3 | (4.3) | 10 | (5.0) |
| Cardiac disorders | 16 | (12.3) | 21 | (30.4) | 37 | (18.6) |
| Atrial fibrillation | 6 | (4.6) | 5 | (7.2) | 11 | (5.5) |
| Bradycardia | 6 | (4.6) | 5 | (7.2) | 11 | (5.5) |
| Gastrointestinal disorders | 21 | (16.2) | 6 | (8.7) | 27 | (13.6) |
| Constipation | 9 | (6.9) | 6 | (8.7) | 15 | (7.5) |
| General disorders and | 9 | (6.9) | 4 | (5.8) | 13 | (6.5) |
| administration site | | | | | | |
| conditions | | | | | | |
| Infections and infestations | 39 | (30.0) | 28 | (40.6) | 67 | (33.7) |
| Pneumonia | 8 | (6.2) | 9 | (13.0) | 17 | (8.5) |
| Septic shock | 2 | (1.5) | 5 | (7.2) | 7 | (3.5) |
| Urinary tract infection | 8 | (6.2) | 1 | (1.4) | 9 | (4.5) |
| Investigations | 20 | (15.4) | 10 | (14.5) | 30 | (15.1) |
| Metabolism and nutrition | 21 | (16.2) | 18 | (26.1) | 39 | (19.6) |
| disorders | | | | | | |
| Hyperkalemia | 6 | (4.6) | 6 | (8.7) | 12 | (6.0) |
| Hypernatremia | 6 | (4.6) | 4 | (5.8) | 10 | (5.0) |
| Hypokalemia | 6 | (4.6) | 5 | (7.2) | 11 | (5.5) |
| Hypophosphatemia | 2 | (1.5) | 4 | (5.8) | 6 | (3.0) |
| Musculoskeletal and | 5 | (3.8) | 5 | (7.2) | 10 | (5.0) |
| connective tissue | | | | | | |
| disorders | | | | | | |
| Nervous system disorders | 10 | (7.7) | 9 | (13.0) | 19 | (9.5) |
| Psychiatric disorders | 12 | (9.2) | 8 | (11.6) | 20 | (10.1) |
| Anxiety | 4 | (3.1) | 4 | (5.8) | 8 | (4.0) |
| Delirium | 5 | (3.8) | 4 | (5.8) | 9 | (4.5) |
| Renal and urinary disorders | 15 | (11.5) | 13 | (18.8) | 28 | (14.1) |
| Acute kidney injury | 11 | (8.5) | 8 | (11.6) | 19 | (9.5) |
| Respiratory, thoracic and | 33 | (25.4) | 32 | (46.4) | 65 | (32.7) |
| mediastinal disorders | | | | | | |
| Acute respiratory failure | 7 | (5.4) | 3 | (4.3) | 10 | (5.0) |
| Hypoxia | 3 | (2.3) | 4 | (5.8) | 7 | (3.5) |
| Pneumothorax | 1 | (0.8) | 7 | (10.1) | 8 | (4.0) |
| Pulmonary embolism | 4 | (3.1) | 3 | (4.3) | 7 | (3.5) |
| Respiratory failure | 13 | (10.0) | 14 | (20.3) | 27 | (13.6) |
| Skin and subcutaneous | 10 | (7.7) | 2 | (2.9) | 12 | (6.0) |
| tissue disorders | | | | | | |
| Vascular disorders | 18 | (13.8) | 17 | (24.6) | 35 | (17.6) |
| Hypotension | 5 | (3.8) | 8 | (11.6) | 13 | (6.5) |

AEs are coded using MedDRA version 24.0.
Abbreviations: AE = adverse event;
MedDRA = Medical Dictionary for Regulatory Activities;
TEAE = treatment-emergent adverse event.

COVID-19 Studies: Phase 2 and Phase 3 Combined Analysis of TEAEs

The most common TEAEs (≥2%) in the Compound 17ya treated group in the Phase 2 and Phase 3 clinical studies combined are presented in Table 40. Overall, the display of TEAEs in this combined safety population is similar to that observed in the Phase 3 Study B, which is expected as the Phase 3 study was much larger than the Phase 2 study.

TABLE 40
Studies A and B Combined Analysis:
TEAEs Occurring in ≥2% of Subjects Receiving Compound 17ya by PT

| | Compound 17ya 9 mg | Placebo |
|---|---|---|
| Adverse Reaction | (N = 149) | (N = 89) |
| Anemia | 4.7% | 4.5% |
| Atrial Fibrillation | 4.0% | 6.7% |
| Bradycardia | 4.0% | 5.6% |
| Tachycardia | 2.0% | 1.1% |
| Constipation | 8.1% | 9.0% |
| Diarrhea | 3.4% | 1.1% |
| Dyspepsia | 2.0% | 0 |
| Vomiting | 2.0% | 0 |
| Pyrexia | 3.4% | 0 |
| COVID-19 | 3.4% | 4.5% |
| Infection | 2.0% | 0 |
| Pneumonia | 5.4% | 10.1% |
| Pulmonary sepsis | 2.0% | 1.1% |
| Sepsis | 4.7% | 4.5% |
| Septic shock | 2.0% | 9.0% |
| Urinary Tract Infection | 5.4% | 1.1% |
| Alanine aminotransferase increased | 4.0% | 4.5% |
| Aspartate aminotransferase increased | 2.7% | 2.2% |
| Fibrin D dimer increased | 2.7% | 2.2% |
| Gamma-glutamyltransferase increased | 2.7% | 2.2% |
| Serum ferritin increased | 2.0% | 2.2% |
| Transaminases increased | 4.0% | 2.2% |
| Hyperkalemia | 4.0% | 6.7% |
| Hypernatremia | 4.0% | 4.5% |
| Hypokalemia | 4.7% | 5.6% |
| Hypertension | 2.0% | 1.1% |
| Anxiety | 2.7% | 4.5% |
| Delirium | 3.4% | 4.5% |
| Acute Kidney Injury | 7.4% | 11.2% |
| Haematuria | 2.0% | 2.2% |
| Acute Respiratory Failure | 4.7% | 3.4% |
| Hypoxia | 2.0% | 4.5% |
| Pulmonary Embolism | 2.7% | 3.4% |
| Respiratory failure | 8.7% | 20.2% |
| Decubitis ulcer | 2.7% | 0 |
| Deep Vein Thrombosis | 2.0% | 1.1% |
| Hypotension | 3.4% | 10.1% |

Adverse Drug Reactions

Study A

In Study A there were no treatment-related adverse events.

Study B

There were 21 (10.6%) subjects that reported a treatment related TEAE: 13 (10.0%) subjects in the Compound 17ya group and 8 (11.6%) of the subjects in the placebo group. Gastrointestinal disorders (6 patients, 4.6%) and Investigations (5 patients, 3.8%) were the most common SOCs for treatment-related TEAEs in the Compound 17ya group, compared to Investigations (4 patients, 5.8%) and Respiratory, thoracic, and mediastinal disorders (3 patients, 4.3%) in the placebo group.

The only treatment-related TEAE reported by ≥2% of subjects in the Compound 17ya treated group was ‘Transaminases increased’ (3 patients, 2.3%). By comparison, in the placebo group, there were 2 treatment-related TEAEs reported by ≥2% of subjects: ‘Hepatic enzyme increased’ (2 patients, 2.9%), and ‘Respiratory failure’ (2 patients, 2.9%).

Deaths and Other Serious Adverse Events

Study A

Overall, 7 subjects died during the study (1 subject who received Compound 17ya and 6 subjects who received placebo). One subject who received Compound 17ya reported Grade 5 toxicity (fatal) TEAE of septic shock. Six subjects who received placebo reported Grade 5 toxicity (fatal) TEAEs of septic shock (2 subjects), respiratory failure (2 subjects), COVID-19 (1 subject), and death of unknown cause (1 subject; this subject's death occurred >7 days after treatment end and therefore was not considered treatment emergent).

A total of 8 (20.5%) subjects (3 subjects who received Compound 17ya and 5 subjects who received placebo) had serious TEAEs.

Overall, 1 subject in the study discontinued due to an AE. This subject, who received Compound 17ya, developed a Grade 4 event of cardiac arrest and was discontinued on the same day. The subject had SpO₂ of 84 at baseline, was non-compliant with study procedures, and refused forced O₂. The subject's O₂ level continued to drop and dropped to 55 at the time of cardiac arrest. The subject did not recover from the event and did not complete the study.

Study B

In patients who received at least one dose of study drug (safety set), there were 23 deaths (17.7%) in the Compound 17ya 9 mg FC group, and 25 deaths (36.2%) in the placebo group. The most common fatal TEAEs, by SOC, were infections and infestations (10 [7.7%] patients in the Compound 17ya 9 mg group and 7 [10.1%] patients in the placebo group) and respiratory, thoracic, and mediastinal disorders (8 [6.2%] patients in the Compound 17ya 9 mg group and 10 [14.5%] patients in the placebo group).

The most common fatal TEAE, by PT, was respiratory failure in both groups (5 [3.8%] patients in the Compound 17ya 9 mg group and 4 [5.8%] patients in the placebo group). The next most common fatal TEAEs reported in patients who received Compound 17ya were COVID-19 (3 [2.3%] patients; 2 [2.9%] patients in the placebo group), acute respiratory failure (2 [1.5%] patients; 3 [4.3%] patients who received placebo), and severe acute respiratory syndrome (2 [1.5%] patients; no patients in the placebo group). In patients who received placebo, the next most common fatal TEAE, by PT, was pneumonia (3 [4.3%] patients; 1 [0.8%] patient in the Compound 17ya 9 mg group). See Table 41.

TABLE 41
Study B: Fatal Treatment-Emergent Adverse Events
by System Organ Class and Preferred Term (Safety Set)

| | Compound | | |
|---|---|---|---|
| | 17ya | | |
| | 9 mg | Placebo | Overall |
| | (N = 130) | (N = 69) | (N = 199) |
| | n (%) | | |
| Number of deaths | 23 (17.7) | 25 (36.2) | 48 (24.1) |
| Cardiac disorders | 1 (0.8) | 4 (5.8) | 5 (2.5) |
| Bradycardia | 0 | 1 (1.4) | 1 (0.5) |
| Cardiac arrest | 0 | 1 (1.4) | 1 (0.5) |
| Cardio-respiratory arrest | 1 (0.8) | 1 (1.4) | 2 (1.0) |
| Cardiovascular insufficiency | 0 | 1 (1.4) | 1 (0.5) |
| General disorders and | 1 (0.8) | 2 (2.9) | 3 (1.5) |
| administration site | | | |
| conditions | | | |
| Death | 1 (0.8) | 0 | 1 (0.5) |
| Multiple organ dysfunction | 0 | 2 (2.9) | 2 (1.0) |
| syndrome | | | |
| Infections and infestations | 10 (7.7) | 7 (10.1) | 17 (8.5) |
| Burkholderia cepacia | 1 (0.8) | 0 | 1 (0.5) |
| complex infection | | | |
| COVID-19 | 3 (2.3) | 2 (2.9) | 5 (2.5) |
| Device related infection | 1 (0.8) | 0 | 1 (0.5) |
| Pneumonia | 1 (0.8) | 3 (4.3) | 4 (2.0) |
| Sepsis | 1 (0.8) | 0 | 1 (0.5) |
| Septic shock | 1 (0.8) | 2 (2.9) | 3 (1.5) |
| Severe acute respiratory | 2 (1.5) | 0 | 2 (1.0) |
| syndrome | | | |
| Nervous system disorders | 1 (0.8) | 1 (1.4) | 2 (1.0) |
| Cerebrovascular accident | 0 | 1 (1.4) | 1 (0.5) |
| Coma | 1 (0.8) | 0 | 1 (0.5) |
| Renal and urinary disorders | 1 (0.8) | 0 | 1 (0.5) |
| Renal failure | 1 (0.8) | 0 | 1 (0.5) |
| Respiratory, thoracic and | 8 (6.2) | 10 (14.5) | 18 (9.0) |
| mediastinal disorders | | | |
| Acute respiratory failure | 2 (1.5) | 3 (4.3) | 5 (2.5) |
| Hypoxia | 1 (0.8) | 2 (2.9) | 3 (1.5) |
| Pulmonary embolism | 0 | 1 (1.4) | 1 (0.5) |
| Respiratory failure | 5 (3.8) | 4 (5.8) | 9 (4.5) |
| Vascular disorders | 1 (0.8) | 1 (1.4) | 2 (1.0) |
| Hypovolemic shock | 0 | 1 (1.4) | 1 (0.5) |
| Shock | 1 (0.8) | 0 | 1 (0.5) |

AEs are coded using MedDRA version 24.0.
Abbreviations: AE = adverse event;
COVID-19 = coronavirus disease 2019;
MedDRA = Medical Dictionary for Regulatory Activities;
TEAE = treatment-emergent adverse event.

Serious TEAEs

Overall, 70 subjects (35.2%) reported serious TEAEs. Serious TEAEs were reported by 38 subjects (29.2%) who received Compound 17ya and 32 subjects (46.4%) who received placebo. The most common serious TEAEs, by SOC, were respiratory, thoracic, and mediastinal disorders (23 [17.7%] patients in the Compound 17ya 9 mg group and 23 [33.3%] patients in the placebo group) and infections and infestations (20 [15.4%] patients in the Compound 17ya 9 mg group and 15 [21.7%] patients in the placebo group).

The most common serious TEAEs, by PT, were respiratory failure (13 [10.0%] patients), acute kidney injury (6 [4.6%] patients) and acute respiratory failure (5 [3.8%] patients) in the Compound 17ya 9 mg group, and respiratory failure (14 [20.3%] patients), acute kidney injury (6 [8.7%] patients), and pneumothorax (6 [8.7%] patients) in the placebo group. See Table 42.

TABLE 42
Study B: Serious Treatment-Emergent Adverse Events
Occurring in ≥5% of Patients in Any Treatment Group,
by System Organ Class and Preferred Term (Safety Set)

| | Compound | | |
|---|---|---|---|
| | 17ya | | |
| | 9 mg | Placebo | Overall |
| | (N = 130) | (N = 69) | (N = 199) |
| | n (%) | | |
| Any TEAE | 38 (29.2) | 32 (46.4) | 70 (35.2) |
| Cardiac disorders | 3 (2.3) | 7 (10.1) | 10 (5.0) |
| Atrial fibrillation | 0 | 1 (1.4) | 1 (0.5) |
| Bradycardia | 1 (0.8) | 1 (1.4) | 2 (1.0) |
| Cardiac arrest | 0 | 3 (4.3) | 3 (1.5) |
| Cardio-respiratory arrest | 2 (1.5) | 1 (1.4) | 3 (1.5) |
| Cardiovascular insufficiency | 0 | 1 (1.4) | 1 (0.5) |
| Pulmonary valve incompetence | 0 | 1 (1.4) | 1 (0.5) |
| Infections and infestations | 20 (15.4) | 15 (21.7) | 35 (17.6) |
| Acinetobacter infection | 1 (0.8) | 0 | 1 (0.5) |
| Burkholderia cepacia | 1 (0.8) | 0 | 1 (0.5) |
| complex infection | | | |
| COVID-19 | 4 (3.1) | 3 (4.3) | 7 (3.5) |
| COVID-19 pneumonia | 0 | 1 (1.4) | 1 (0.5) |
| Clostridium difficile colitis | 1 (0.8) | 0 | 1 (0.5) |
| Device related infection | 1 (0.8) | 0 | 1 (0.5) |
| Endocarditis staphylococcal | 1 (0.8) | 0 | 1 (0.5) |
| Enterococcal sepsis | 1 (0.8) | 0 | 1 (0.5) |
| Infection | 1 (0.8) | 0 | 1 (0.5) |
| Pneumonia | 4 (3.1) | 4 (5.8) | 8 (4.0) |
| Pneumonia Acinetobacter | 1 (0.8) | 0 | 1 (0.5) |
| Pneumonia bacterial | 0 | 2 (2.9) | 2 (1.0) |
| Pulmonary sepsis | 2 (1.5) | 1 (1.4) | 3 (1.5) |
| Sepsis | 4 (3.1) | 2 (2.9) | 6 (3.0) |
| Septic shock | 2 (1.5) | 5 (7.2) | 7 (3.5) |
| Severe acute respiratory | 2 (1.5) | 0 | 2 (1.0) |
| syndrome | | | |
| Urinary tract infection | 2 (1.5) | 0 | 2 (1.0) |
| Urinary tract infection bacterial | 2 (1.5) | 0 | 2 (1.0) |
| Urosepsis | 1 (0.8) | 0 | 1 (0.5) |
| Renal and urinary disorders | 7 (5.4) | 8 (11.6) | 15 (7.5) |
| Acute kidney injury | 6 (4.6) | 6 (8.7) | 12 (6.0) |
| Renal failure | 1 (0.8) | 0 | 1 (0.5) |
| Renal impairment | 0 | 1 (1.4) | 1 (0.5) |
| Tubulointerstitial nephritis | 0 | 1 (1.4) | 1 (0.5) |
| Respiratory, thoracic and | 23 (17.7) | 23 (33.3) | 46 (23.1) |
| mediastinal disorders | | | |
| Acute respiratory failure | 5 (3.8) | 3 (4.3) | 8 (4.0) |
| Dyspnea | 1 (0.8) | 1 (1.4) | 2 (1.0) |
| Hypoxia | 2 (1.5) | 3 (4.3) | 5 (2.5) |
| Laryngeal stenosis | 1 (0.8) | 0 | 1 (0.5) |
| Organizing pneumonia | 0 | 1 (1.4) | 1 (0.5) |
| Pneumomediastinum | 0 | 1 (1.4) | 1 (0.5) |
| Pneumothorax | 1 (0.8) | 6 (8.7) | 7 (3.5) |
| Pulmonary embolism | 3 (2.3) | 3 (4.3) | 6 (3.0) |
| Pulmonary hemorrhage | 0 | 1 (1.4) | 1 (0.5) |
| Respiration abnormal | 0 | 1 (1.4) | 1 (0.5) |
| Respiratory acidosis | 0 | 1 (1.4) | 1 (0.5) |
| Respiratory failure 24.0. | 13 (10.0) | 14 (20.3) | 27 (13.6) |

AEs are coded using MedDRA version 24.0.
Abbreviations: AE = adverse event;
COVID-19 = coronavirus disease 2019;
MedDRA = Medical Dictionary for Regulatory Activities;
TEAE = treatment-emergent adverse event.

TEAEs Leading to Study Drug Discontinuation

TEAEs leading to study drug discontinuation were reported in 9 (4.5%) patients overall, including 6 (4.6%) patients in the Compound 17ya 9 mg group and 3 (4.3%) patients in the placebo group. The most commonly reported TEAEs leading to study drug discontinuation, by SOC, were investigations (2 [1.5%] patients in the Compound 17ya 9 mg group and 2 [2.9%] patients in the placebo group). As shown below in Table 43, all TEAEs leading to study drug discontinuation, by PT, were each reported in 1 patient.

Overall, TEAEs leading to discontinuation were equal between the treatment groups (4.6% in the Compound 17ya group vs. 4.3% in the placebo group). The preferred terms for the AEs that led to discontinuation in the Compound 17ya group do not represent TEAEs that are observed at a higher rate in the Compound 17ya group (COVID-19 [n=1], endocarditis staphylococcal [n=1], alanine aminotransferase increased [n=1], liver function test increased [n=1], acute kidney injury [n=1], respiratory failure [n=1]), with the exception of dysphagia (n=1) which was not observed in any patients in the placebo group.

TABLE 43
Study B: Treatment-Emergent Adverse Events Leading to
Treatment Discontinuation (Drug Withdrawn),
by System Organ Class and Preferred Term (Safety Set)

| | Compound | | |
|---|---|---|---|
| | 17ya | | |
| | 9 mg | Placebo | Overall |
| | (N = 130) | (N = 69) | (N = 199) |
| | n (%) | | |
| Any TEAE resulting in study | 6 (4.6) | 3 (4.3) | 9 (4.5) |
| drug discontinuation | | | |
| Gastrointestinal disorders | 1 (0.8) | 0 | 1 (0.5) |
| Dysphagia | 1 (0.8) | 0 | 1 (0.5) |
| Infections and infestations | 1 (0.8) | 0 | 1 (0.5) |
| COVID-19 | 1 (0.8) | 0 | 1 (0.5) |
| Endocarditis staphylococcal | 1 (0.8) | 0 | 1 (0.5) |
| Investigations | 2 (1.5) | 2 (2.9) | 4 (2.0) |
| Alanine aminotransferase | 1 (0.8) | 0 | 1 (0.5) |
| increased | | | |
| Hepatic enzyme increased | 0 | 1 (1.4) | 1 (0.5) |
| Liver function test abnormal | 0 | 1 (1.4) | 1 (0.5) |
| Liver function test increased | 1 (0.8) | 0 | 1 (0.5) |
| Renal and urinary disorders | 1 (0.8) | 0 | 1 (0.5) |
| Acute kidney injury | 1 (0.8) | 0 | 1 (0.5) |
| Respiratory, thoracic, and | 1 (0.8) | 1 (1.4) | 2 (1.0) |
| mediastinal disorders | | | |
| Dyspnea | 0 | 1 (1.4) | 1 (0.5) |
| Respiratory failure | 1 (0.8) | 0 | 1 (0.5) |

AEs are coded using MedDRA version 24.0.
Abbreviations: AE = adverse event;
COVID-19 = coronavirus disease 2019;
MedDRA = Medical Dictionary for Regulatory Activities;
TEAE = treatment-emergent adverse event.

COVID-19 Studies: Phase 2 and Phase 3 Combined Analysis of Serious TEAEs

Serious TEAEs occurred in (27.5%) of the subjects receiving Compound 17ya and (41.6%) receiving placebo in the Phase 2 and Phase 3 clinical studies combined; most serious TEAEs were COVID-19 related. The most common serious TEAEs in the Compound 17ya group observed in the Phase 2 and Phase 3 clinical trials combined are presented in Table 44. In the Compound 17ya group, the most commonly (≥5% subjects) reported serious TEAE by PT was respiratory failure (8.7% Compound 17ya vs. 18.0% placebo)

TABLE 44
Studies A and B Combined Analysis:
Serious TEAEs Reported by ≥2% of Patients Treated with
Compound 17ya by PT

| | Compound 17ya 9 mg | Placebo |
|---|---|---|
| Serious Adverse Reaction | (N = 149) | (N = 89) |
| COVID-19 | 2.7% | 4.5% |
| Pneumonia | 2.7% | 4.5% |
| Sepsis | 2.7% | 2.2% |
| Septic shock | 2.0% | 7.9% |
| Acute Kidney Injury | 4.0% | 7.9% |
| Acute Respiratory Failure | 3.4% | 4.5% |
| Pulmonary embolism | 2.0% | 3.4% |
| Respiratory Failure | 8.7% | 18.0% |

Overall, the display of serious TEAEs in this combined population is similar to that observed in the Phase 3 Study B, which is expected as the Phase 3 study was much larger than the Phase 2 study.

Laboratory Evaluations, Vital Signs, and Other Safety Evaluations

Study A

There were no clinically meaningful findings in the clinical laboratory assessments (chemistry, hematology, and urinalysis), vital signs, physical examination, and chest X-ray findings.

Study B

In Study B there have been no laboratory abnormalities that were severe TEAEs, SAEs, or led to death. There were no clinically meaningful findings in laboratory assessments, vital signs, physical examinations, or other safety evaluations that could be directly related to the study drug.

The following TEAEs occurred in ≥2% in the Compound 17ya treated group and a higher incidence than observed in the placebo group: alanine aminotransferase increase (3.1% in Compound 17ya group vs. 2.9% in the placebo group), fibrin D-dimer increased (3.1% in Compound 17ya group vs. 2.9% in placebo group), gamma-glutamyltransferase increased (2.3% Compound 17ya group vs. 1.4% placebo group), serum ferritin increased (2.3% Compound 17ya group vs. 2.9% placebo group), and transaminases increased (4.6% Compound 17ya group vs. 2.9% placebo group). Veru considers these observations to be not clinically meaningful. Two patients in the Compound 17ya group and two patients in the placebo group discontinued treatment due to a TEAE for liver function test abnormality.

Safety Conclusions

The totality of evidence supports that Compound 17ya is safe and generally well-tolerated in hospitalized moderate to severe COVID-19 patients at high risk for ARDS as well as in Prostate Cancer patients.

Specifically, the Following Conclusions are Made from COVID-19 Study A:

Overall, no treatment related treatment-emergent adverse events (TEAEs) or other significant TEAEs were reported during the study. There were no treatment-related serious TEAEs and all serious TEAEs were unrelated to study drug. A similar number of subjects (12 subjects each) had TEAEs who received Compound 17ya and placebo. No events were considered to be related to study drug. Most of the TEAEs were reported by single subjects in both treatment groups. The majority of TEAEs were of Grade 1, reported by a total of 13 (33.3%) subjects. Overall, 7 subjects died during the study (1 subject who received Compound 17ya and 6 subjects who received placebo). Eight subjects had serious TEAEs (3 subjects who received Compound 17ya and 5 subjects who received placebo), which were considered by the investigator to be not related or unlikely related to the study drug. There were no other significant TEAEs during the study. There were no clinically meaningful findings in the clinical laboratory assessments (chemistry, hematology, and urinalysis), vital signs, physical examination, ECGs, or chest X-ray findings.

In COVID-19 Study B, the overall conclusions of the safety of Compound 17ya in hospitalized patients with moderate to severe COVID-19 infection who are at high risk for ARDS are the following. The benefit: risk ratio of Compound 17ya is clinically relevant based on the reduction in deaths (51.6% reduction in Compound 17ya group) and reduction in life threatening TEAEs in the Compound 17ya group compared to placebo such as: septic shock (79.2% reduction), pneumonia (52.3% reduction), pneumothorax (92.1% reduction), respiratory failure (50.7% reduction), and acute kidney injury (26.7% reduction), which demonstrates a meaningful representation of pharmacologic benefit and the efficacy of Compound 17ya in the treatment of moderate to severe COVID-19 infection. Compound 17ya was well tolerated compared to placebo in this study. The proportion of subjects experiencing any TEAE was lower in the Compound 17ya treated group (63.1%) than in the placebo group (78.3%). The most common TEAEs reported in the Compound 17ya treated group were: anemia (5.4% Compound 17ya vs. 4.3% placebo); constipation (6.9% Compound 17ya vs. 8.7% placebo); pneumonia (6.2% Compound 17ya vs. 13.0% placebo); urinary tract infection (6.2% Compound 17ya vs. 1.4% placebo); acute kidney injury (8.5% Compound 17ya vs. 11.6% placebo); acute respiratory failure (5.4% Compound 17ya vs. 4.3% placebo); and respiratory failure (10.0% Compound 17ya vs. 20.3% placebo).

All the most common TEAEs reported in the Compound 17ya treated group occurred at a higher rate in the placebo group except for urinary tract infection and acute respiratory failure. While the incidence of acute respiratory failure is slightly higher than in the Compound 17ya treated group compared to placebo, the incidence of respiratory failure is 103% higher in the placebo group compared to the Compound 17ya group. Therefore, it is concluded that this is an anomaly in the preferred terms in the study. There is no mechanism of action or rationale for why there is an imbalance in urinary tract infection in the Compound 17ya treated group.

The SOCs and TEAEs that are observed at a higher rate in the Compound 17ya group compared to the placebo group, SOCs: Blood and lymphatic system disorders, Gastrointestinal disorders, Skin and subcutaneous tissue disorders; TEAEs: anemia, diarrhea, vomiting, urinary tract infections, various skin disorders such as allergic dermatitis, intertrigo, rash, and decubitus ulcer can be managed with therapy in hospitalized patients being treated with Compound 17ya and are not (or are not immediately) life threatening.

The SOCs in which the incidence of TEAEs are at least 20% lower in the Compound 17ya group compared to the placebo group are: Cardiac Disorders (−59.5%); Infections and infestations (−26.1%); Metabolism and nutrition disorders (−37.9%); Musculoskeletal and connective tissue disorders (−47.2%); Nervous system disorders (−40.8%); Psychiatric disorders (−20.7%); Renal and urinary disorders (−38.8%); Respiratory, thoracic, and mediastinal disorders (−45.3%); Vascular disorders (−43.9%).

The proportion of subjects experiencing a serious TEAE was lower in the Compound 17ya treated group (29.2%) compared to the placebo group (46.4%). The most common serious TEAEs in the Compound 17ya treated group were: respiratory failure (10.0% Compound 17ya vs. 20.3% placebo); acute kidney injury (4.6% Compound 17ya vs. 8.7% placebo); and acute respiratory failure (3.8% Compound 17ya vs. 4.3% placebo).

All of these serious TEAEs were experienced by a higher proportion of subjects in the placebo group than in the Compound 17ya treated group.

Overall, TEAEs leading to discontinuation were equal between the treatment groups (4.6% in the Compound 17ya group vs. 4.3% in the placebo group). The preferred terms for the AEs that led to discontinuation in the Compound 17ya group do not represent TEAEs that are observed at a higher rate in the Compound 17ya group (COVID-19 [n=1], endocarditis staphylococcal [n=1], alanine aminotransferase increased [n=1], liver function test increased [n=1], acute kidney injury [n=1], respiratory failure [n=1]), with the exception of dysphagia (n=1) which was not observed in any patients in the placebo group.

Overall, 48 subjects died during the study who were treated with at least one dose of study drug: 23 (17.7%) in the Compound 17ya group and 25 (36.2%) in the placebo group.

There were no clinically meaningful findings in the clinical laboratory assessments (chemistry, hematology, and urinalysis) or in assessments of vital signs, physical examination, ECGs, or chest X-ray.

In studies of Compound 17ya in advanced prostate cancer in which higher doses of Compound 17ya (up to 81 mg) were investigated, the maximum tolerated dose (MTD) was determined to be 63 mg PIC/32 mg FC. Overall, Compound 17ya at the MTD has been well-tolerated, with the most common TEAEs being reported under gastrointestinal disorders (such as diarrhea, fatigue, and nausea) and investigations (such as liver enzyme increases). It is important to note that although the TEAE ‘diarrhea’ is common in both prostate cancer studies, the available data from these studies indicate that the overall percentage of ‘diarrhea’ decreased by approximately 70% in the Phase 3 Prostate Cancer Study compared to the Phase 1b/2 Prostate Cancer Study. This may be due to the change in formulation of Compound 17ya used in Phase 3 (32 mg FC) compared to the Phase 1b/2 (63 mg PIC) which allowed for a decrease in Compound 17ya dose of about 50%. It is important to note that in the Phase 2 and Phase 3 COVID-19 Compound 17ya studies ‘diarrhea’ was not a clinically significant reported safety finding for the 9 mg Compound 17ya dose.

### Example 4

### In Vivo ARDS Mouse Model

In an NIH ARDS mouse model, BALB/c mice infected with mouse-adapted SARS-CoV-2, administered Compound 17ya via oral gavage once daily (3 mg/kg and 9 mg/kg) for 5 days. Comprehensive pathology reports observed that there was a dose dependent reduction in bronchointerstitial inflammation (2 days) and in global pneumonia severity score both at 2 days and 5 days in Compound 17ya treated mice compared to virus vehicle group (see Table 45). If clinical benefit is defined based on the reduction of treatment failures, where treatment failure is death or moderate or marked pneumonia, then Compound 17ya treatment resulted in an 40% absolute reduction of treatment failures as early as Day 2 (4 of 5 mice [80%] in the virus vehicle group versus 4 of 10 mice [40%] in the combined Compound 17ya treated groups) By Day 5, Compound 17ya treatment had a 30% absolute reduction in treatment failures (5 of 5 mice [100%] in the virus vehicle group versus 7 of 10 mice [70%] in the combined Compound 17ya treated groups). Compound 17ya treatment also reduced mortality in mice infected with SARS-CoV-2 with a 40% survival rate in 3 mg/kg and 9 mg/kg Compound 17ya treated groups combined, versus a 20% survival rate in the virus vehicle infected group (see Table 46).

TABLE 45
Observational Assessments of Histopathologic Analysis
of Lungs from Mice with ARDS

| | |
|---|---|
| Treatment | # Animals Affected (%) |

Bronchointerstitial Inflammation (2 days)-
Animals with moderate inflammation

| | | |
|---|---|---|
| Vehicle | 3/5 | (60) |
| 3 mg/kg Compound 17ya | 2/5 | (40) |
| 9 mg/kg Compound 17ya | 1/5 | (20)ª |

Global Pneumonia Severity Score (2 days)-
Animals with moderate or marked pneumonia

| | | |
|---|---|---|
| Vehicle | 4/5 | (80) |
| 3 mg/kg Compound 17ya | 2/5 | (40) |
| 9 mg/kg Compound 17ya | 2/5 | (40)^{b} |

Global Pneumonia Severity Score (5 days)-
Animals with moderate or marked pneumonia

| | | |
|---|---|---|
| Vehicle | 1/1 | (100) |
| 3 mg/kg Compound 17ya | 1/2 | (50) |
| 9 mg/kg Compound 17ya | 0/2 | (0)^{c} |

^{a}67% reduction
^{b}50% reduction
^{c}100% reduction

TABLE 46
Number of Clinical Treatment Failures Defined as Death or Respiratory
Distress (moderate or marked pneumonia) For Each Study Group

| | Treatment Failures | |
|---|---|---|
| Group | Day 2 | Day 5 |
| Vehicle | 4/5 (80%) | 5/5 (100%) |
| 3 mg/kg | 2/5 (40%) | 4/5 (80%) |
| 9 mg/kg | 2/5 (40%) | 3/5 (60%) |
| All treated | 4/10 (40%) | 7/10 (70%) |

Compound 17ya administration in aged BALB/c mice infected with mouse-adapted SARS-CoV-2 when the drug was administered QD starting at the time of infection resulted in a protective effect in survival (40% survival in treated groups vs. 20% survival in the vehicle infected group). As a correlate, daily Compound 17ya administration did result in decreases in markers of lung pathology including broncointerstitial inflammation and the global pneumonia score at both 2 and 5 days. Compound 17ya treatment did not exhibit significant protective effects against weight loss, lung congestion or viral titer in this mouse model. Overall, in this aggressive model of SARS-CoV-2 infection, Compound 17ya had an impact on mortality and lung pathology.

All of the features described herein (including any accompanying claims, abstract and drawings), and/or all of the steps of any method or process so disclosed, may be combined with any of the above aspects in any combination, except combinations where at least some of such features and/or steps are mutually exclusive. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a coronavirus infection in a subject in need thereof by administering to the subject a formulation having a therapeutically effective amount of a compound of Formula (I): 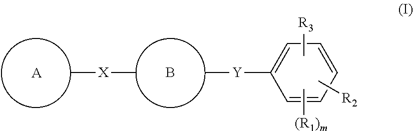
wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole, or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

2. A method of treating a coronavirus infection in a subject who has or is at high risk to develop acute respiratory distress syndrome (ARDS), or high risk of death by administering to the subject a formulation having a therapeutically effective amount of a compound of Formula (I): 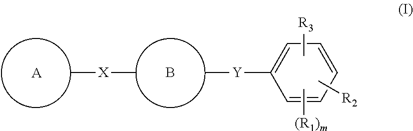
wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole, or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

3. The method according to claim 1 or 2, wherein A is phenyl or indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

4. The method according to claim 1 or 2, wherein A is phenyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

5. The method according to claim 1 or 2, wherein A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO-alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

6. The method according to claim 1 or 2, wherein A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond;
Y is —C═O; and
m is 1-3, or a pharmaceutically acceptable salt, hydrate, polymorph, or isomer thereof.

7. The method according to claim 1, wherein the method reduces viral load,

8. The method according to claim 1, wherein said SARS-CoV-2 infection is any variant of SARS-CoV-2 such as omicron including BA.1 and BA.2 or delta variants, or any descendent variants thereof.

9. The method according to claim 1, wherein said treating reduces morbidity.

10. The method according to claim 1, wherein said morbidity is any one of atrial fibrillation, bradycardia, pneumonia, bacterial pneumonia, hyperkalemia, hypokalemia, hypophosphatemia, chronic bronchitis, hypoxia, pneumothorax, respiratory failure, acute renal injury, cardiac arrest, septic shock, hypotension, or any combination thereof, as compared to a patient population treated with placebo.

11. The method according to claim 1 or 2 of treating a coronavirus infection in a subject in need thereof by administering to the subject a formulation having a therapeutically effective amount of a compound of the Formula VII: 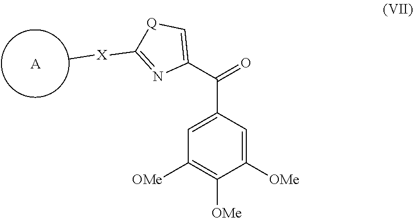
wherein
X is a bond or NH;
Q is NH or S; and
A is a phenyl, indolyl, or indazolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

12. The method according to claim 11, wherein X is a bond.

13. The method according to claim 11, wherein X is NH.

14. The method according to claim 11, wherein X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

15. The method according to of claim 1 or 2 of treating a coronavirus infection in a subject in need thereof by administering to the subject a formulation having a therapeutically effective amount of a compound of the Formula VII(c): 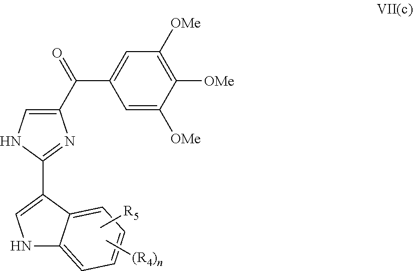
wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

16. The method according to claim 1 or 2 of treating a coronavirus infection by administering to the subject a formulation having a therapeutically effective amount of a compound 17ya represented by:

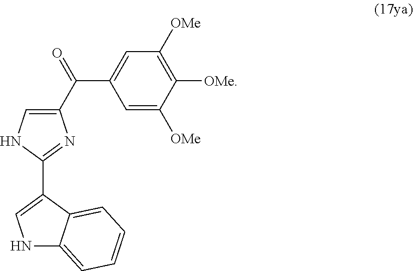 [SMILES: COc1cc(C(=O)c2c[nH]c(-c3c[nH]c4ccccc34)n2)cc(OC)c1OC]

17. The method according to claim 1 or 2, wherein the coronavirus infection is caused by SARS-CoV, MERS-CoV, or SARS-CoV-2.

18. The method according to claim 1 or 2, wherein the coronavirus infection is caused by SARS-CoV-2.

19. The method according to claim 18, wherein said SARS-CoV-2 infection is any variant of SARS-CoV-2 such as omicron including BA.1 and BA.2 or delta variants, or any descendent variant thereof.

20. The method according to claim 18, wherein the method reduces mortality as compared to a patient population treated with placebo.

21. The method according to claim 18, wherein the method reduces morbidity as compared to a patient population treated with placebo.

22. The method according to claim 21, wherein said morbidity is any one of atrial fibrillation, bradycardia, pneumonia, bacterial pneumonia, hyperkalemia, hypokalemia, hypophosphatemia, chronic bronchitis, hypoxia, pneumothorax, respiratory failure, acute renal injury, cardiac arrest, septic shock, hypotension, or any combination thereof, as compared to a patient population treated with placebo.

23. The method according to claim 21, wherein said morbidity is any one of respiratory failure, acute renal injury, cardiac arrest, septic shock, or hypotension, or any combination thereof, as compared to a patient population treated with placebo.

24. The method according to claim 21, wherein the method reduces viral load, respiratory failure, days in ICU, days in the hospital, days on mechanical ventilator, or improves WHO Ordinal Scale for Clinical Improvements as compared to a patient population treated with placebo.

25. The method according to claim 18, wherein the method reduces mortality or respiratory failure in subjects >60 years of age as compared to a patient population treated with placebo.

26. The method according to claim 18, wherein the method reduces mortality or respiratory failure when dosed in combination with remdesivir and/or dexamethasone as compared to a patient population treated with placebo.

27. The method according to any one of claim 18, further comprising a second therapy.

28. The method according to claim 27, wherein the second therapy is remdesivir, dexamethasone or another corticosteroid, or remdesivir plus a corticosteroid.

29. The method according to claim 27, wherein the second therapy is a medication that modulates the immune system or host cell factors, such as dexamethasone or another corticosteroid, an IL-6 inhibitor such as tocilizumab, interferons, an IL-1 inhibitor, or a kinase inhibitor such as baricitinib.

30. The method according to claim 27, wherein the second therapy is an antibody therapy such as high titer COVID-19 convalescent plasma, IVIG, a monoclonal antibody therapy such as casirivimab plus imdevimab, bamlanivimab, bamlanivimab plus etesevimab, tixagevimab plus cilgavimab, or bebtelovimab.

31. The method according to claim 27, wherein the second therapy is a second antiviral therapy that is at least one of favipiravir, lopinavir, ritonavir, remdesivir, molnupiravir, nirmatrelvir plus ritonavir, janus kinase inhibitors, hydroxychloroquine, azithromycin, a neuraminidase inhibitor, amantadine, rimantadine, a hemagglutinin inhibitor, ribavirin, idoxuridine, trifluridine, vidarabine, acyclovir, ganciclovir, foscarnet, zidovudine, didanosine, peramivir, zalcitabine, stavudine, famciclovir, oseltamivir, zanamivir, or valaciclovir.

32. The method according to claim 27, wherein the second therapy is at least one of vitamins C or D, zinc, famotidine, ivermectin, or angiotensin converting enzyme inhibitor (ACEI) or angiotensin receptor binding (ARB) agent.

33. The method according to claim 18, wherein the compound is administered in an amount of about 1 to about 100 mg.

34. The method according to claim 18, wherein the compound is administered in an amount of about 4 mg to about 90 mg.

35. The method according to claim 18, wherein the compound is administered in an amount of about 4 mg to about 45 mg.

36. The methods according to claim 18, wherein the compound is administered in an amount of about 3 mg, or about 9 mg, or about 18 mg.

36. The method according to claim 1 or 2 further comprising a pharmaceutically acceptable excipient.
